SEQUENCE LISTING (931 sequences)

SEQ ID NO: 1 - protein, 10 aa, Homo sapiens
VVVGADGVGK

SEQ ID NO: 2 - protein, 10 aa, synthetic construct
SVVGADGVGK

SEQ ID NO: 3 - protein, 10 aa, synthetic construct
AVVGADGVGK

SEQ ID NO: 4 - protein, 10 aa, Homo sapiens
KLVVVGAVGV

SEQ ID NO: 5 - protein, 10 aa, synthetic construct
KLVVVGAVGV

SEQ ID NO: 6 - protein, 10 aa, synthetic construct
KLVEVGAVGV

SEQ ID NO: 7 - protein, 10 aa, synthetic construct
KLVDVGAVGV

SEQ ID NO: 8 - protein, 10 aa, synthetic construct
FLVVVGAVGV

SEQ ID NO: 9 - protein, 10 aa, synthetic construct
KLVVVGAVGL

SEQ ID NO: 10 - protein, 10 aa, synthetic construct
FLVVVGAVGL

SEQ ID NO: 11 - protein, 10 aa, synthetic construct
KLVEVGAVGL

SEQ ID NO: 12 - protein, 10 aa, synthetic construct
KLVDVGAVGL

SEQ ID NO: 13 - protein, 9 aa, Homo sapiens
LVVVGAVGV

SEQ ID NO: 14 - protein, 9 aa, synthetic construct
LVVEGAVGV

SEQ ID NO: 15 - protein, 9 aa, synthetic construct
LVVDGAVGV

SEQ ID NO: 16 - protein, 9 aa, synthetic construct
LLVVGAVGV

SEQ ID NO: 17 - protein, 9 aa, synthetic construct
LLVEGAVGV

SEQ ID NO: 18 - protein, 9 aa, synthetic construct
LLVDGAVGV

SEQ ID NO: 19 - protein, 9 aa, synthetic construct
LLVVGAVGL

SEQ ID NO: 20 - protein, 9 aa, synthetic construct
LVVEGAVGL

SEQ ID NO: 21 - protein, 9 aa, synthetic construct
LVVDGAVGL

SEQ ID NO: 22 - protein, 10 aa, Homo sapiens
KLVVVGARGV

SEQ ID NO: 23 - protein, 10 aa, synthetic construct
KLVVVGARGV

SEQ ID NO: 24 - protein, 10 aa, synthetic construct
KLVEVGARGV

SEQ ID NO: 25 - protein, 10 aa, synthetic construct
KLVDVGARGV

SEQ ID NO: 26 - protein, 10 aa, synthetic construct
FLVVVGARGV

SEQ ID NO: 27 - protein, 10 aa, synthetic construct
KLVVVGARGL

SEQ ID NO: 28 - protein, 10 aa, synthetic construct
FLVVVGARGL

SEQ ID NO: 29 - protein, 10 aa, synthetic construct
KLVEVGARGL

SEQ ID NO: 30 - protein, 10 aa, synthetic construct
KLVDVGARGL

SEQ ID NO: 31 - protein, 9 aa, Homo sapiens
LVVVGARGV

SEQ ID NO: 32 - protein, 9 aa, synthetic construct
LVVEGARGV

SEQ ID NO: 33 - protein, 9 aa, synthetic construct
LVVDGARGV

SEQ ID NO: 34 - protein, 9 aa, synthetic construct
LLVVGARGV

SEQ ID NO: 35 - protein, 9 aa, synthetic construct
LLVEGARGV

SEQ ID NO: 36 - protein, 9 aa, synthetic construct
LLVDGARGV

SEQ ID NO: 37 - protein, 9 aa, synthetic construct
LLVVGARGL

SEQ ID NO: 38 - protein, 9 aa, synthetic construct
LVVEGARGL

SEQ ID NO: 39 - protein, 9 aa, synthetic construct
LVVDGARGL

SEQ ID NO: 40 - protein, 10 aa, Homo sapiens
KLVVVGADGV

SEQ ID NO: 41 - protein, 10 aa, synthetic construct
KLVVVGADGV

SEQ ID NO: 42 - protein, 10 aa, synthetic construct
KLVEVGADGV

SEQ ID NO: 43 - protein, 10 aa, synthetic construct
KLVDVGADGV

SEQ ID NO: 44 - protein, 10 aa, synthetic construct
FLVVVGADGV

SEQ ID NO: 45 - protein, 10 aa, synthetic construct
KLVVVGADGL

SEQ ID NO: 46 - protein, 10 aa, synthetic construct
FLVVVGADGL

SEQ ID NO: 47 - protein, 10 aa, synthetic construct
KLVEVGADGL

SEQ ID NO: 48 - protein, 10 aa, synthetic construct
KLVDVGADGL

SEQ ID NO: 49 - protein, 9 aa, Homo sapiens
LVVVGADGV

SEQ ID NO: 50 - protein, 9 aa, synthetic construct
LVVEGADGV

SEQ ID NO: 51 - protein, 9 aa, synthetic construct
LVVDGADGV

SEQ ID NO: 52 - protein, 9 aa, synthetic construct
LLVVGADGV

SEQ ID NO: 53 - protein, 9 aa, synthetic construct
LLVEGADGV

SEQ ID NO: 54 - protein, 9 aa, synthetic construct
LLVDGADGV

SEQ ID NO: 55 - protein, 9 aa, synthetic construct
LLVVGADGL

SEQ ID NO: 56 - protein, 9 aa, synthetic construct
LVVEGADGL

SEQ ID NO: 57 - protein, 9 aa, synthetic construct
LVVDGADGL

SEQ ID NO: 58 - protein, 10 aa, Homo sapiens
KLVVVGAAGV

SEQ ID NO: 59 - protein, 10 aa, synthetic construct
KLVVVGAAGV

SEQ ID NO: 60 - protein, 10 aa, synthetic construct
KLVEVGAAGV

SEQ ID NO: 61 - protein, 10 aa, synthetic construct
KLVDVGAAGV

SEQ ID NO: 62 - protein, 10 aa, synthetic construct
FLVVVGAAGV

SEQ ID NO: 63 - protein, 10 aa, synthetic construct
KLVVVGAAGL

SEQ ID NO: 64 - protein, 10 aa, synthetic construct
FLVVVGAAGL

SEQ ID NO: 65 - protein, 10 aa, synthetic construct
KLVEVGAAGL

SEQ ID NO: 66 - protein, 10 aa, synthetic construct
KLVDVGAAGL

SEQ ID NO: 67 - protein, 9 aa, Homo sapiens
LVVVGAAGV

SEQ ID NO: 68 - protein, 9 aa, synthetic construct
LVVEGAAGV

SEQ ID NO: 69 - protein, 9 aa, synthetic construct
LVVDGAAGV

SEQ ID NO: 70 - protein, 9 aa, synthetic construct
LLVVGAAGV

SEQ ID NO: 71 - protein, 9 aa, synthetic construct
LLVEGAAGV

SEQ ID NO: 72 - protein, 9 aa, synthetic construct
LLVDGAAGV

SEQ ID NO: 73 - protein, 9 aa, synthetic construct
LLVVGAAGL

SEQ ID NO: 74 - protein, 9 aa, synthetic construct
LVVEGAAGL

SEQ ID NO: 75 - protein, 9 aa, synthetic construct
LVVDGAAGL

SEQ ID NO: 76 - protein, 10 aa, Homo sapiens
KLVVVGACGV

SEQ ID NO: 77 - protein, 10 aa, synthetic construct
KLVVVGACGV

SEQ ID NO: 78 - protein, 10 aa, synthetic construct
KLVEVGACGV

SEQ ID NO: 79 - protein, 10 aa, synthetic construct
KLVDVGACGV

SEQ ID NO: 80 - protein, 10 aa, synthetic construct
FLVVVGACGV

SEQ ID NO: 81 - protein, 10 aa, synthetic construct
KLVVVGACGL

SEQ ID NO: 82 - protein, 10 aa, synthetic construct
FLVVVGACGL

SEQ ID NO: 83 - protein, 10 aa, synthetic construct
KLVEVGACGL

SEQ ID NO: 84 - protein, 10 aa, synthetic construct
KLVDVGACGL

SEQ ID NO: 85 - protein, 9 aa, Homo sapiens
LVVVGACGV

SEQ ID NO: 86 - protein, 9 aa, synthetic construct
LVVEGACGV

SEQ ID NO: 87 - protein, 9 aa, synthetic construct
LVVDGACGV

SEQ ID NO: 88 - protein, 9 aa, synthetic construct
LLVVGACGV

SEQ ID NO: 89 - protein, 9 aa, synthetic construct
LLVEGACGV

SEQ ID NO: 90 - protein, 9 aa, synthetic construct
LLVDGACGV

SEQ ID NO: 91 - protein, 9 aa, synthetic construct
LLVVGACGL

SEQ ID NO: 92 - protein, 9 aa, synthetic construct
LVVEGACGL

SEQ ID NO: 93 - protein, 9 aa, synthetic construct
LVVDGACGL

SEQ ID NO: 94 - protein, 10 aa, Homo sapiens
VGADGVGKSA

SEQ ID NO: 95 - protein, 10 aa, synthetic construct
VLADGVGKSA

SEQ ID NO: 96 - protein, 10 aa, synthetic construct
VGADGVGKSL

SEQ ID NO: 97 - protein, 10 aa, synthetic construct
VGADGVGKSV

SEQ ID NO: 98 - protein, 10 aa, synthetic construct
VLADGVGKSL

SEQ ID NO: 99 - protein, 10 aa, synthetic construct
VLADGVGKSV

SEQ ID NO: 100 - protein, 9 aa, Homo sapiens
VGADGVGKS

SEQ ID NO: 101 - protein, 9 aa, synthetic construct
VLADGVGKS

SEQ ID NO: 102 - protein, 9 aa, synthetic construct
VGADGVGKL

SEQ ID NO: 103 - protein, 9 aa, synthetic construct
VGADGVGKV

SEQ ID NO: 104 - protein, 9 aa, synthetic construct
VLADGVGKL

SEQ ID NO: 105 - protein, 9 aa, synthetic construct
VLADGVGKL

SEQ ID NO: 106 - protein, 10 aa, Homo sapiens
VGARGVGKSA

SEQ ID NO: 107 - protein, 10 aa, synthetic construct
VLARGVGKSA

SEQ ID NO: 108 - protein, 10 aa, synthetic construct
VGARGVGKSL

SEQ ID NO: 109 - protein, 10 aa, synthetic construct
VGARGVGKSV

SEQ ID NO: 110 - protein, 10 aa, synthetic construct
VLARGVGKSL

SEQ ID NO: 111 - protein, 10 aa, synthetic construct
VLARGVGKSV

SEQ ID NO: 112 - protein, 9 aa, Homo sapiens
VGARGVGKS

SEQ ID NO: 113 - protein, 9 aa, synthetic construct
VLARGVGKS

SEQ ID NO: 114 - protein, 9 aa, synthetic construct
VGARGVGKL

SEQ ID NO: 115 - protein, 9 aa, synthetic construct
VGARGVGKV

SEQ ID NO: 116 - protein, 9 aa, synthetic construct
VLARGVGKL

SEQ ID NO: 117 - protein, 9 aa, synthetic construct
VLARGVGKL

SEQ ID NO: 118 - protein, 10 aa, Homo sapiens
VGAVGVGKSA

SEQ ID NO: 119 - protein, 10 aa, synthetic construct
VLAVGVGKSA

SEQ ID NO: 120 - protein, 10 aa, synthetic construct
VGAVGVGKSL

SEQ ID NO: 121 - protein, 10 aa, synthetic construct
VGAVGVGKSV

SEQ ID NO: 122 - protein, 10 aa, synthetic construct
VLAVGVGKSL

SEQ ID NO: 123 - protein, 10 aa, synthetic construct
VLAVGVGKSV

SEQ ID NO: 124 - protein, 9 aa, Homo sapiens
VGAVGVGKS

SEQ ID NO: 125 - protein, 9 aa, synthetic construct
VLAVGVGKS

SEQ ID NO: 126 - protein, 9 aa, synthetic construct
VGAVGVGKL

SEQ ID NO: 127 - protein, 9 aa, synthetic construct
VGAVGVGKV

SEQ ID NO: 128 - protein, 9 aa, synthetic construct
VLAVGVGKL

SEQ ID NO: 129 - protein, 9 aa, synthetic construct
VLAVGVGKL

SEQ ID NO: 130 - protein, 10 aa, Homo sapiens
VGAAGVGKSA

SEQ ID NO: 131 - protein, 10 aa, synthetic construct
VLAAGVGKSA

SEQ ID NO: 132 - protein, 10 aa, synthetic construct
VGAAGVGKSL

SEQ ID NO: 133 - protein, 10 aa, synthetic construct
VGAAGVGKSV

SEQ ID NO: 134 - protein, 10 aa, synthetic construct
VLAAGVGKSL

SEQ ID NO: 135 - protein, 10 aa, synthetic construct
VLAAGVGKSV

SEQ ID NO: 136 - protein, 9 aa, Homo sapiens
VGAAGVGKS

SEQ ID NO: 137 - protein, 9 aa, synthetic construct
VLAAGVGKS

SEQ ID NO: 138 - protein, 9 aa, synthetic construct
VGAAGVGKL

SEQ ID NO: 139 - protein, 9 aa, synthetic construct
VGAAGVGKV

SEQ ID NO: 140 - protein, 9 aa, synthetic construct
VLAAGVGKL

SEQ ID NO: 141 - protein, 9 aa, synthetic construct
VLAAGVGKL

SEQ ID NO: 142 - protein, 10 aa, Homo sapiens
VGACGVGKSA

SEQ ID NO: 143 - protein, 10 aa, synthetic construct
VLACGVGKSA

SEQ ID NO: 144 - protein, 10 aa, synthetic construct
VGACGVGKSV

SEQ ID NO: 145 - protein, 10 aa, synthetic construct
VLACGVGKSL

SEQ ID NO: 146 - protein, 10 aa, synthetic construct
VLACGVGKSV

SEQ ID NO: 147 - protein, 9 aa, Homo sapiens
VGACGVGKS

SEQ ID NO: 148 - protein, 9 aa, synthetic construct
VLACGVGKS

SEQ ID NO: 149 - protein, 9 aa, synthetic construct
VGACGVGKL

SEQ ID NO: 150 - protein, 9 aa, synthetic construct
VGACGVGKV

SEQ ID NO: 151 - protein, 9 aa, synthetic construct
VLACGVGKL

SEQ ID NO: 152 - protein, 9 aa, synthetic construct
VLACGVGKL

SEQ ID NO: 153 - protein, 10 aa, Homo sapiens
VVVGADGVGK

SEQ ID NO: 154 - protein, 10 aa, synthetic construct
VLVGADGVGK

SEQ ID NO: 155 - protein, 10 aa, synthetic construct
RVVGADGVGK

SEQ ID NO: 156 - protein, 10 aa, synthetic construct
RLVGADGVGK

SEQ ID NO: 157 - protein, 10 aa, synthetic construct
VVVGADLVGK

SEQ ID NO: 158 - protein, 10 aa, synthetic construct
VLVGADLVGK

SEQ ID NO: 159 - protein, 10 aa, synthetic construct
RVVGADLVGK

SEQ ID NO: 160 - protein, 10 aa, synthetic construct
RLVGADLVGK

SEQ ID NO: 161 - protein, 10 aa, synthetic construct
VLVGADGVGK

SEQ ID NO: 162 - protein, 10 aa, synthetic construct
KLVGADGVGK

SEQ ID NO: 163 - protein, 9 aa, Homo sapiens
VVGARGVGK

SEQ ID NO: 164 - protein, 9 aa, synthetic construct
VLGARGVGK

SEQ ID NO: 165 - protein, 9 aa, synthetic construct
RVGARGVGK

SEQ ID NO: 166 - protein, 9 aa, synthetic construct
VVGARGLGK

SEQ ID NO: 167 - protein, 9 aa, synthetic construct
VLGARGLGK

SEQ ID NO: 168 - protein, 9 aa, synthetic construct
RLGARGLGK

SEQ ID NO: 169 - protein, 10 aa, Homo sapiens
VVVGAVGVGK

SEQ ID NO: 170 - protein, 10 aa, synthetic construct
VLVGAVGVGK

SEQ ID NO: 171 - protein, 10 aa, synthetic construct
RVVGAVGVGK

SEQ ID NO: 172 - protein, 10 aa, synthetic construct
RLVGAVGVGK

SEQ ID NO: 173 - protein, 10 aa, synthetic construct
VVVGAVLVGK

SEQ ID NO: 174 - protein, 10 aa, synthetic construct
VLVGAVLVGK

SEQ ID NO: 175 - protein, 10 aa, synthetic construct
RVVGAVLVGK

SEQ ID NO: 176 - protein, 10 aa, synthetic construct
RLVGAVLVGK

SEQ ID NO: 177 - protein, 10 aa, synthetic construct
VLVGAVGVGK

SEQ ID NO: 178 - protein, 10 aa, synthetic construct
KVVGAVGVGK

SEQ ID NO: 179 - protein, 10 aa, synthetic construct
KLVGAVGVGK

SEQ ID NO: 180 - protein, 9 aa, Homo sapiens
VVGAVGVGK

SEQ ID NO: 181 - protein, 9 aa, synthetic construct
VLGAVGVGK

SEQ ID NO: 182 - protein, 9 aa, synthetic construct
RVGAVGVGK

SEQ ID NO: 183 - protein, 9 aa, synthetic construct
VVGAVGLGK

SEQ ID NO: 184 - protein, 9 aa, synthetic construct
VLGAVGLGK

SEQ ID NO: 185 - protein, 9 aa, synthetic construct
RLGAVGLGK

SEQ ID NO: 186 - protein, 10 aa, Homo sapiens
VVVGACGVGK

SEQ ID NO: 187 - protein, 10 aa, synthetic construct
VLVGACGVGK

SEQ ID NO: 188 - protein, 10 aa, synthetic construct
RVVGACGVGK

SEQ ID NO: 189 - protein, 10 aa, synthetic construct
RLVGACGVGK

SEQ ID NO: 190 - protein, 10 aa, synthetic construct
VVVGACLVGK

SEQ ID NO: 191 - protein, 10 aa, synthetic construct
VLVGACLVGK

SEQ ID NO: 192 - protein, 10 aa, synthetic construct
RVVGACLVGK

SEQ ID NO: 193 - protein, 10 aa, synthetic construct
RLVGACLVGK

SEQ ID NO: 194 - protein, 10 aa, synthetic construct
VLVGACGVGK

SEQ ID NO: 195 - protein, 10 aa, synthetic construct
KVVGACGVGK

SEQ ID NO: 196 - protein, 10 aa, synthetic construct
KLVGACGVGK

SEQ ID NO: 197 - protein, 9 aa, Homo sapiens
VVGACGVGK

SEQ ID NO: 198 - protein, 9 aa, synthetic construct
VLGACGVGK

SEQ ID NO: 199 - protein, 9 aa, synthetic construct
RVGACGVGK

SEQ ID NO: 200 - protein, 9 aa, synthetic construct
VVGACGLGK

SEQ ID NO: 201 - protein, 9 aa, synthetic construct
VLGACGLGK

SEQ ID NO: 202 - protein, 9 aa, synthetic construct
RLGACGLGK

SEQ ID NO: 203 - protein, 10 aa, Homo sapiens
VVVGARGVGK

SEQ ID NO: 204 - protein, 10 aa, synthetic construct
VLVGARGVGK

SEQ ID NO: 205 - protein, 10 aa, synthetic construct
RVVGARGVGK

SEQ ID NO: 206 - protein, 10 aa, synthetic construct
RLVGARGVGK

SEQ ID NO: 207 - protein, 10 aa, synthetic construct
VVVGARLVGK

SEQ ID NO: 208 - protein, 10 aa, synthetic construct
VLVGARLVGK

SEQ ID NO: 209 - protein, 10 aa, synthetic construct
RVVGARLVGK

SEQ ID NO: 210 - protein, 10 aa, synthetic construct
RLVGARLVGK

SEQ ID NO: 211 - protein, 10 aa, synthetic construct
VLVGARGVGK

SEQ ID NO: 212 - protein, 10 aa, synthetic construct
KVVGARGVGK

SEQ ID NO: 213 - protein, 10 aa, synthetic construct
KLVGARGVGK

SEQ ID NO: 214 - protein, 9 aa, Homo sapiens
VVGADGVGK

SEQ ID NO: 215 - protein, 9 aa, synthetic construct
VLGADGVGK

SEQ ID NO: 216 - protein, 9 aa, synthetic construct
RVGADGVGK

SEQ ID NO: 217 - protein, 9 aa, synthetic construct
VVGADGLGK

SEQ ID NO: 218 - protein, 9 aa, synthetic construct
VLGADGLGK

SEQ ID NO: 219 - protein, 9 aa, synthetic construct
RLGADGLGK

SEQ ID NO: 220 - protein, 10 aa, Homo sapiens
VVVGAAGVGK

SEQ ID NO: 221 - protein, 10 aa, synthetic construct
VLVGAAGVGK

SEQ ID NO: 222 - protein, 10 aa, synthetic construct
RVVGAAGVGK

SEQ ID NO: 223 - protein, 10 aa, synthetic construct
RLVGAAGVGK

SEQ ID NO: 224 - protein, 10 aa, synthetic construct
VVVGAALVGK

SEQ ID NO: 225 - protein, 10 aa, synthetic construct
VLVGAALVGK

SEQ ID NO: 226 - protein, 10 aa, synthetic construct
RVVGAALVGK

SEQ ID NO: 227 - protein, 10 aa, synthetic construct
RLVGAALVGK

SEQ ID NO: 228 - protein, 10 aa, synthetic construct
VLVGAAGVGK

SEQ ID NO: 229 - protein, 10 aa, synthetic construct
KVVGAAGVGK

SEQ ID NO: 230 - protein, 10 aa, synthetic construct
KLVGAAGVGK

SEQ ID NO: 231 - protein, 9 aa, Homo sapiens
VVGAAGVGK

SEQ ID NO: 232 - protein, 9 aa, synthetic construct
VLGAAGVGK

SEQ ID NO: 233 - protein, 9 aa, synthetic construct
RVGAAGVGK

SEQ ID NO: 234 - protein, 9 aa, synthetic construct
VVGAAGLGK

SEQ ID NO: 235 - protein, 9 aa, synthetic construct
VLGAAGLGK

SEQ ID NO: 236 - protein, 9 aa, synthetic construct
RLGAAGLGK

SEQ ID NO: 237 - protein, 10 aa, Homo sapiens
VVVGADGVGK

SEQ ID NO: 238 - protein, 10 aa, synthetic construct
SVVGADGVGK

SEQ ID NO: 239 - protein, 10 aa, synthetic construct
VTVGADGVGK

SEQ ID NO: 240 - protein, 10 aa, synthetic construct
VSVGADGVGK

SEQ ID NO: 241 - protein, 10 aa, synthetic construct
AVVGADGVGK

SEQ ID NO: 242 - protein, 10 aa, synthetic construct
VVVGADGLGK

SEQ ID NO: 243 - protein, 10 aa, synthetic construct
STVGADGVGK

SEQ ID NO: 244 - protein, 10 aa, synthetic construct
ATVGADGVGK

SEQ ID NO: 245 - protein, 9 aa, Homo sapiens
VVGADGVGK

SEQ ID NO: 246 - protein, 9 aa, synthetic construct
SVGADGVGK

SEQ ID NO: 247 - protein, 9 aa, synthetic construct
VTGADGVGK

SEQ ID NO: 248 - protein, 9 aa, synthetic construct
AVGADGVGK

SEQ ID NO: 249 - protein, 9 aa, synthetic construct
VVGADGVGK

SEQ ID NO: 250 - protein, 9 aa, synthetic construct
STGADGVGK

SEQ ID NO: 251 - protein, 9 aa, synthetic construct
ATGADGVGK

SEQ ID NO: 252 - protein, 9 aa, synthetic construct
VVGADGLGK

SEQ ID NO: 253 - protein, 10 aa, Homo sapiens
VVVGARGVGK

SEQ ID NO: 254 - protein, 10 aa, synthetic construct
SVVGARGVGK

SEQ ID NO: 255 - protein, 10 aa, synthetic construct
VTVGARGVGK

SEQ ID NO: 256 - protein, 10 aa, synthetic construct
VSVGARGVGK

SEQ ID NO: 257 - protein, 10 aa, synthetic construct
AVVGARGVGK

SEQ ID NO: 258 - protein, 10 aa, synthetic construct
VVVGARGLGK

SEQ ID NO: 259 - protein, 10 aa, synthetic construct
STVGARGVGK

SEQ ID NO: 260 - protein, 10 aa, synthetic construct
ATVGARGVGK

SEQ ID NO: 261 - protein, 9 aa, Homo sapiens
VVGARGVGK

SEQ ID NO: 262 - protein, 9 aa, synthetic construct
SVGARGVGK

SEQ ID NO: 263 - protein, 9 aa, synthetic construct
VTGARGVGK

SEQ ID NO: 264 - protein, 9 aa, synthetic construct
AVGARGVGK

SEQ ID NO: 265 - protein, 9 aa, synthetic construct
VVGARGVGK

SEQ ID NO: 266 - protein, 9 aa, synthetic construct
STGARGVGK

SEQ ID NO: 267 - protein, 9 aa, synthetic construct
ATGARGVGK

SEQ ID NO: 268 - protein, 9 aa, synthetic construct
VVGARGLGK

SEQ ID NO: 269 - protein, 10 aa, Homo sapiens
VVVGAVGVGK

SEQ ID NO: 270 - protein, 10 aa, synthetic construct
SVVGAVGVGK

SEQ ID NO: 271 - protein, 10 aa, synthetic construct
VTVGAVGVGK

SEQ ID NO: 272 - protein, 10 aa, synthetic construct
VSVGAVGVGK

SEQ ID NO: 273 - protein, 10 aa, synthetic construct
AVVGAVGVGK

SEQ ID NO: 274 - protein, 10 aa, synthetic construct
VVVGAVGLGK

SEQ ID NO: 275 - protein, 10 aa, synthetic construct
STVGAVGVGK

SEQ ID NO: 276 - protein, 10 aa, synthetic construct
ATVGAVGVGK

SEQ ID NO: 277 - protein, 9 aa, Homo sapiens
VVGAVGVGK

SEQ ID NO: 278 - protein, 9 aa, synthetic construct
SVGAVGVGK

SEQ ID NO: 279 - protein, 9 aa, synthetic construct
VTGAVGVGK

SEQ ID NO: 280 - protein, 9 aa, synthetic construct
AVGAVGVGK

SEQ ID NO: 281 - protein, 9 aa, synthetic construct
VVGAVGVGK

SEQ ID NO: 282 - protein, 9 aa, synthetic construct
STGAVGVGK

SEQ ID NO: 283 - protein, 9 aa, synthetic construct
ATGAVGVGK

SEQ ID NO: 284 - protein, 9 aa, synthetic construct
VVGAVGLGK

SEQ ID NO: 285 - protein, 10 aa, Homo sapiens
VVVGACGVGK

SEQ ID NO: 286 - protein, 10 aa, synthetic construct
SVVGACGVGK

SEQ ID NO: 287 - protein, 10 aa, synthetic construct
VTVGACGVGK

SEQ ID NO: 288 - protein, 10 aa, synthetic construct
VSVGACGVGK

SEQ ID NO: 289 - protein, 10 aa, synthetic construct
AVVGACGVGK

SEQ ID NO: 290 - protein, 10 aa, synthetic construct
VVVGACGLGK

SEQ ID NO: 291 - protein, 10 aa, synthetic construct
STVGACGVGK

SEQ ID NO: 292 - protein, 10 aa, synthetic construct
ATVGACGVGK

SEQ ID NO: 293 - protein, 9 aa, Homo sapiens
VVGACGVGK

SEQ ID NO: 294 - protein, 9 aa, synthetic construct
SVGACGVGK

SEQ ID NO: 295 - protein, 9 aa, synthetic construct
VTGACGVGK

SEQ ID NO: 296 - protein, 9 aa, synthetic construct
AVGACGVGK

SEQ ID NO: 297 - protein, 9 aa, synthetic construct
VVGACGVGK

SEQ ID NO: 298 - protein, 9 aa, synthetic construct
STGACGVGK

SEQ ID NO: 299 - protein, 9 aa, synthetic construct
ATGACGVGK

SEQ ID NO: 300 - protein, 9 aa, synthetic construct
VVGACGLGK

SEQ ID NO: 301 - protein, 10 aa, Homo sapiens
VVVGAAGVGK

SEQ ID NO: 302 - protein, 10 aa, synthetic construct
SVVGAAGVGK

SEQ ID NO: 303 - protein, 10 aa, synthetic construct
VTVGAAGVGK

SEQ ID NO: 304 - protein, 10 aa, synthetic construct
VSVGAAGVGK

SEQ ID NO: 305 - protein, 10 aa, synthetic construct
AVVGAAGVGK

SEQ ID NO: 306 - protein, 10 aa, synthetic construct
VVVGAAGLGK

SEQ ID NO: 307 - protein, 10 aa, synthetic construct
STVGAAGVGK

SEQ ID NO: 308 - protein, 10 aa, synthetic construct
ATVGAAGVGK

SEQ ID NO: 309 - protein, 9 aa, Homo sapiens
VVGAAGVGK

SEQ ID NO: 310 - protein, 9 aa, synthetic construct
SVGAAGVGK

SEQ ID NO: 311 - protein, 9 aa, synthetic construct
VTGAAGVGK

SEQ ID NO: 312 - protein, 9 aa, synthetic construct
AVGAAGVGK

SEQ ID NO: 313 - protein, 9 aa, synthetic construct
VVGAAGVGK

SEQ ID NO: 314 - protein, 9 aa, synthetic construct
STGAAGVGK

SEQ ID NO: 315 - protein, 9 aa, synthetic construct
ATGAAGVGK

SEQ ID NO: 316 - protein, 9 aa, synthetic construct
VVGAAGLGK

SEQ ID NO: 317 - protein, 10 aa, Homo sapiens
VVVGADGVGK

SEQ ID NO: 318 - protein, 10 aa, synthetic construct
VYVGADGVGK

SEQ ID NO: 319 - protein, 10 aa, synthetic construct
VVVGADGVGF

SEQ ID NO: 320 - protein, 10 aa, synthetic construct
VVLGADGVGK

SEQ ID NO: 321 - protein, 10 aa, synthetic construct
VFVGADGVGK

SEQ ID NO: 322 - protein, 10 aa, synthetic construct
VVVGADGVGL

SEQ ID NO: 323 - protein, 10 aa, synthetic construct
VYVGADGVGF

SEQ ID NO: 324 - protein, 10 aa, synthetic construct
VFVGADGVGL

SEQ ID NO: 325 - protein, 9 aa, Homo sapiens
VVGADGVGK

SEQ ID NO: 326 - protein, 9 aa, synthetic construct
VYGADGVGK

SEQ ID NO: 327 - protein, 9 aa, synthetic construct
VVGADGVGF

SEQ ID NO: 328 - protein, 9 aa, synthetic construct
VVLADGVGK

SEQ ID NO: 329 - protein, 9 aa, synthetic construct
VFGADGVGK

SEQ ID NO: 330 - protein, 9 aa, synthetic construct
VVGADGVGL

SEQ ID NO: 331 - protein, 9 aa, synthetic construct
VYGADGVGF

SEQ ID NO: 332 - protein, 9 aa, synthetic construct
VFGADGVGL

SEQ ID NO: 333 - protein, 10 aa, Homo sapiens
VVVGARGVGK

SEQ ID NO: 334 - protein, 10 aa, synthetic construct
VYVGARGVGK

SEQ ID NO: 335 - protein, 10 aa, synthetic construct
VVVGARGVGF

SEQ ID NO: 336 - protein, 10 aa, synthetic construct
VVLGARGVGK

SEQ ID NO: 337 - protein, 10 aa, synthetic construct
VFVGARGVGK

SEQ ID NO: 338 - protein, 10 aa, synthetic construct
VVVGARGVGL

SEQ ID NO: 339 - protein, 10 aa, synthetic construct
VYVGARGVGF

SEQ ID NO: 340 - protein, 10 aa, synthetic construct
VFVGARGVGL

SEQ ID NO: 341 - protein, 9 aa, Homo sapiens
VVGARGVGK

SEQ ID NO: 342 - protein, 9 aa, synthetic construct
VYGARGVGK

SEQ ID NO: 343 - protein, 9 aa, synthetic construct
VVGARGVGF

SEQ ID NO: 344 - protein, 9 aa, synthetic construct
VVLARGVGK

SEQ ID NO: 345 - protein, 9 aa, synthetic construct
VFGARGVGK

SEQ ID NO: 346 - protein, 9 aa, synthetic construct
VVGARGVGL

SEQ ID NO: 347 - protein, 9 aa, synthetic construct
VYGARGVGF

SEQ ID NO: 348 - protein, 9 aa, synthetic construct
VFGARGVGL

SEQ ID NO: 349 - protein, 10 aa, Homo sapiens
VVVGAVGVGK

SEQ ID NO: 350 - protein, 10 aa, synthetic construct
VYVGAVGVGK

SEQ ID NO: 351 - protein, 10 aa, synthetic construct
VVVGAVGVGF

SEQ ID NO: 352 - protein, 10 aa, synthetic construct
VVLGAVGVGK

SEQ ID NO: 353 - protein, 10 aa, synthetic construct
VFVGAVGVGK

SEQ ID NO: 354 - protein, 10 aa, synthetic construct
VVVGAVGVGL

SEQ ID NO: 355 - protein, 10 aa, synthetic construct
VYVGAVGVGF

SEQ ID NO: 356 - protein, 10 aa, synthetic construct
VFVGAVGVGL

SEQ ID NO: 357 - protein, 9 aa, Homo sapiens
VVGAVGVGK

SEQ ID NO: 358 - protein, 9 aa, synthetic construct
VYGAVGVGK

SEQ ID NO: 359 - protein, 9 aa, synthetic construct
VVGAVGVGF

SEQ ID NO: 360 - protein, 9 aa, synthetic construct
VVLAVGVGK

SEQ ID NO: 361 - protein, 9 aa, synthetic construct
VFGAVGVGK

SEQ ID NO: 362 - protein, 9 aa, synthetic construct
VVGAVGVGL

SEQ ID NO: 363 - protein, 9 aa, synthetic construct
VYGAVGVGF

SEQ ID NO: 364 - protein, 9 aa, synthetic construct
VFGAVGVGL

SEQ ID NO: 365 - protein, 10 aa, Homo sapiens
VVVGACGVGK

SEQ ID NO: 366 - protein, 10 aa, synthetic construct
VYVGACGVGK

SEQ ID NO: 367 - protein, 10 aa, synthetic construct
VVVGACGVGF

SEQ ID NO: 368 - protein, 10 aa, synthetic construct
VVLGACGVGK

SEQ ID NO: 369 - protein, 10 aa, synthetic construct
VFVGACGVGK

SEQ ID NO: 370 - protein, 10 aa, synthetic construct
VVVGACGVGL

SEQ ID NO: 371 - protein, 10 aa, synthetic construct
VYVGACGVGF

SEQ ID NO: 372 - protein, 10 aa, synthetic construct
VFVGACGVGL

SEQ ID NO: 373 - protein, 9 aa, Homo sapiens
VVGACGVGK

SEQ ID NO: 374 - protein, 9 aa, synthetic construct
VYGACGVGK

SEQ ID NO: 375 - protein, 9 aa, synthetic construct
VVGACGVGF

SEQ ID NO: 376 - protein, 9 aa, synthetic construct
VVLACGVGK

SEQ ID NO: 377 - protein, 9 aa, synthetic construct
VFGACGVGK

SEQ ID NO: 378 - protein, 9 aa, synthetic construct
VVGACGVGL

SEQ ID NO: 379 - protein, 9 aa, synthetic construct
VYGACGVGF

SEQ ID NO: 380 - protein, 9 aa, synthetic construct
VFGACGVGL

SEQ ID NO: 381 - protein, 10 aa, Homo sapiens
VVVGAAGVGK

SEQ ID NO: 382 - protein, 10 aa, synthetic construct
VYVGAAGVGK

SEQ ID NO: 383 - protein, 10 aa, synthetic construct
VVVGAAGVGF

SEQ ID NO: 384 - protein, 10 aa, synthetic construct
VVLGAAGVGK

SEQ ID NO: 385 - protein, 10 aa, synthetic construct
VFVGAAGVGK

SEQ ID NO: 386 - protein, 10 aa, synthetic construct
VVVGAAGVGL

SEQ ID NO: 387 - protein, 10 aa, synthetic construct
VYVGAAGVGF

SEQ ID NO: 388 - protein, 10 aa, synthetic construct
VFVGAAGVGL

SEQ ID NO: 389 - protein, 9 aa, Homo sapiens
VVGAAGVGK

SEQ ID NO: 390 - protein, 9 aa, synthetic construct
VYGAAGVGK

SEQ ID NO: 391 - protein, 9 aa, synthetic construct
VVGAAGVGF

SEQ ID NO: 392 - protein, 9 aa, synthetic construct
VVLAAGVGK

SEQ ID NO: 393 - protein, 9 aa, synthetic construct
VFGAAGVGK

SEQ ID NO: 394 - protein, 9 aa, synthetic construct
VVGAAGVGL

SEQ ID NO: 395 - protein, 9 aa, synthetic construct
VYGAAGVGF

SEQ ID NO: 396 - protein, 9 aa, synthetic construct
VFGAAGVGL

SEQ ID NO: 397 - protein, 10 aa, Homo sapiens
VGADGVGKSA

SEQ ID NO: 398 - protein, 10 aa, synthetic construct
VYADGVGKSA

SEQ ID NO: 399 - protein, 10 aa, synthetic construct
VGADGVGKSF

SEQ ID NO: 400 - protein, 10 aa, synthetic construct
VYADGVGKSF

SEQ ID NO: 401 - protein, 10 aa, synthetic construct
VFADGVGKSA

SEQ ID NO: 402 - protein, 10 aa, synthetic construct
VGADGVGKSL

SEQ ID NO: 403 - protein, 10 aa, synthetic construct
VFADGVGKSL

SEQ ID NO: 404 - protein, 10 aa, synthetic construct
VYADGVGKSL

SEQ ID NO: 405 - protein, 10 aa, Homo sapiens
VGAVGVGKSA

SEQ ID NO: 406 - protein, 10 aa, synthetic construct
VYAVGVGKSA

SEQ ID NO: 407 - protein, 10 aa, synthetic construct
VGAVGVGKSF

SEQ ID NO: 408 - protein, 10 aa, synthetic construct
VYAVGVGKSF

SEQ ID NO: 409 - protein, 10 aa, synthetic construct
VFAVGVGKSA

SEQ ID NO: 410 - protein, 10 aa, synthetic construct
VGAVGVGKSL

SEQ ID NO: 411 - protein, 10 aa, synthetic construct
VFAVGVGKSL

SEQ ID NO: 412 - protein, 10 aa, synthetic construct
VYAVGVGKSL

SEQ ID NO: 413 - protein, 10 aa, Homo sapiens
VGARGVGKSA

SEQ ID NO: 414 - protein, 10 aa, synthetic construct
VYARGVGKSA

SEQ ID NO: 415 - protein, 10 aa, synthetic construct
VGARGVGKSF

SEQ ID NO: 416 - protein, 10 aa, synthetic construct
VYARGVGKSF

SEQ ID NO: 417 - protein, 10 aa, synthetic construct
VFARGVGKSA

SEQ ID NO: 418 - protein, 10 aa, synthetic construct
VGARGVGKSL

SEQ ID NO: 419 - protein, 10 aa, synthetic construct
VFARGVGKSL

SEQ ID NO: 420 - protein, 10 aa, synthetic construct
VYARGVGKSL

SEQ ID NO: 421 - protein, 10 aa, Homo sapiens
VGAAGVGKSA

SEQ ID NO: 422 - protein, 10 aa, synthetic construct
VYAAGVGKSA

SEQ ID NO: 423 - protein, 10 aa, synthetic construct
VGAAGVGKSF

SEQ ID NO: 424 - protein, 10 aa, synthetic construct
VYAAGVGKSF

SEQ ID NO: 425 - protein, 10 aa, synthetic construct
VFAAGVGKSA

SEQ ID NO: 426 - protein, 10 aa, synthetic construct
VGAAGVGKSL

SEQ ID NO: 427 - protein, 10 aa, synthetic construct
VFAAGVGKSL

SEQ ID NO: 428 - protein, 10 aa, synthetic construct
VYAAGVGKSL

SEQ ID NO: 429 - protein, 10 aa, Homo sapiens
VGACGVGKSA

SEQ ID NO: 430 - protein, 10 aa, synthetic construct
VYACGVGKSA

SEQ ID NO: 431 - protein, 10 aa, synthetic construct
VGACGVGKSF

SEQ ID NO: 432 - protein, 10 aa, synthetic construct
VYACGVGKSF

SEQ ID NO: 433 - protein, 10 aa, synthetic construct
VFACGVGKSA

SEQ ID NO: 434 - protein, 10 aa, synthetic construct
VGACGVGKSL

SEQ ID NO: 435 - protein, 10 aa, synthetic construct
VFACGVGKSL

SEQ ID NO: 436 - protein, 10 aa, synthetic construct
VYACGVGKSL

SEQ ID NO: 437 - protein, 10 aa, Homo sapiens
VVVGARGVGK

SEQ ID NO: 438 - protein, 10 aa, synthetic construct
RVVGARGVGK

SEQ ID NO: 439 - protein, 10 aa, synthetic construct
VVKGARGVGK

SEQ ID NO: 440 - protein, 10 aa, synthetic construct
VTVGARGVGK

SEQ ID NO: 441 - protein, 10 aa, synthetic construct
VVVGARGPGK

SEQ ID NO: 442 - protein, 10 aa, synthetic construct
VVVGARRVGK

SEQ ID NO: 443 - protein, 10 aa, synthetic construct
KVVGARGVGK

SEQ ID NO: 444 - protein, 9 aa, Homo sapiens
VVGARGVGK

SEQ ID NO: 445 - protein, 9 aa, synthetic construct
VVKARGVGK

SEQ ID NO: 446 - protein, 9 aa, synthetic construct
KVGARGVGK

SEQ ID NO: 447 - protein, 9 aa, synthetic construct
VTGARGVGK

SEQ ID NO: 448 - protein, 9 aa, synthetic construct
VVGARGPGK

SEQ ID NO: 449 - protein, 9 aa, synthetic construct
KVKARGVGK

SEQ ID NO: 450 - protein, 9 aa, synthetic construct
KTGARGVGK

SEQ ID NO: 451 - protein, 10 aa, Homo sapiens
VVVGAVGVGK

SEQ ID NO: 452 - protein, 10 aa, synthetic construct
RVVGAVGVGK

SEQ ID NO: 453 - protein, 10 aa, synthetic construct
VVKGAVGVGK

SEQ ID NO: 454 - protein, 10 aa, synthetic construct
VTVGAVGVGK

SEQ ID NO: 455 - protein, 10 aa, synthetic construct
VVVGAVGPGK

SEQ ID NO: 456 - protein, 10 aa, synthetic construct
VVVGAVRVGK

SEQ ID NO: 457 - protein, 10 aa, synthetic construct
KVVGAVGVGK

SEQ ID NO: 458 - protein, 9 aa, Homo sapiens
VVGAVGVGK

SEQ ID NO: 459 - protein, 9 aa, synthetic construct
VVKAVGVGK

SEQ ID NO: 460 - protein, 9 aa, synthetic construct
KVGAVGVGK

SEQ ID NO: 461 - protein, 9 aa, synthetic construct
VTGAVGVGK

SEQ ID NO: 462 - protein, 9 aa, synthetic construct
VVGAVGPGK

SEQ ID NO: 463 - protein, 9 aa, synthetic construct
KVKAVGVGK

SEQ ID NO: 464 - protein, 9 aa, synthetic construct
KTGAVGVGK

SEQ ID NO: 465 - protein, 10 aa, Homo sapiens
VVVGADGVGK

SEQ ID NO: 466 - protein, 10 aa, synthetic construct
RVVGADGVGK

SEQ ID NO: 467 - protein, 10 aa, synthetic construct
VVKGADGVGK

SEQ ID NO: 468 - protein, 10 aa, synthetic construct
VTVGADGVGK

SEQ ID NO: 469 - protein, 10 aa, synthetic construct
VVVGADGPGK

SEQ ID NO: 470 - protein, 10 aa, synthetic construct
VVVGADRVGK

SEQ ID NO: 471 - protein, 10 aa, synthetic construct
KVVGADGVGK

SEQ ID NO: 472 - protein, 9 aa, Homo sapiens
VVGADGVGK

SEQ ID NO: 473 - protein, 9 aa, synthetic construct
VVKADGVGK

SEQ ID NO: 474 - protein, 9 aa, synthetic construct
KVGADGVGK

SEQ ID NO: 475 - protein, 9 aa, synthetic construct
VTGADGVGK

SEQ ID NO: 476 - protein, 9 aa, synthetic construct
VVGADGPGK

SEQ ID NO: 477 - protein, 9 aa, synthetic construct
KVKADGVGK

SEQ ID NO: 478 - protein, 9 aa, synthetic construct
KTGADGVGK

SEQ ID NO: 479 - protein, 10 aa, Homo sapiens
VVVGAAGVGK

SEQ ID NO: 480 - protein, 10 aa, synthetic construct
RVVGAAGVGK

SEQ ID NO: 481 - protein, 10 aa, synthetic construct
VVKGAAGVGK

SEQ ID NO: 482 - protein, 10 aa, synthetic construct
VTVGAAGVGK

SEQ ID NO: 483 - protein, 10 aa, synthetic construct
VVVGAAGPGK

SEQ ID NO: 484 - protein, 10 aa, synthetic construct
VVVGAARVGK

SEQ ID NO: 485 - protein, 10 aa, synthetic construct
KVVGAAGVGK

SEQ ID NO: 486 - protein, 9 aa, Homo sapiens
VVGAAGVGK

SEQ ID NO: 487 - protein, 9 aa, synthetic construct
VVKAAGVGK

SEQ ID NO: 488 - protein, 9 aa, synthetic construct
KVGAAGVGK

SEQ ID NO: 489 - protein, 9 aa, synthetic construct
VTGAAGVGK

SEQ ID NO: 490 - protein, 9 aa, synthetic construct
VVGAAGPGK

SEQ ID NO: 491 - protein, 9 aa, synthetic construct
KVKAAGVGK

SEQ ID NO: 492 - protein, 9 aa, synthetic construct
KTGAAGVGK

SEQ ID NO: 493 - protein, 10 aa, Homo sapiens
VVVGACGVGK

SEQ ID NO: 494 - protein, 10 aa, synthetic construct
RVVGACGVGK

SEQ ID NO: 495 - protein, 10 aa, synthetic construct
VVKGACGVGK

SEQ ID NO: 496 - protein, 10 aa, synthetic construct
VTVGACGVGK

SEQ ID NO: 497 - protein, 10 aa, synthetic construct
VVVGACGPGK

SEQ ID NO: 498 - protein, 10 aa, synthetic construct
VVVGACRVGK

SEQ ID NO: 499 - protein, 10 aa, synthetic construct
KVVGACGVGK

SEQ ID NO: 500 - protein, 9 aa, Homo sapiens
VVGACGVGK

SEQ ID NO: 501 - protein, 9 aa, synthetic construct
VVKACGVGK

SEQ ID NO: 502 - protein, 9 aa, synthetic construct
KVGACGVGK

SEQ ID NO: 503 - protein, 9 aa, synthetic construct
VTGACGVGK

SEQ ID NO: 504 - protein, 9 aa, synthetic construct
VVGACGPGK

SEQ ID NO: 505 - protein, 9 aa, synthetic construct
KVKACGVGK

SEQ ID NO: 506 - protein, 9 aa, synthetic construct
KTGACGVGK

SEQ ID NO: 507 - protein, 10 aa, Homo sapiens
VVVGACGVGK

SEQ ID NO: 508 - protein, 10 aa, synthetic construct
EVVGACGVGK

SEQ ID NO: 509 - protein, 10 aa, synthetic construct
VTVGACGVGK

SEQ ID NO: 510 - protein, 10 aa, synthetic construct
ETVGACGVGK

SEQ ID NO: 511 - protein, 10 aa, synthetic construct
VVVGACGVGR

SEQ ID NO: 512 - protein, 10 aa, synthetic construct
EVVGACGVGR

SEQ ID NO: 513 - protein, 9 aa, Homo sapiens
VVGAVGVGK

SEQ ID NO: 514 - protein, 9 aa, synthetic construct
VTGAVGVGK

SEQ ID NO: 515 - protein, 9 aa, synthetic construct
VVGAVGVGR

SEQ ID NO: 516 - protein, 9 aa, synthetic construct
VTGAVGVGR

SEQ ID NO: 517 - protein, 9 aa, synthetic construct
VSGAVGVGK

SEQ ID NO: 518 - protein, 9 aa, synthetic construct
VLGAVGVGK

SEQ ID NO: 519 - protein, 10 aa, Homo sapiens
VVVGADGVGK

SEQ ID NO: 520 - protein, 10 aa, synthetic construct
EVVGADGVGK

SEQ ID NO: 521 - protein, 10 aa, synthetic construct
VTVGADGVGK

SEQ ID NO: 522 - protein, 10 aa, synthetic construct
ETVGADGVGK

SEQ ID NO: 523 - protein, 10 aa, synthetic construct
VVVGADGVGR

SEQ ID NO: 524 - protein, 10 aa, synthetic construct
EVVGADGVGR

SEQ ID NO: 525 - protein, 10 aa, Homo sapiens
VVVGARGVGK

SEQ ID NO: 526 - protein, 10 aa, synthetic construct
EVVGARGVGK

SEQ ID NO: 527 - protein, 10 aa, synthetic construct
VTVGARGVGK

SEQ ID NO: 528 - protein, 10 aa, synthetic construct
ETVGARGVGK

SEQ ID NO: 529 - protein, 10 aa, synthetic construct
VVVGARGVGR

SEQ ID NO: 530 - protein, 10 aa, synthetic construct
EVVGARGVGR

SEQ ID NO: 531 - protein, 10 aa, Homo sapiens
VVVGAVGVGK

SEQ ID NO: 532 - protein, 10 aa, synthetic construct
EVVGAVGVGK

SEQ ID NO: 533 - protein, 10 aa, synthetic construct
VTVGAVGVGK

SEQ ID NO: 534 - protein, 10 aa, synthetic construct
ETVGAVGVGK

SEQ ID NO: 535 - protein, 10 aa, synthetic construct
VVVGAVGVGR

SEQ ID NO: 536 - protein, 10 aa, synthetic construct
EVVGAVGVGR

SEQ ID NO: 537 - protein, 10 aa, Homo sapiens
VVVGAAGVGK

SEQ ID NO: 538 - protein, 10 aa, synthetic construct
EVVGAAGVGK

SEQ ID NO: 539 - protein, 10 aa, synthetic construct
VTVGAAGVGK

SEQ ID NO: 540 - protein, 10 aa, synthetic construct
ETVGAAGVGK

SEQ ID NO: 541 - protein, 10 aa, synthetic construct
VVVGAAGVGR

SEQ ID NO: 542 - protein, 10 aa, synthetic construct
EVVGAAGVGR

SEQ ID NO: 543 - protein, 9 aa, Homo sapiens
VVGARGVGK

SEQ ID NO: 544 - protein, 9 aa, synthetic construct
VTGARGVGK

SEQ ID NO: 545 - protein, 9 aa, synthetic construct
VVGARGVGR

SEQ ID NO: 546 - protein, 9 aa, synthetic construct
VTGARGVGR

SEQ ID NO: 547 - protein, 9 aa, synthetic construct
VSGARGVGK

SEQ ID NO: 548 - protein, 9 aa, synthetic construct
VLGARGVGK

SEQ ID NO: 549 - protein, 9 aa, Homo sapiens
VVGADGVGK

SEQ ID NO: 550 - protein, 9 aa, synthetic construct
VTGADGVGK

SEQ ID NO: 551 - protein, 9 aa, synthetic construct
VVGADGVGR

SEQ ID NO: 552 - protein, 9 aa, synthetic construct
VTGADGVGR

SEQ ID NO: 553 - protein, 9 aa, synthetic construct
VSGADGVGK

SEQ ID NO: 554 - protein, 9 aa, synthetic construct
VLGADGVGK

SEQ ID NO: 555 - protein, 9 aa, Homo sapiens
VVGAAGVGK

SEQ ID NO: 556 - protein, 9 aa, synthetic construct
VTGAAGVGK

SEQ ID NO: 557 - protein, 9 aa, synthetic construct
VVGAAGVGR

SEQ ID NO: 558 - protein, 9 aa, synthetic construct
VTGAAGVGR

SEQ ID NO: 559 - protein, 9 aa, synthetic construct
VSGAAGVGK

SEQ ID NO: 560 - protein, 9 aa, synthetic construct
VLGAAGVGK

SEQ ID NO: 561 - protein, 9 aa, Homo sapiens
VVGACGVGK

SEQ ID NO: 562 - protein, 9 aa, synthetic construct
VTGACGVGK

SEQ ID NO: 563 - protein, 9 aa, synthetic construct
VVGACGVGR

SEQ ID NO: 564 - protein, 9 aa, synthetic construct
VTGACGVGR

SEQ ID NO: 565 - protein, 9 aa, synthetic construct
VSGACGVGK

SEQ ID NO: 566 - protein, 9 aa, synthetic construct
VLGACGVGK

SEQ ID NO: 567 - protein, 10 aa, Homo sapiens
GADGVGKSAL

SEQ ID NO: 568 - protein, 10 aa, synthetic construct
GPDGVGKSAL

SEQ ID NO: 569 - protein, 10 aa, synthetic construct
AADGVGKSAL

SEQ ID NO: 570 - protein, 10 aa, synthetic construct
SADGVGKSAL

SEQ ID NO: 571 - protein, 10 aa, synthetic construct
APDGVGKSAL

SEQ ID NO: 572 - protein, 10 aa, synthetic construct
SPDPVGKSAL

SEQ ID NO: 573 - protein, 10 aa, synthetic construct
GADPVGKSAL

SEQ ID NO: 574 - protein, 10 aa, Homo sapiens
GARGVGKSAL

SEQ ID NO: 575 - protein, 10 aa, synthetic construct
GPRGVGKSAL

SEQ ID NO: 576 - protein, 10 aa, synthetic construct
AARGVGKSAL

SEQ ID NO: 577 - protein, 10 aa, synthetic construct
SARGVGKSAL

SEQ ID NO: 578 - protein, 10 aa, synthetic construct
APRGVGKSAL

SEQ ID NO: 579 - protein, 10 aa, synthetic construct
SPRPVGKSAL

SEQ ID NO: 580 - protein, 10 aa, synthetic construct
GARPVGKSAL

SEQ ID NO: 581 - protein, 10 aa, Homo sapiens
GAAGVGKSAL

SEQ ID NO: 582 - protein, 10 aa, synthetic construct
GPAGVGKSAL

SEQ ID NO: 583 - protein, 10 aa, synthetic construct
AAAGVGKSAL

SEQ ID NO: 584 - protein, 10 aa, synthetic construct
SAAGVGKSAL

SEQ ID NO: 585 - protein, 10 aa, synthetic construct
APAGVGKSAL

SEQ ID NO: 586 - protein, 10 aa, synthetic construct
SPAPVGKSAL

SEQ ID NO: 587 - protein, 10 aa, synthetic construct
GAAPVGKSAL

SEQ ID NO: 588 - protein, 10 aa, Homo sapiens
GACGVGKSAL

SEQ ID NO: 589 - protein, 10 aa, synthetic construct
GPCGVGKSAL

SEQ ID NO: 590 - protein, 10 aa, synthetic construct
AACGVGKSAL

SEQ ID NO: 591 - protein, 10 aa, synthetic construct
SACGVGKSAL

SEQ ID NO: 592 - protein, 10 aa, synthetic construct
APCGVGKSAL

SEQ ID NO: 593 - protein, 10 aa, synthetic construct
SPCPVGKSAL

SEQ ID NO: 594 - protein, 10 aa, synthetic construct
GACPVGKSAL

SEQ ID NO: 595 - protein, 10 aa, Homo sapiens
GAVGVGKSAL

SEQ ID NO: 596 - protein, 10 aa, synthetic construct
GPVGVGKSAL

SEQ ID NO: 597 - protein, 10 aa, synthetic construct
AAVGVGKSAL

SEQ ID NO: 598 - protein, 10 aa, synthetic construct
SAVGVGKSAL

SEQ ID NO: 599 - protein, 10 aa, synthetic construct
APVGVGKSAL

SEQ ID NO: 600 - protein, 10 aa, synthetic construct
SPVPVGKSAL

SEQ ID NO: 601 - protein, 10 aa, synthetic construct
GAVPVGKSAL

SEQ ID NO: 602 - protein, 9 aa, Homo sapiens
AVGVGKSAL

SEQ ID NO: 603 - protein, 9 aa, synthetic construct
AVPVGKSAL

SEQ ID NO: 604 - protein, 9 aa, synthetic construct
AVGVGKSSL

SEQ ID NO: 605 - protein, 9 aa, synthetic construct
SVGVGKSAL

SEQ ID NO: 606 - protein, 9 aa, synthetic construct
AVGVGKSEL

SEQ ID NO: 607 - protein, 9 aa, synthetic construct
AVAVGKSAL

SEQ ID NO: 608 - protein, 9 aa, Homo sapiens
ARGVGKSAL

SEQ ID NO: 609 - protein, 9 aa, synthetic construct
ARPVGKSAL

SEQ ID NO: 610 - protein, 9 aa, synthetic construct
ARGVGKSSL

SEQ ID NO: 611 - protein, 9 aa, synthetic construct
SRGVGKSAL

SEQ ID NO: 612 - protein, 9 aa, synthetic construct
ARGVGKSEL

SEQ ID NO: 613 - protein, 9 aa, synthetic construct
ARAVGKSAL

SEQ ID NO: 614 - protein, 9 aa, Homo sapiens
ADGVGKSAL

SEQ ID NO: 615 - protein, 9 aa, synthetic construct
ADPVGKSAL

SEQ ID NO: 616 - protein, 9 aa, synthetic construct
ADGVGKSSL

SEQ ID NO: 617 - protein, 9 aa, synthetic construct
SDGVGKSAL

SEQ ID NO: 618 - protein, 9 aa, synthetic construct
ADGVGKSEL

SEQ ID NO: 619 - protein, 9 aa, synthetic construct
ADAVGKSAL

SEQ ID NO: 620 - protein, 9 aa, Homo sapiens
AAGVGKSAL

SEQ ID NO: 621 - protein, 9 aa, synthetic construct
AAPVGKSAL

SEQ ID NO: 622 - protein, 9 aa, synthetic construct
AAGVGKSSL

SEQ ID NO: 623 - protein, 9 aa, synthetic construct
SAGVGKSAL

SEQ ID NO: 624 - protein, 9 aa, synthetic construct
AAGVGKSEL

SEQ ID NO: 625 - protein, 9 aa, synthetic construct
AAAVGKSAL

SEQ ID NO: 626 - protein, 9 aa, Homo sapiens
ACGVGKSAL

SEQ ID NO: 627 - protein, 9 aa, synthetic construct
ACPVGKSAL

SEQ ID NO: 628 - protein, 9 aa, synthetic construct
ACGVGKSSL

SEQ ID NO: 629 - protein, 9 aa, synthetic construct
SCGVGKSAL

SEQ ID NO: 630 - protein, 9 aa, synthetic construct
ACGVGKSEL

SEQ ID NO: 631 - protein, 9 aa, synthetic construct
ACAVGKSAL

SEQ ID NO: 632 - protein, 10 aa, Homo sapiens
GADGVGKSAL

SEQ ID NO: 633 - protein, 10 aa, synthetic construct
GADGVGKSAL

SEQ ID NO: 634 - protein, 10 aa, synthetic construct
GVDGVGKSAL

SEQ ID NO: 635 - protein, 10 aa, synthetic construct
VADGVGKSAL

SEQ ID NO: 636 - protein, 10 aa, synthetic construct
GADGVGKSSL

SEQ ID NO: 637 - protein, 10 aa, synthetic construct
VADGVGKSSL

SEQ ID NO: 638 - protein, 10 aa, synthetic construct
GADGVGKSEL

SEQ ID NO: 639 - protein, 9 aa, Homo sapiens
GADGVGKSA

SEQ ID NO: 640 - protein, 9 aa, synthetic construct
GADGVGKSL

SEQ ID NO: 641 - protein, 9 aa, synthetic construct
GADGVGKSV

SEQ ID NO: 642 - protein, 9 aa, synthetic construct
GADGVGKSF

SEQ ID NO: 643 - protein, 9 aa, synthetic construct
GSDGVGKSA

SEQ ID NO: 644 - protein, 9 aa, synthetic construct
VADGVGKSL

SEQ ID NO: 645 - protein, 10 aa, Homo sapiens
GAVGVGKSAL

SEQ ID NO: 646 - protein, 10 aa, synthetic construct
GAVGVGKSAL

SEQ ID NO: 647 - protein, 10 aa, synthetic construct
GVVGVGKSAL

SEQ ID NO: 648 - protein, 10 aa, synthetic construct
VAVGVGKSAL

SEQ ID NO: 649 - protein, 10 aa, synthetic construct
GAVGVGKSSL

SEQ ID NO: 650 - protein, 10 aa, synthetic construct
VAVGVGKSSL

SEQ ID NO: 651 - protein, 10 aa, synthetic construct
GAVGVGKSEL

SEQ ID NO: 652 - protein, 9 aa, Homo sapiens
GAVGVGKSA

SEQ ID NO: 653 - protein, 9 aa, synthetic construct
GAVGVGKSL

SEQ ID NO: 654 - protein, 9 aa, synthetic construct
GAVGVGKSV

SEQ ID NO: 655 - protein, 9 aa, synthetic construct
GAVGVGKSF

SEQ ID NO: 656 - protein, 9 aa, synthetic construct
GSVGVGKSA

SEQ ID NO: 657 - protein, 9 aa, synthetic construct
VAVGVGKSL

SEQ ID NO: 658 - protein, 10 aa, Homo sapiens
GARGVGKSAL

SEQ ID NO: 659 - protein, 10 aa, synthetic construct
GARGVGKSAL

SEQ ID NO: 660 - protein, 10 aa, synthetic construct
GVRGVGKSAL

SEQ ID NO: 661 - protein, 10 aa, synthetic construct
VARGVGKSAL

SEQ ID NO: 662 - protein, 10 aa, synthetic construct
GARGVGKSSL

SEQ ID NO: 663 - protein, 10 aa, synthetic construct
VARGVGKSSL

SEQ ID NO: 664 - protein, 10 aa, synthetic construct
GARGVGKSEL

SEQ ID NO: 665 - protein, 9 aa, Homo sapiens
GARGVGKSA

SEQ ID NO: 666 - protein, 9 aa, synthetic construct
GARGVGKSL

SEQ ID NO: 667 - protein, 9 aa, synthetic construct
GARGVGKSV

SEQ ID NO: 668 - protein, 9 aa, synthetic construct
GARGVGKSF

SEQ ID NO: 669 - protein, 9 aa, synthetic construct
GSRGVGKSA

SEQ ID NO: 670 - protein, 9 aa, synthetic construct
VARGVGKSL

SEQ ID NO: 671 - protein, 10 aa, Homo sapiens
GAAGVGKSAL

SEQ ID NO: 672 - protein, 10 aa, synthetic construct
GAAGVGKSAL

SEQ ID NO: 673 - protein, 10 aa, synthetic construct
GVAGVGKSAL

SEQ ID NO: 674 - protein, 10 aa, synthetic construct
VAAGVGKSAL

SEQ ID NO: 675 - protein, 10 aa, synthetic construct
GAAGVGKSSL

SEQ ID NO: 676 - protein, 10 aa, synthetic construct
VAAGVGKSSL

SEQ ID NO: 677 - protein, 10 aa, synthetic construct
GAAGVGKSEL

SEQ ID NO: 678 - protein, 9 aa, Homo sapiens
GAAGVGKSA

SEQ ID NO: 679 - protein, 9 aa, synthetic construct
GAAGVGKSL

SEQ ID NO: 680 - protein, 9 aa, synthetic construct
GAAGVGKSV

SEQ ID NO: 681 - protein, 9 aa, synthetic construct
GAAGVGKSF

SEQ ID NO: 682 - protein, 9 aa, synthetic construct
GSAGVGKSA

SEQ ID NO: 683 - protein, 9 aa, synthetic construct
VAAGVGKSL

SEQ ID NO: 684 - protein, 10 aa, Homo sapiens
GACGVGKSAL

SEQ ID NO: 685 - protein, 10 aa, synthetic construct
GACGVGKSAL

SEQ ID NO: 686 - protein, 10 aa, synthetic construct
GVCGVGKSAL

SEQ ID NO: 687 - protein, 10 aa, synthetic construct
VACGVGKSAL

SEQ ID NO: 688 - protein, 10 aa, synthetic construct
GACGVGKSSL

SEQ ID NO: 689 - protein, 10 aa, synthetic construct
VACGVGKSSL

SEQ ID NO: 690 - protein, 10 aa, synthetic construct
GACGVGKSEL

SEQ ID NO: 691 - protein, 9 aa, Homo sapiens
GACGVGKSA

SEQ ID NO: 692 - protein, 9 aa, synthetic construct
GACGVGKSL

SEQ ID NO: 693 - protein, 9 aa, synthetic construct
GACGVGKSV

SEQ ID NO: 694 - protein, 9 aa, synthetic construct
GACGVGKSF

SEQ ID NO: 695 - protein, 9 aa, synthetic construct
GSCGVGKSA

SEQ ID NO: 696 - protein, 9 aa, synthetic construct
VACGVGKSL

SEQ ID NO: 697 - protein, 10 aa, Homo sapiens
GADGVGKSAL

SEQ ID NO: 698 - protein, 10 aa, synthetic construct
GFDGVGKSAL

SEQ ID NO: 699 - protein, 10 aa, synthetic construct
GADGVGKSAF

SEQ ID NO: 700 - protein, 10 aa, synthetic construct
GYDGVGKSAL

SEQ ID NO: 701 - protein, 10 aa, synthetic construct
GFDGVGKSAF

SEQ ID NO: 702 - protein, 10 aa, synthetic construct
GYDGVGKSAF

SEQ ID NO: 703 - protein, 10 aa, synthetic construct
VFDGVGKSAL

SEQ ID NO: 704 - protein, 9 aa, Homo sapiens
GADGVGKSA

SEQ ID NO: 705 - protein, 9 aa, synthetic construct
GFDGVGKSA

SEQ ID NO: 706 - protein, 9 aa, synthetic construct
GADGVGKSL

SEQ ID NO: 707 - protein, 9 aa, synthetic construct
GYDGVGKSA

SEQ ID NO: 708 - protein, 9 aa, synthetic construct
GADGVGKSF

SEQ ID NO: 709 - protein, 9 aa, synthetic construct
GYDGVGKSL

SEQ ID NO: 710 - protein, 9 aa, synthetic construct
GYDGVGKSF

SEQ ID NO: 711 - protein, 9 aa, synthetic construct
GFDGVGKSL

SEQ ID NO: 712 - protein, 10 aa, Homo sapiens
GAVGVGKSAL

SEQ ID NO: 713 - protein, 10 aa, synthetic construct
GFVGVGKSAL

SEQ ID NO: 714 - protein, 10 aa, synthetic construct
GAVGVGKSAF

SEQ ID NO: 715 - protein, 10 aa, synthetic construct
GYVGVGKSAL

SEQ ID NO: 716 - protein, 10 aa, synthetic construct
GFVGVGKSAF

SEQ ID NO: 717 - protein, 10 aa, synthetic construct
GYVGVGKSAF

SEQ ID NO: 718 - protein, 10 aa, synthetic construct
VFVGVGKSAL

SEQ ID NO: 719 - protein, 9 aa, Homo sapiens
GAVGVGKSA

SEQ ID NO: 720 - protein, 9 aa, synthetic construct
GFVGVGKSA

SEQ ID NO: 721 - protein, 9 aa, synthetic construct
GAVGVGKSL

SEQ ID NO: 722 - protein, 9 aa, synthetic construct
GYVGVGKSA

SEQ ID NO: 723 - protein, 9 aa, synthetic construct
GAVGVGKSF

SEQ ID NO: 724 - protein, 9 aa, synthetic construct
GYVGVGKSL

SEQ ID NO: 725 - protein, 9 aa, synthetic construct
GYVGVGKSF

SEQ ID NO: 726 - protein, 9 aa, synthetic construct
GFVGVGKSL

SEQ ID NO: 727 - protein, 10 aa, Homo sapiens
GARGVGKSAL

SEQ ID NO: 728 - protein, 10 aa, synthetic construct
GFRGVGKSAL

SEQ ID NO: 729 - protein, 10 aa, synthetic construct
GARGVGKSAF

SEQ ID NO: 730 - protein, 10 aa, synthetic construct
GYRGVGKSAL

SEQ ID NO: 731 - protein, 10 aa, synthetic construct
GFRGVGKSAF

SEQ ID NO: 732 - protein, 10 aa, synthetic construct
GYRGVGKSAF

SEQ ID NO: 733 - protein, 10 aa, synthetic construct
VFRGVGKSAL

SEQ ID NO: 734 - protein, 9 aa, Homo sapiens
GARGVGKSA

SEQ ID NO: 735 - protein, 9 aa, synthetic construct
GFRGVGKSA

SEQ ID NO: 736 - protein, 9 aa, synthetic construct
GARGVGKSL

SEQ ID NO: 737 - protein, 9 aa, synthetic construct
GYRGVGKSA

SEQ ID NO: 738 - protein, 9 aa, synthetic construct
GARGVGKSF

SEQ ID NO: 739 - protein, 9 aa, synthetic construct
GYRGVGKSL

SEQ ID NO: 740 - protein, 9 aa, synthetic construct
GYRGVGKSF

SEQ ID NO: 741 - protein, 9 aa, synthetic construct
GFRGVGKSL

SEQ ID NO: 742 - protein, 10 aa, Homo sapiens
GAAGVGKSAL

SEQ ID NO: 743 - protein, 10 aa, synthetic construct
GFAGVGKSAL

SEQ ID NO: 744 - protein, 10 aa, synthetic construct
GAAGVGKSAF

SEQ ID NO: 745 - protein, 10 aa, synthetic construct
GYAGVGKSAL

SEQ ID NO: 746 - protein, 10 aa, synthetic construct
GFAGVGKSAF

SEQ ID NO: 747 - protein, 10 aa, synthetic construct
GYAGVGKSAF

SEQ ID NO: 748 - protein, 10 aa, synthetic construct
VFAGVGKSAL

SEQ ID NO: 749 - protein, 9 aa, Homo sapiens
GAAGVGKSA

SEQ ID NO: 750 - protein, 9 aa, synthetic construct
GFAGVGKSA

SEQ ID NO: 751 - protein, 9 aa, synthetic construct
GAAGVGKSL

SEQ ID NO: 752 - protein, 9 aa, synthetic construct
GYAGVGKSA

SEQ ID NO: 753 - protein, 9 aa, synthetic construct
GAAGVGKSF

SEQ ID NO: 754 - protein, 9 aa, synthetic construct
GYAGVGKSL

SEQ ID NO: 755 - protein, 9 aa, synthetic construct
GYAGVGKSF

SEQ ID NO: 756 - protein, 9 aa, synthetic construct
GFAGVGKSL

SEQ ID NO: 757 - protein, 10 aa, Homo sapiens
GACGVGKSAL

SEQ ID NO: 758 - protein, 10 aa, synthetic construct
GFCGVGKSAL

SEQ ID NO: 759 - protein, 10 aa, synthetic construct
GACGVGKSAF

SEQ ID NO: 760 - protein, 10 aa, synthetic construct
GYCGVGKSAL

SEQ ID NO: 761 - protein, 10 aa, synthetic construct
GFCGVGKSAF

SEQ ID NO: 762 - protein, 10 aa, synthetic construct
GYCGVGKSAF

SEQ ID NO: 763 - protein, 10 aa, synthetic construct
VFCGVGKSAL

SEQ ID NO: 764 - protein, 9 aa, Homo sapiens
GACGVGKSA

SEQ ID NO: 765 - protein, 9 aa, synthetic construct
GFCGVGKSA

SEQ ID NO: 766 - protein, 9 aa, synthetic construct
GACGVGKSL

SEQ ID NO: 767 - protein, 9 aa, synthetic construct
GYCGVGKSA

SEQ ID NO: 768 - protein, 9 aa, synthetic construct
GACGVGKSF

SEQ ID NO: 769 - protein, 9 aa, synthetic construct
GYCGVGKSL

SEQ ID NO: 770 - protein, 9 aa, synthetic construct
GYCGVGKSF

SEQ ID NO: 771 - protein, 9 aa, synthetic construct
GFCGVGKSL

SEQ ID NO: 772 - protein, 10 aa, Homo sapiens
GAVGVGKSAL

SEQ ID NO: 773 - protein, 10 aa, synthetic construct
FAVGVGKSAL

SEQ ID NO: 774 - protein, 10 aa, synthetic construct
YAVGVGKSAL

SEQ ID NO: 775 - protein, 10 aa, synthetic construct
LAVGVGKSAL

SEQ ID NO: 776 - protein, 10 aa, synthetic construct
GAVPVGKSAL

SEQ ID NO: 777 - protein, 10 aa, synthetic construct
GVVGVGKSAL

SEQ ID NO: 778 - protein, 9 aa, Homo sapiens
GAVGVGKSA

SEQ ID NO: 779 - protein, 9 aa, synthetic construct
FAVGVGKSA

SEQ ID NO: 780 - protein, 9 aa, synthetic construct
YAVGVGKSA

SEQ ID NO: 781 - protein, 9 aa, synthetic construct
LAVGVGKSA

SEQ ID NO: 782 - protein, 9 aa, synthetic construct
GAVGVGKSL

SEQ ID NO: 783 - protein, 9 aa, synthetic construct
FAVGVGKSL

SEQ ID NO: 784 - protein, 10 aa, Homo sapiens
GARGVGKSAL

SEQ ID NO: 785 - protein, 10 aa, synthetic construct
FARGVGKSAL

SEQ ID NO: 786 - protein, 10 aa, synthetic construct
YARGVGKSAL

SEQ ID NO: 787 - protein, 10 aa, synthetic construct
LARGVGKSAL

SEQ ID NO: 788 - protein, 10 aa, synthetic construct
GARPVGKSAL

SEQ ID NO: 789 - protein, 10 aa, synthetic construct
GVRGVGKSAL

SEQ ID NO: 790 - protein, 9 aa, Homo sapiens
GARGVGKSA

SEQ ID NO: 791 - protein, 9 aa, synthetic construct
FARGVGKSA

SEQ ID NO: 792 - protein, 9 aa, synthetic construct
YARGVGKSA

SEQ ID NO: 793 - protein, 9 aa, synthetic construct
LARGVGKSA

SEQ ID NO: 794 - protein, 9 aa, synthetic construct
GARGVGKSL

SEQ ID NO: 795 - protein, 9 aa, synthetic construct
FARGVGKSL

SEQ ID NO: 796 - protein, 10 aa, Homo sapiens
GADGVGKSAL

SEQ ID NO: 797 - protein, 10 aa, synthetic construct
FADGVGKSAL

SEQ ID NO: 798 - protein, 10 aa, synthetic construct
YADGVGKSAL

SEQ ID NO: 799 - protein, 10 aa, synthetic construct
LADGVGKSAL

SEQ ID NO: 800 - protein, 10 aa, synthetic construct
GADPVGKSAL

SEQ ID NO: 801 - protein, 10 aa, synthetic construct
GVDGVGKSAL

SEQ ID NO: 802 - protein, 9 aa, Homo sapiens
GADGVGKSA

SEQ ID NO: 803 - protein, 9 aa, synthetic construct
FADGVGKSA

SEQ ID NO: 804 - protein, 9 aa, synthetic construct
YADGVGKSA

SEQ ID NO: 805 - protein, 9 aa, synthetic construct
LADGVGKSA

SEQ ID NO: 806 - protein, 9 aa, synthetic construct
GADGVGKSL

SEQ ID NO: 807 - protein, 9 aa, synthetic construct
FADGVGKSL

SEQ ID NO: 808 - protein, 10 aa, Homo sapiens
GAAGVGKSAL

SEQ ID NO: 809 - protein, 10 aa, synthetic construct
FAAGVGKSAL

SEQ ID NO: 810 - protein, 10 aa, synthetic construct
YAAGVGKSAL

SEQ ID NO: 811 - protein, 10 aa, synthetic construct
LAAGVGKSAL

SEQ ID NO: 812 - protein, 10 aa, synthetic construct
GAAPVGKSAL

SEQ ID NO: 813 - protein, 10 aa, synthetic construct
GVAGVGKSAL

SEQ ID NO: 814 - protein, 9 aa, Homo sapiens
GAAGVGKSA

SEQ ID NO: 815 - protein, 9 aa, synthetic construct
FAAGVGKSA

SEQ ID NO: 816 - protein, 9 aa, synthetic construct
YAAGVGKSA

SEQ ID NO: 817 - protein, 9 aa, synthetic construct
LAAGVGKSA

SEQ ID NO: 818 - protein, 9 aa, synthetic construct
GAAGVGKSL

SEQ ID NO: 819 - protein, 9 aa, synthetic construct
FAAGVGKSL

SEQ ID NO: 820 - protein, 10 aa, Homo sapiens
GACGVGKSAL

SEQ ID NO: 821 - protein, 10 aa, synthetic construct
FACGVGKSAL

SEQ ID NO: 822 - protein, 10 aa, synthetic construct
YACGVGKSAL

SEQ ID NO: 823 - protein, 10 aa, synthetic construct
LACGVGKSAL

SEQ ID NO: 824 - protein, 10 aa, synthetic construct
GACPVGKSAL

SEQ ID NO: 825 - protein, 10 aa, synthetic construct
GVCGVGKSAL

SEQ ID NO: 826 - protein, 9 aa, Homo sapiens
GACGVGKSA

SEQ ID NO: 827 - protein, 9 aa, synthetic construct
FACGVGKSA

SEQ ID NO: 828 - protein, 9 aa, synthetic construct
YACGVGKSA

SEQ ID NO: 829 - protein, 9 aa, synthetic construct
LACGVGKSA

SEQ ID NO: 830 - protein, 9 aa, synthetic construct
GACGVGKSL

SEQ ID NO: 831 - protein, 9 aa, synthetic construct
FACGVGKSL

SEQ ID NO: 832 - protein, 21 aa, Homo sapiens
YKLVVVGADGVGKSALTIQLI

SEQ ID NO: 833 - protein, 21 aa, synthetic construct
YKLSVVGADGVGKSALTIQLI

SEQ ID NO: 834 - protein, 21 aa, synthetic construct
YKLAVVGADGVGKSALTIQLI

SEQ ID NO: 835 - protein, 21 aa, Homo sapiens
YKLVVVGACGVGKSALTIQLI

SEQ ID NO: 836 - protein, 21 aa, Homo sapiens
YKLVVVGAVGVGKSALTIQLI

SEQ ID NO: 837 - protein, 21 aa, Homo sapiens
YKLVVVGAAGVGKSALTIQLI

SEQ ID NO: 838 - protein, 21 aa, Homo sapiens
YKLVVVGARGVGKSALTIQLI

SEQ ID NO: 839 - protein, 6 aa, synthetic construct
HHHHHH

SEQ ID NO: 840 - protein, 9 aa, synthetic construct
GLDGVGKSA

SEQ ID NO: 841 - protein, 9 aa, synthetic construct
GADGVLKSA

SEQ ID NO: 842 - protein, 9 aa, synthetic construct
GLDGVGKSL

SEQ ID NO: 843 - protein, 9 aa, synthetic construct
GLDGVLKSL

SEQ ID NO: 844 - protein, 9 aa, synthetic construct
GLDGVGKSV

SEQ ID NO: 845 - protein, 9 aa, synthetic construct
GVDGVGKSL

SEQ ID NO: 846 - protein, 9 aa, synthetic construct
GADGVGKSY

SEQ ID NO: 847 - protein, 9 aa, synthetic construct
RADGVGKSY

SEQ ID NO: 848 - protein, 9 aa, synthetic construct
GLDGVGKSY

SEQ ID NO: 849 - protein, 9 aa, synthetic construct
GFDGVGKSF

SEQ ID NO: 850 - protein, 9 aa, synthetic construct
GTDGVGKSY

SEQ ID NO: 851 - protein, 9 aa, synthetic construct
GADGVGKSW

SEQ ID NO: 852 - protein, 9 aa, synthetic construct
GADGVGKSK

SEQ ID NO: 853 - protein, 9 aa, synthetic construct
GADGVGKSR

SEQ ID NO: 854 - protein, 9 aa, synthetic construct
DADGVGKSR

SEQ ID NO: 855 - protein, 9 aa, synthetic construct
GLDGVGKSW

SEQ ID NO: 856 - protein, 9 aa, synthetic construct
KLDGVGKSW

SEQ ID NO: 857 - protein, 9 aa, synthetic construct
GLDGVGKSK

SEQ ID NO: 858 - protein, 9 aa, synthetic construct
GLDGVGLSK

SEQ ID NO: 859 - protein, 9 aa, synthetic construct
GLDGVGKSR

SEQ ID NO: 860 - protein, 9 aa, synthetic construct
GLDGRGKSR

SEQ ID NO: 861 - protein, 9 aa, synthetic construct
GVDGVGKSK

SEQ ID NO: 862 - protein, 9 aa, synthetic construct
GVDGVGKSR

SEQ ID NO: 863 - protein, 9 aa, synthetic construct
EVDGVGKSR

SEQ ID NO: 864 - protein, 9 aa, synthetic construct
GEDGVGKSA

SEQ ID NO: 865 - protein, 9 aa, synthetic construct
GEDGVGKSL

SEQ ID NO: 866 - protein, 9 aa, synthetic construct
GEDGVGKSF

SEQ ID NO: 867 - protein, 9 aa, synthetic construct
GADGVGKSI

SEQ ID NO: 868 - protein, 9 aa, synthetic construct
GADGVGKSM

SEQ ID NO: 869 - protein, 9 aa, synthetic construct
GPDGVGKSA

SEQ ID NO: 870 - protein, 9 aa, synthetic construct
GPDGVGKSY

SEQ ID NO: 871 - protein, 9 aa, synthetic construct
GPDGVGKSF

SEQ ID NO: 872 - protein, 9 aa, synthetic construct
GPDGVGKSL

SEQ ID NO: 873 - protein, 9 aa, synthetic construct
GPDGVGKSV

SEQ ID NO: 874 - protein, 9 aa, synthetic construct
GPDGVGKSI

SEQ ID NO: 875 - protein, 9 aa, synthetic construct
GPDGVGKSM

SEQ ID NO: 876 - protein, 10 aa, synthetic construct
  VARIANT 6: note = D, V or C
VVVGAXGVGK

SEQ ID NO: 877 - protein, 10 aa, synthetic construct
  VARIANT 6: note = D, V or C
VLVGAXGVGK

SEQ ID NO: 878 - protein, 10 aa, synthetic construct
  VARIANT 6: note = D, V or C
RVVGAXGVGK

SEQ ID NO: 879 - protein, 10 aa, synthetic construct
  VARIANT 6: note = D, V or C
RLVGAXGVGK

SEQ ID NO: 880 - protein, 10 aa, synthetic construct
  VARIANT 6: note = D, V or C
VVVGAXLVGK

SEQ ID NO: 881 - protein, 10 aa, synthetic construct
  VARIANT 6: note = D, V or C
VLVGAXLVGK

SEQ ID NO: 882 - protein, 10 aa, synthetic construct
  VARIANT 6: note = D, V or C
RVVGAXLVGK

SEQ ID NO: 883 - protein, 10 aa, synthetic construct
  VARIANT 6: note = D, V or C
RLVGAXLVGK

SEQ ID NO: 884 - protein, 10 aa, synthetic construct
  VARIANT 6: note = D, V or C
KVVGAXGVGK

SEQ ID NO: 885 - protein, 10 aa, synthetic construct
  VARIANT 6: note = D, V or C
KLVGAXGVGK

SEQ ID NO: 886 - protein, 9 aa, synthetic construct
  VARIANT 5: note = R or V
VVGAXGVGK

SEQ ID NO: 887 - protein, 9 aa, synthetic construct
  VARIANT 5: note = R or V
VLGAXGVGK

SEQ ID NO: 888 - protein, 9 aa, synthetic construct
  VARIANT 5: note = R or V
RVGAXGVGK

SEQ ID NO: 889 - protein, 9 aa, synthetic construct
  VARIANT 5: note = R or V
VVGAXGLGK

SEQ ID NO: 890 - protein, 9 aa, synthetic construct
  VARIANT 5: note = R or V
VLGAXGLGK

SEQ ID NO: 891 - protein, 9 aa, synthetic construct
  VARIANT 5: note = R or V
RLGAXGLGK

SEQ ID NO: 892 - protein, 10 aa, synthetic construct
  VARIANT 6: note = D, R, V or C
VVVGAXGVGK

SEQ ID NO: 893 - protein, 10 aa, synthetic construct
  VARIANT 6: note = D, R, V or C
SVVGAXGVGK

SEQ ID NO: 894 - protein, 10 aa, synthetic construct
  VARIANT 6: note = D, R, V or C
VTVGAXGVGK

SEQ ID NO: 895 - protein, 10 aa, synthetic construct
  VARIANT 6: note = D, R, V or C
VSVGAXGVGK

SEQ ID NO: 896 - protein, 10 aa, synthetic construct
  VARIANT 6: note = D, R, V or C
AVVGAXGVGK

SEQ ID NO: 897 - protein, 10 aa, synthetic construct
  VARIANT 6: note = D, R, V or C
VVVGAXGLGK

SEQ ID NO: 898 - protein, 10 aa, synthetic construct
  VARIANT 6: note = D, R, V or C
STVGAXGVGK

SEQ ID NO: 899 - protein, 10 aa, synthetic construct
  VARIANT 6: note = D, R, V or C
ATVGAXGVGK

SEQ ID NO: 900 - protein, 9 aa, synthetic construct
  VARIANT 5: note = D, R or V
VVGAXGVGK

SEQ ID NO: 901 - protein, 9 aa, synthetic construct
  VARIANT 5: note = D, R or V
SVGAXGVGK

SEQ ID NO: 902 - protein, 9 aa, synthetic construct
  VARIANT 5: note = D, R or V
VTGAXGVGK

SEQ ID NO: 903 - protein, 9 aa, synthetic construct
  VARIANT 5: note = D, R or V
AVGAXGVGK

SEQ ID NO: 904 - protein, 9 aa, synthetic construct
  VARIANT 5: note = D, R or V
STGAXGVGK

SEQ ID NO: 905 - protein, 9 aa, synthetic construct
  VARIANT 5: note = D, R or V
ATGAXGVGK

SEQ ID NO: 906 - protein, 9 aa, synthetic construct
  VARIANT 5: note = D, R or V
VVGAXGLGK

SEQ ID NO: 907 - protein, 10 aa, synthetic construct
  VARIANT 6: note = R or V
VVVGAXGVGK

SEQ ID NO: 908 - protein, 10 aa, synthetic construct
  VARIANT 6: note = R or V
RVVGAXGVGK

SEQ ID NO: 909 - protein, 10 aa, synthetic construct
  VARIANT 6: note = R or V
VVKGAXGVGK

SEQ ID NO: 910 - protein, 10 aa, synthetic construct
  VARIANT 6: note = R or V
VTVGAXGVGK

SEQ ID NO: 911 - protein, 10 aa, synthetic construct
  VARIANT 6: note = R or V
VVVGAXGPGK

SEQ ID NO: 912 - protein, 10 aa, synthetic construct
  VARIANT 6: note = R or V
VVVGAXRVGK

SEQ ID NO: 913 - protein, 10 aa, synthetic construct
  VARIANT 6: note = R or V
KVVGAXGVGK

SEQ ID NO: 914 - protein, 9 aa, synthetic construct
  VARIANT 5: note = R or V
VVKAXGVGK

SEQ ID NO: 915 - protein, 9 aa, synthetic construct
  VARIANT 5: note = R or V
KVGAXGVGK

SEQ ID NO: 916 - protein, 9 aa, synthetic construct
  VARIANT 5: note = R or V
VTGAXGVGK

SEQ ID NO: 917 - protein, 9 aa, synthetic construct
  VARIANT 5: note = R or V
VVGAXGPGK

SEQ ID NO: 918 - protein, 9 aa, synthetic construct
  VARIANT 5: note = R or V
KVKAXGVGK

SEQ ID NO: 919 - protein, 9 aa, synthetic construct
  VARIANT 5: note = R or V
KTGAXGVGK

SEQ ID NO: 920 - protein, 10 aa, synthetic construct
  VARIANT 6: note = D, R, V or C
EVVGAXGVGK

SEQ ID NO: 921 - protein, 10 aa, synthetic construct
  VARIANT 6: note = D, R, V or C
ETVGAXGVGK

SEQ ID NO: 922 - protein, 10 aa, synthetic construct
  VARIANT 6: note = D, R, V or C
VVVGAXGVGR

SEQ ID NO: 923 - protein, 10 aa, synthetic construct
  VARIANT 6: note = D, R, V or C
EVVGAXGVGR

SEQ ID NO: 924 - protein, 10 aa, synthetic construct
  VARIANT 3: note = D or R
GAXGVGKSAL

SEQ ID NO: 925 - protein, 10 aa, synthetic construct
  VARIANT 3: note = D or R
GPXGVGKSAL

SEQ ID NO: 926 - protein, 10 aa, synthetic construct
  VARIANT 3: note = D or R
AAXGVGKSAL

SEQ ID NO: 927 - protein, 10 aa, synthetic construct
  VARIANT 3: note = D or R
SAXGVGKSAL

SEQ ID NO: 928 - protein, 10 aa, synthetic construct
  VARIANT 3: note = D or R
APXGVGKSAL

SEQ ID NO: 929 - protein, 10 aa, synthetic construct
  VARIANT 3: note = D or R
SPXPVGKSAL

SEQ ID NO: 930 - protein, 10 aa, synthetic construct
  VARIANT 3: note = D or R
GAXPVGKSAL

SEQ ID NO: 931 - protein, 10 aa, synthetic construct
GSDGVGKSAL

(19) United States
(12) Patent Application Publication
(10) Pub. No.: US 2026/0364202 A1
(43) Pub. Date: Nov. 19, 2026

(54) HETEROCLITIC NEOEPITOPE VACCINES

(71) Applicant: The Johns Hopkins University, Baltimore, MD (US)

(72) Inventors: Amanda Huff, Baltimore, MD (US); Elizabeth A. Jaffee, Baltimore, MD (US); Neeha Zaidi, Baltimore, MD (US); Mark Yarchoan, Baltimore, MD (US)

(21) Appl. No.: 19/358,130

(22) Filed: Oct. 14, 2025

Related U.S. Application Data
(63) Continuation of application No. PCT/US24/24542, filed on Apr. 14, 2024.
(60) Provisional application No. 63/459,442, filed on Apr. 14, 2023.

(51) Int. Cl. A61K 48/00; A61K 38/00; C07K 14/00; C12N 15/11; G16B 15/30; G16B 30/10

(52) U.S. Cl. CPC A61K 48/005; A61K 38/00; A61K 48/0083; A61K 48/0091; C07K 14/00; C12N 15/11; G16B 15/30; G16B 30/10

(57) ABSTRACT

Compositions of peptides modified to improve HLA binding or T cell recognition while conserving reactivity to the target neoepitope, enhance activation of neoantigen-specific T cells. Methods of use include treating cancer by administering one or more of these peptides generated by computer modeling.

### CROSS REFERENCE TO RELATED APPLICATIONS

This application is a continuation of International Application No. PCT/US24/24542, filed Apr. 14, 2024, which claims the benefit of priority under 35 U.S.C. § 119(e) to U.S. Provisional Application No. 63/459,442 filed Apr. 14, 2023, the entire contents of which applications are is incorporated herein by reference.

### STATEMENT REGARDING FEDERALLY SPONSORED RESEARCH

This invention was made with government support under grant nos. CA247886 and CA271470, awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted electronically in XML file format and is hereby incorporated by reference in its entirety. Said XML copy, created on Aug. 16, 2024, is named 348358_16002_SL.xml and is 826,942 bytes in size.”

### BACKGROUND

Many cancer neoantigens are poorly immunogenic due to weak binding of the neoepitope to human leukocyte antigen (HLA) thereby limiting presentation to cognate T cells. Modifications to the peptide amino acid sequence can improve HLA binding or T cell recognition while conserving reactivity to the target neoepitope, thereby enhancing activation of neoantigen-specific T cells. These modified epitopes, which improve antigen immunogenicity, are termed heteroclitic epitopes or altered peptide ligands (APLs).

### SUMMARY

Embodiments are directed to the designing and modelling of heteroclitic epitope vaccine candidates for diverse HLA subtypes. Accordingly, in certain aspects, a method of identifying heteroclitic neoepitopes comprises modeling peptides onto human leukocyte antigens (HLA), wherein the modeling comprises identifying positional sequence similarity of peptide epitopes that bind to one or a plurality of HLA haplotypes; selecting an HLA-peptide structure having a sequence identity greater than a control HLA-peptide structure; modeling the heteroclitic neoepitopes into an HLA cleft by introducing amino acid rotamers with the lowest energy confirmation at each position; validating the resulting HLA-peptide neoepitope complexes using a root-mean-square deviation analysis (Ca-RMSD); performing a high-resolution Monte Carlo simulation with minimization docking of the peptide to the HLA cleft to identify the lowest full-atom energy conformation; and, identifying heteroclitic neoepitopes. In certain embodiments, the modeling of the library of peptides onto human leukocyte antigens (HLA) comprises aligning peptide sequences with a consensus binding motif of HLA molecules. In certain embodiments, the consensus binding motif for 8-mer, 9-mer, 11-mer, 12-mer, 13-mer, 14-mer, 15-mer, 16-mer, 17-mer, 18-mer, 19-mer, 20-mer, 21-mer, 22-mer, 23-mer, 24-mer, or 25-mer epitopes comprises distinct anchor residue binding motifs subdivided into hydrophobic, hydrophilic, or neutral binding anchors. In certain embodiments, the root-mean-square deviation analysis is conducted to identify any unstable structural changes of an HLA cleft. In certain embodiments, anchor energetic-optimized structures are selected to identify anchor residue modifications. In certain embodiments, peptides are generated comprising combinations of anchor residue amino acid for each HLA class and subgroups thereof. In certain embodiments, the HLA-types comprise class I, II and subgroups thereof. In certain embodiments, the peptides are derived from tumor cells. In certain embodiments, the peptides comprise tumor antigens. In some embodiments, a heteroclitic epitope vaccine candidate is selected based on the assessment of one or more of contacts between the peptide and HLA cleft residues, solvent-assessable surface area (SASA), peptide rigidity as a measure of CaRMSD of the top 10 predicted structural models for each peptide-HLA complex, surface hydrophobicity, electrostatic potential and/or induction of the greatest T cell expansion and activation as compared to parental peptides.

Disclosed herein are method of generating and identifying heteroclitic peptides. In some embodiments, the method comprises aligning peptide sequences on to a parental sequence. In some embodiments, a parental sequence can be a control sequence. In some embodiments, the method comprises identifying a sequence having high homology to the epitope of a parental sequence. In some embodiments, the method comprises introducing amino acid rotamers with the lowest energy confirmation to the sequence having high homology to the epitope of the parental sequence. In some embodiments, the method further comprises quantifying differences in structural features the parental sequence and the sequence having high homology to the epitope of the parental sequence. In some embodiments, the feature comprises contacts between the peptide and HLA cleft residues, solvent-assessable surface area (SASA), peptide rigidity as a measure of CaRMSD of the top 10 predicted structural models for each peptide-HLA complex, surface hydrophobicity, and/or electrostatic potential. In some embodiments, the method further comprises in vitro co-culturing of the sequence having high homology with HLA-matched lymphocytes to validate which heteroclitic peptides induce the greatest T cell expansion and activation as compared to parental peptides, as assessed by IFN-gamma ELISpot assay. In some embodiment, the method further comprises selecting/identifying the heteroclitic peptide. In some embodiments, the heteroclitic peptide comprises at least a 75%, 85%, 90%, 95%, 97%, 98%, 99% sequence identity to any one or more of SEQ ID NOs: 1-838. In some embodiments, the heteroclitic peptide comprises at least a 75%, 85%, 90%, 95%, 97%, 98%, 99% sequence identity to any one or more of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. In certain embodiments, the heteroclitic peptide comprise any one or more of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. In certain embodiments, the heteroclitic peptide comprises an amino acid sequence having at least 95% sequence identity to any one or more of SEQ ID NOs: 1-838 having one or more conservative substitutions. In some embodiments, the heteroclitic peptide comprises the amino acid sequence of SEQ ID NO: 2, SEQ ID NO: 3, SEQ ID NO: 833, or SEQ ID NO: 834.

In another aspect, a method of generating a library of immunogenic peptides, comprises modeling a peptide onto human leukocyte antigens (HLA), wherein the modeling comprises identifying positional sequence similarity of peptide epitopes that bind to one or a plurality of HLA haplotypes; selecting an HLA-peptide structure having a sequence identity greater than a control HLA-peptide structure; modeling the heteroclitic neoepitopes into an HLA cleft by introducing amino acid rotamers with the lowest energy confirmation at each position; validating the resulting HLA-peptide complexes using a root-mean-square deviation analysis (Ca-RMSD); performing a high-resolution Monte Carlo simulation with minimization docking of the peptide to the HLA cleft to identify the lowest full-atom energy conformation; and, generating the library of immunogenic peptides. In certain embodiments, the peptides comprise peptides isolated from tumor cells. In certain embodiments, the peptides comprise tumor antigens. In certain embodiments, the tumor antigen is wherein the tumor antigen is a Kirsten rat sarcoma viral (KRAS) tumor antigen. In certain embodiments, the KRAS tumor antigen comprises one or more mutations. In certain embodiments, the KRAS peptide sequence is positionally aligned with 8-mer, 9-mer, 11-mer, 12-mer, 13-mer, 14-mer, 15-mer, 16-mer, 17-mer, 18-mer, 19-mer, 20-mer, 21-mer, 22-mer, 23-mer, 24-mer, or 25-mer epitopes that bind HLA molecules. In certain embodiments, the 8-mer, 9-mer, 11-mer, 12-mer, 13-mer, 14-mer, 15-mer, 16-mer, 17-mer, 18-mer, 19-mer, 20-mer 21-mer, 22-mer, 23-mer, 24-mer, or 25-mer epitopes comprises distinct anchor residue binding motifs subdivided into hydrophobic, hydrophilic, or neutral binding anchors. In certain embodiments, the HLA-KRAS peptide comprising the greatest sequence similarity is selected for modeling into an HLA cleft. In certain embodiments, the modeling into an HLA cleft comprises introducing amino acid rotamers with the lowest energy confirmation at each position. In certain embodiments, the root-mean-square deviation analysis is conducted to identify any unstable structural changes of an HLA cleft. In certain embodiments, anchor energetic-optimized HLA-KRAS structures are selected to identify anchor residue modifications. In certain embodiments, a library of KRAS peptides is generated comprising combinations of anchor residue amino acid for each HLA class and subgroups thereof. In certain embodiments, the HLA-types comprise class I, II and subgroups thereof. In certain embodiments, the KRAS peptides comprise heteroclitic epitopes. In some embodiments, an immunogenic peptide is selected based on the assessment of one or more of contacts between the peptide and HLA cleft residues, solvent-assessable surface area (SASA), peptide rigidity as a measure of CaRMSD of the top 10 predicted structural models for each peptide-HLA complex, surface hydrophobicity, electrostatic potential and/or induction of the greatest T cell expansion and activation as compared to parental peptides.

In certain embodiments, the heteroclitic peptide sequence is positionally aligned with epitopes that bind HLA molecules. In some embodiments, the epitopes are 8-mer epitopes, 9-mer epitopes, 10-mer epitopes, 11-mer epitopes, 12-mer epitopes, 13-mer epitopes, 14-mer epitopes, 15-mer epitopes, 16-mer epitopes, 17-mer epitopes, 18-mer epitopes, 19-mer epitopes, 20-mer epitopes, 21-mer epitopes, 22-mer epitopes, 23-mer epitopes, 24-mer epitopes, or 25-mer epitopes.

In another aspect, a method of treating cancer comprises administering to subject in need thereof, a therapeutically effective amount of one or more peptides comprising one or more heteroclitic epitopes.

In another aspect, a cancer vaccine comprises one or more peptides, wherein the peptides comprise at least one heteroclitic neoepitope. In certain embodiments, the peptide is a tumor antigen. In certain embodiments, the tumor antigen comprises a Kirsten rat sarcoma viral (KRAS) tumor antigen. In certain embodiments, the KRAS tumor antigen comprises one or more mutations. In certain embodiments, the peptide comprising at least one heteroclitic neoepitope is displayed on the surface of an antigen presenting cell (APC) via the HLA antigen presentation pathway and a tumor specific immune response is induced. In certain embodiments, the peptide is immunogenic specifically for the subject's HLA type.

In another aspect, an expression vector encodes one or more peptides embodied herein.

In another aspect, an isolated cell comprises an expression vector encodes one or more peptides embodied herein. In certain embodiments, the isolated cell comprises an autologous cell, an allogeneic cell, a haplotype matched cell, a haplotype mismatched cell, a haplo-identical cell, a xenogeneic cell, cell lines or combinations thereof. In certain embodiments, the isolated cell comprises T cells, B cells, natural killer (NK) cells, macrophages, dendritic cells, stem cells, induced pluripotent stem cells (iPSCs) or combinations thereof.

In another aspect, a method of treating cancer in a subject diagnosed with cancer comprises isolating cells from a biological sample subject; culturing the isolated cells with one or more peptides generated by the methods embodied herein, isolating T cells, NK cells, and/or antigen presenting cells cultured with the one or more peptides and expanding the T cells, NK cells, and/or antigen presenting cells to produce a therapeutically effective composition of tumor antigen specific T cells, NK cells, and/or antigen presenting cells; adoptively transferring the tumor antigen specific T cells, NK cells, and/or antigen presenting cells into the subject, thereby treating the subject diagnosed with cancer. In certain embodiments, the peptides comprise one or more heteroclitic neoepitopes.

In another aspect, one or more immunogenic peptides comprise at least a 75% sequence identity to any one or more of SEQ ID NOs: 1-838. In certain embodiments, the immunogenic peptides comprise any one or more of SEQ ID NOs: 1-838. In certain embodiments, the immunogenic peptides comprises an amino acid sequence having at least 95% sequence identity to any one or more of SEQ ID NOs: 1-838 having one or more conservative substitutions. In some embodiments, the immunogenic peptides comprise the amino acid sequence of SEQ ID NO: 2, SEQ ID NO: 3, SEQ ID NO: 833, or SEQ ID NO: 834. In some embodiments, an immunogenic peptide is selected based on the assessment of one or more of contacts between the peptide and HLA cleft residues, solvent-assessable surface area (SASA), peptide rigidity as a measure of CaRMSD of the top 10 predicted structural models for each peptide-HLA complex, surface hydrophobicity, electrostatic potential and/or induction of the greatest T cell expansion and activation as compared to parental peptides.

### DEFINITIONS

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. Definitions of common terms can be found in Singleton et al., Dictionary of Microbiology and Molecular Biology 3rd ed., J. Wiley & Sons New York, NY (2001); March, Advanced Organic Chemistry Reactions, Mechanisms and Structure 5th ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeek, David H Margulies, John Wiley and Sons, Inc., (2003); each of which provide one skilled in the art with a general guide to many of the terms used in the present application.

Standard nomenclature is used for the natural amino acids and their abbreviations. For example, L-alanine is represented with the three-letter abbreviation Ala, or one-letter abbreviation “A”. Where indicated, the “D” stereoisomer of alanine is represented as D-Ala.

Standard nomenclature is used for the bases of DNA, with cytosine, guanosine, adenine, and thymine indicated as “C”, “G”, “A”, and “T”, and codons that encode DNA follow the standard genetic code, for example the amino acid Leu is encoded by TTA, TTG, CTT, CTC, CTA or CTG, and Asp is encoded by GAT or GAC.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude, within 5-fold, and also within 2-fold, of a value. Where particular values are described in the application and claims, unless otherwise stated the term “about” meaning within an acceptable error range for the particular value should be assumed. All numeric values are herein assumed to be modified by the term “about”, whether or not explicitly indicated. The recitation of numerical ranges by endpoints includes all numbers within that range (e.g., 1 to 5 includes 1, 1.5, 2, 2.75, 3, 3.80, 4, and 5).

As used herein, an “adjuvant” refers to a substance that enhances the body's immune response to an antigen or a vaccine and may be added to the formulation that includes the immunizing agent. Adjuvants provide enhanced immune response even after administration of only a single dose of the vaccine. Adjuvants may include, for example, aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or an acrylic acid polyol cross-linked polymer. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers comprise nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the PLURONIC™ brand products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). In a preferred embodiment the adjuvant is at a concentration of about 0.01 to about 50%, at a concentration of about 2% to 30%, at a concentration of about 5% to about 25%, at a concentration of about 7% to about 22%, and at a concentration of about 10% to about 20% by volume of the final product. “Adjuvanted” refers to a composition that incorporates or is combined with an adjuvant.

In the description and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C”, “one or more of A, B, and C” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The term “HLA-peptide structure” as used herein refers to a complex where a peptide is bound to one or a plurality of HLA-haplotypes. The term “amino acid” as used herein refers to naturally occurring and synthetic α, β, γ, and δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. The amino acids can be non-naturally occurring amino acids. Examples of non-naturally occurring amino acids include, but are not limited to, D-amino acids (i.e. an amino acid of an opposite chirality to the naturally-occurring form), N-a-methyl amino acids, C-a-methyl amino acids, b-methyl amino acids and D- or L-b-amino acids. Other non-naturally occurring amino acids include, for example, b-alanine (b-Ala), norleucine (Nle), norvaline (Nva), homoarginine (Har), 4-aminobutyric acid (g-Abu), 2-aminoisobutyric acid (Aib), 6-aminohexanoic acid (e-Ahx), ornithine (orn), sarcosine, a-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

By “cancer” as used herein is meant, a disease, condition, trait, genotype or phenotype characterized by unregulated cell growth or replication as is known in the art; including colorectal cancer, as well as, for example, leukemias, e.g., acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, Primary CNS Lymphoma, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype.

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart. Treatment of a subject also includes a variety of combination therapies with both physical, e.g. surgery, and radiation based treatments.

As used herein, the transitional term “comprising,” which is synonymous with “including,” “containing,” or “characterized by,” is inclusive or open-ended and does not exclude additional, unrecited elements or method steps. When used herein the term “comprising” can be substituted with the term “containing” or “including” or sometimes when used herein with the term “having.” By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “disease” is a state of health of an animal wherein the animal cannot maintain homeostasis, and wherein if the disease is not ameliorated then the animal's health continues to deteriorate.

A “heteroclitic epitope” or “heteroclitic analog” or “heteroclitic neoepitope” or “altered peptide ligand (APL)” are used interchangeably herein, and refers to an altered version of an endogenous peptide sequence (i.e., an analog) engineered to elicit potent immune reactions. Heteroclitic epitopes have increased stimulatory capacity or potency for a specific T cell, as measured by increased responses to a given dose, or by a requirement of lesser amounts to achieve the same response and therefore provide benefit as vaccine components since these epitopes induce T cell responses stronger than those induced by the native epitope. In some embodiments, the potent immune reactions improved HLA binding. In some embodiments, the potent immune reactions comprises improved T cell recognition. In some embodiments, the heteroclitic neoepitopes are derived from tumor antigens or cancer neoantigen.

An “immunogenic peptide” or “antigenic peptide” are used interchangeably herein, and refers to a peptide or epitope that can be recognized by the immune system and elicit an immune response. Immunogenic peptides or antigenic peptides may comprise a motif such that the peptide will bind an MHC molecule and induce a T cell response, or can be recognized by the B cell receptor on the B cell to induce antibody production.

An “immunogenic epitope” or “antigenic epitope” are used interchangeably herein, and refers to a part of an antigen is recognized by the immune system, e.g., by antibodies, B cells, or T cells. In some embodiments, the epitope is the specific piece of the antigen to which an antibody binds. Although epitopes are usually non-self proteins, sequences derived from the host can, in some instances, be recognized.

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4⁺ T cells or CD8⁺ T cells). In some aspects, an immune cell that can be targeted with a targeting moiety (e.g., anti-CD3) comprises naive CD4⁺ T cell. In some aspects, an immune cell comprises a memory CD4⁺ T cell. In some aspects, an immune cell comprises an effector CD4⁺ T cell. In some aspects, an immune cell comprises a naïve CD8⁺ T cell. In some aspects, an immune cell comprises a memory CD8⁺ T cell. In some aspects, an immune cell comprises an effector CD8⁺ T cell. In some aspects, an immune cell comprises a gamma delta T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof.

A “lentivirus” as used herein refers to a genus of the Retroviridae family. Lentiviruses are unique among the retroviruses in being able to infect non-dividing cells; they can deliver a significant amount of genetic information into the DNA of the host cell and are one of the most efficient methods of a gene delivery vector. HIV, SIV, and FIV are all examples of lentiviruses.

“Parenteral” administration of an immunogenic composition includes, e.g., subcutaneous (s.c.), intravenous (i.v.), intramuscular (i.m.), intravitreal (i.v.i.), intra-cisterna magna (i.e.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

By “proteins or molecules of the major histocompatibility complex (MHC)”, “MHC molecules”, “MHC proteins” or “HLLA proteins” is thus meant proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. In some embodiments, MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. In some embodiments, the peptide bound by the MHC molecules of class I originates from an endogenous protein antigen. In some embodiments, the heavy chain of the MHC molecules of class I is preferably an HLA-A, HLA-B or HLA-C monomer, and the light chain is β-2-microglobulin (02M).

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

The terms “sample,” “patient sample,” “biological sample,” and the like, encompass a variety of sample types obtained from a patient, individual, or subject and can be used in a diagnostic, prognostic and/or monitoring assay. The patient sample may be obtained from a healthy subject, a diseased patient, or a patient with lung cancer. In certain embodiments, a sample that is “provided” can be obtained by the person (or machine) conducting the assay, or it can have been obtained by another, and transferred to the person (or machine) carrying out the assay. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiment, a sample comprises cerebrospinal fluid. In a specific embodiment, a sample comprises a blood sample. In another embodiment, a sample comprises a plasma sample. In yet another embodiment, a serum sample is used. The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

The terms “treat,” “treated,” “treating,” “treatment,” and the like are meant to refer to reducing or ameliorating a disorder and/or symptoms associated therewith (e.g., a neoplasia or tumor). “Treating” may refer to administration of the therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED₅₀) of the pharmaceutical composition required. For example, the physician or veterinarian could start doses of the compounds of the disclosure employed in a pharmaceutical composition at levels lower than that required in order to achieve the desired therapeutic effect and gradually increase the dosage until the desired effect is achieved.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

As used herein, an “unnatural amino acid,” “non-natural amino acid”, “modified amino acid” or “chemically modified amino acid” refers to any amino acid, modified amino acid, or amino acid analogue other than the twenty genetically encoded alpha-amino acids. Unnatural amino acids have side chain groups that distinguish them from the natural amino acids, although unnatural amino acids can be naturally occurring compounds other than the twenty proteinogenic alpha-amino acids. In addition to side chain groups that distinguish them from the natural amino acids, unnatural amino acids may have an extended backbone such as beta-amino acids.

Non-limiting examples of non-natural amino acids include selenocysteine, pyrrolysine, homocysteine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof, an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline. In an embodiment of the helicases described herein, one or more amino acids of the helicase are substituted with one or more unnatural amino acids and/or one or more natural amino acids.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

As used herein, the term “virus” includes any type of virus or virus vector. For example, adenovirus, adeno-associated virus (AAV), recombinant adeno-associated virus (rAAV), herpes simplex virus, lentivirus, retrovirus, alphavirus, flavivirus, rhabdovirus, measles virus, Newcastle disease virus, poxvirus, vaccinia virus, modified Ankara virus, vesicular stomatitis virus, picornavirus. In various embodiments the virus is a chimeric virus, a synthetic virus, a recombinant virus, a mosaic virus or a pseudotyped virus. In some embodiments an AAV/rAAV disclosed herein can AAV1, AAV2, AAV3, AAV4, AAV5, AAV6, AAV7, AAV8, AAV9, and/or AAV10 capsid.

The vaccine of the present disclosure may ameliorate a disease as described herein. By “ameliorate” is meant decrease, suppress, attenuate, diminish, arrest, or stabilize the development or progression of a disease (e.g., a neoplasia, tumor, autoimmunity, infection, etc.).

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In preferred embodiments, the genes, nucleic acid sequences, amino acid sequences, peptides, polypeptides and proteins are human. The term “gene” is also intended to include variants.

The practice of the present disclosure employs, unless otherwise indicated, techniques of chemistry, molecular biology, microbiology, recombinant DNA, genetics, immunology, cell biology, cell culture and transgenic biology. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Any compositions or methods provided herein can be combined with one or more of any of the other compositions and methods provided herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a series of Word plots of 9-mer peptide binding consensus sequences from each HLA allele. Data was accessed through HLAthena browser.

FIG. 2 shows the NetMHC predicted binding affinity for parental KRAS G12D epitope (boxed) and all altered peptide ligand heteroclitic epitopes across the panel of HLAs. Gray asterisks indicate weak binding epitopes, Black asterisks indicate strong binders. Figure discloses SEQ ID NOS 639-640, 840-848, 707, 710, 849, 642, 639, 846, 850, 640, 642, 851-866, 639, 846, 642, 640-641 and 867-875, respectively, in order of columns.

FIG. 3 is a series of schematic representations showing the structural modelling of the KRAS G12D epitope GADGVGKSA (SEQ ID NO: 639) on HLA-A*02:01. Rotamer substitution of anchor amino acid residues 9 from alanine to leucine, 2 from alanine to leucine, and 6 from glycine to leucine. Contacts are shown. HLA contact residues are shown. Black arrows indicate interactions with increased contacts. Figure discloses SEQ ID NOS 639-640 and 842-843, respectively, in order of appearance.

FIG. 4A, FIG. 4B show a heatmap and a table and demonstrating the HLA-specific Mutant KRAS minimal epitope targets validated by mass-spec for heteroclitic peptide design.

FIG. 4B shows a compiled list of mutant KRAS minimal epitopes and HLA restriction that have been confirmed by mass spectrometry. FIG. 4B discloses SEQ ID NOS 4, 13, 163, 1, 169, 180, 186, 1, 214, 203, 163, 169, 180, 186, 203, 163, 169, 180, 186, 1, 203, 169, 180, 567, 574, 602, 639, 567, 595, 652, 691, 567, 595 and 652, respectively, in order of appearance.

FIG. 5A-FIG. 5C show a table, a heatmap and a plot demonstrating the amino acid modifications of mKRAS epitopes predicted to improve HLA binding affinity for HLA-A*02:01. FIG. 5A shows peptide binding consensus sequence for 10-mer (left) and 9-mer (right) epitopes for HLA-A*02:01. Amino acids are shaded by physiochemical property. FIG. 5B shows heat map of predicted binding affinity (nM) of parental G12V 10-mer and 9-mer and APLs to HLA-A*02:01. FIG. 5C shows a compiled list of mutant 10-mer and 9-mer KRAS minimal epitopes restricted to HLA-A*02:01 (parental) and modified altered peptide ligands (APL). Figure discloses SEQ ID NOS 4, 4, 6-21, 4, 4 and 6-21, respectively, in order of columns.

FIG. 6A-FIG. 6C show a table, a heatmap and a plot demonstrating the amino acid modifications of mKRAS epitopes predicted to improve HLA binding affinity for HLA-A*03:01. FIG. 6A shows peptide binding consensus sequence for 10-mer (left) and 9-mer (right) epitopes for HLA-A*03:01. Amino acids are shaded by physiochemical property. FIG. 6B shows heat map of predicted binding affinity (nM) of parental 10-mer (left—KRAS G12D, G12V, G12C) and 9-mer (right—KRAS G12R, G12V) and APLs to HLA-A*03:01. X indicates location of KRAS mutation. FIG. 6C shows a compiled list of mutant 10-mer and 9-mer KRAS minimal epitopes restricted to HLA-A*03:01 (parental) and modified altered peptide ligands (APL) X indicates location of KRAS mutation. Figure discloses SEQ ID NOS 876-883, 877, 884-891, 876-883, 877 and 884-891, respectively, in order of columns.

FIG. 7A-FIG. 7C show a table, a heatmap and a plot demonstrating the amino acid modifications of mKRAS epitopes predicted to improve HLA binding affinity for HLA-A*11:01. FIG. 7A shows peptide binding consensus sequence for 10-mer (left) and 9-mer (right) epitopes for HLA-A*11:01. Amino acids are shaded by physiochemical property. FIG. 7B shows heat map of predicted binding affinity (nM) of parental 10-mer (left—KRAS G12D, G12R, G12V, G12C) and 9-mer (right—KRAS G12D, G12R, G12V) and APLs to HLA-A*11:01. X indicates location of KRAS mutation. FIG. 7C shows a compiled list of mutant 10-mer and 9-mer KRAS minimal epitopes restricted to HLA-A*11:01 (parental) and modified altered peptide ligands (APL). X indicates location of KRAS mutation. Figure discloses SEQ ID NOS 892-903, 900, 904-906, 892-903, 900 and 904-906, respectively, in order of columns.

FIG. 8A-FIG. 8C show a table, a heatmap and a plot demonstrating the amino acid modifications of mKRAS epitopes predicted to improve HLA binding affinity for HLA-A*30:01. FIG. 8A shows peptide binding consensus sequence for 10-mer (left) F and 9-mer (right) epitopes for HLA-A*30:01. Amino acids are shaded by physiochemical property. FIG. 8B shows heat map of predicted binding affinity (nM) of parental 10-mer (left—KRAS G12R, G12V) and 9-mer (right-KRAS G12R, G12V) and APLs to HLA-A*30:01. X indicates location of KRAS mutation. FIG. 8C shows a compiled list of mutant 10-mer and 9-mer KRAS minimal epitopes restricted to HLA-A*30:01 (parental) and modified altered peptide ligands (APL). X indicates location of KRAS mutation. Figure discloses SEQ ID NOS 907-913, 886, 914-919, 907-913, 886 and 914-919, respectively, in order of columns.

FIG. 9A-FIG. 9C show a table, a heatmap and a plot demonstrating the amino acid modifications of mKRAS epitopes predicted to improve HLA binding affinity for HLA-A*68:01. FIG. 9A shows peptide binding consensus sequence for 10-mer (left) and 9-mer (right) epitopes for HLA-A*68:01. Amino acids are shaded by physiochemical property. FIG. 9B shows heat map of predicted binding affinity (nM) of parental 10-mer (left—KRAS G12D G12C, G12R, G12V) and 9-mer (right—KRAS G12V) and APLs to HLA-A*68:01. X indicates location of KRAS mutation. FIG. 9C shows a compiled list of mutant 10-mer and 9-mer KRAS minimal epitopes restricted to HLA-A*68:01 (parental) and modified altered peptide ligands (APL). X indicates location of KRAS mutation. Figure discloses SEQ ID NOS 892, 920, 894, 921-923, 180, 279, 515-516, 892, 920, 894, 921-923, 180, 279, 515-517 and 181, respectively, in order of columns.

FIG. 10A-FIG. 10C show a table, a heatmap and a plot demonstrating the amino acid modifications of mKRAS epitopes predicted to improve HLA binding affinity for HLA-B*07:02. FIG. 10A shows peptide binding consensus sequence for 10-mer epitopes for HLA-B*07:02. Amino acids are colored by physiochemical property. FIG. 10B shows heat map of predicted binding affinity (nM) of parental 10-mer KRAS G12D and G12R epitopes and APLs to HLA-B*07:02. X indicates location of KRAS mutation. FIG. 10C shows a compiled list of mutant 10-mer KRAS minimal epitopes restricted to HLA-B*07:02 (parental) and modified altered peptide ligands (APL). X indicates location of KRAS mutation. Figure discloses SEQ ID NOS 924-930 and 924-930, respectively, in order of appearance.

FIG. 11A-FIG. 11C show a table, a heatmap and a plot showing the amino acid modifications of mKRAS epitopes predicted to improve HLA binding affinity for HLA-C*01:02. FIG. 11A shows peptide binding consensus sequence for 9-mer epitopes for HLA-C*01:02. Amino acids are colored by physiochemical property. FIG. 11B shows heat map of predicted binding affinity (nM) of parental 9-mer KRAS G12V epitopes and APLs to HLA-C*01:02. FIG. 11C shows a compiled list of known mutant 10-mer KRAS minimal epitopes restricted to HLA-C*01:02 (parental) and modified altered peptide ligands (APL). Figure discloses SEQ ID NOS 602-605, 607 and 602-607, respectively, in order of appearance.

FIG. 12A-FIG. 12C show a table, a heatmap and a plot showing the amino acid modifications of mKRAS epitopes predicted to improve HLA binding affinity for HLA-C*08:02. FIG. 12A shows peptide binding consensus sequence for 10-mer (left) and 9-mer (right) epitopes for HLA-C*08:02. Amino acids are colored by physiochemical property. FIG. 12B shows heat map of predicted binding affinity (nM) of parental 10-mer (left—KRAS G12D) and 9-mer (right-KRAS G12D) and APLs to HLA-C*08:02. FIG. 12C shows a compiled list of mutant 10-mer and 9-mer KRAS minimal epitopes restricted to HLA-C*08:02 (parental) and modified altered peptide ligands (APL). Figure discloses SEQ ID NOS 567, 931, 634-635, 637-644, 567, 567 and 634-644, respectively, in order of columns.

FIG. 13A-FIG. 13C show a table, a heatmap and a plot demonstrating the amino acid modifications of mKRAS epitopes predicted to improve HLA binding affinity for HLA-C*03:03. FIG. 13A shows peptide binding consensus sequence for 10-mer (left) and 9-mer (right) epitopes for HLA-C*03:03. Amino acids are colored by physiochemical property. FIG. 13B shows heat map of predicted binding affinity (nM) of parental 10-mer (left—KRAS G12V) and 9-mer (right-KRAS G12V) and APLs to HLA-C*03:03. FIG. 13C shows a compiled list of mutant 10-mer and 9-mer KRAS minimal epitopes restricted to HLA-C*03:03 (parental) and modified altered peptide ligands (APL). Figure discloses SEQ ID NOS 595, 773-775, 601, 652, 779-781, 653, 783, 595, 773-775, 601, 647, 652, 779-781, 653 and 783, respectively, in order of columns.

FIG. 14A-FIG. 14C show a series of heatmaps, a schematic, and a graph showing a KRAS APL binding assay-MBL tetramer quick switch assay. FIG. 14A shows KRAS altered peptide ligand binding assay to HLA-A*11:01 tetramers—overview of tetramer binding assay. HLA-A*11:01 tetramers were incubated with 10 mM KRAS parental epitope or APL, 4 hr, RT. Peptide loading was then quantified by flow cytometry analysis with anti-exiting peptide antibody.

FIG. 14B shows peptide loading calculated as a percentage relative to (+) control peptide and no peptide (−) control. FIG. 14C shows NetMHC predicted binding affinities for HLA-A*11:01-specific KRAS peptides and APLs tested with this assay. FIG. 14C discloses SEQ ID NOS 892-894, 896, 898-901 and 903-905, respectively, in order of column.

FIG. 15 show a series of graphs showing the results obtained from a KRAS APL HLA-A*11:01 binding assay. Percent binding of KRAS G12D, G12R, G12V, and G12C 10- and 9-mer parental epitopes and altered peptide ligands. Percent binding of each APL is calculated relative to the respective parental epitope. Figure discloses SEQ ID NOS 1-2, 239, 3, 243-244, 214, 246, 248, 250-251, 203, 254, 255, 257, 259-260, 163, 262, 264, 266-267, 169, 270-271, 273, 275-276, 180, 278, 280, 282-283, 186, 286-287, 289 and 291-292, respectively in order of appearance.

FIG. 16A-FIG. 16D show a schematic and a series of graphs demonstrating KRAS G12D altered peptide ligands prime T cell responses against parental antigen in vitro. An in vitro immunogenicity assay was conducted for KRAS G12D altered peptide ligands in an HLA-A*11:01 healthy donor. FIG. 16A shows an overview of in vitro T cell priming and expansion assay. Healthy donor dendritic cells were generated by adherence of monocytes to flasks and differentiated in 800 U/mL hGMCSF and 400 IU/mL hIL-4 for 7 days. Dendritic cells were then matured in hIL-1B, IL-6, TNFα, and PGE2 for 2 days. Mature dendritic cells were then collected and pulsed with 40 μg/mL parental or altered peptide ligand for 2 hrs. Pulsed dendritic cells were washed twice with PBS and then co-cultured with autologous CD8⁺ T cells in the presence of hIL-15, hIL-6, and hIL-21. 7 days later, more peptide-pulsed dendritic cells were added to each co-culture. The following day, fresh hIL2 and IL7 were added. 7 days post the final dendritic cell culture, T cells were collected and stained with an HLA-A*11:01 tetramer containing the parental epitope sequence. FIG. 16B shows flow cytometry gates of HLA-A*11:01 KRAS G12D 10-mer+T cells (gated on HLA-DR-CD45RO+ events). FIG. 16C shows quantified populations of HLA-A*11:01 G12D 10-mer tet⁺ T cells in control, parental epitope, or altered peptide ligand expansions. FIG. 16D shows KRAS G12D-HLA-A*11:01 tetramer staining of ATVGADGVGK (SEQ ID NO: 244) expanded T cells 7 days after 3^{rd} peptide boost. No tetramer control was used as negative control. Figure discloses SEQ ID NOS 1-2, 239, 3, 243-244, 2, 239, 3, 243-244 and 244, respectively, in order of appearance.

FIG. 17 shows a schematic of KRAS G12D parental and altered peptide ligand-HLA contacts and surface structure. Structural modeling of KRAS G12D 10-mer parental epitope (G12D) or altered peptide ligands in HLA-A*11:01. Top row indicates contacts between the 10-mer peptide and HLA binding cleft. Bottom row shows surface area of peptide-HLA complex. Measurements are indicated in FIG. 20. Figure discloses SEQ ID NOS 2, 239, 3 and 243-244, respectively, in order of appearance.

FIG. 18 shows a schematic of KRAS G12D parental and altered peptide ligand-HLA hydrophobicity measurements. Hydrophobicity measurements of KRAS G12D parental and altered peptide ligands-HLA complexes. Surface lipophilicity values are mapped. Figure discloses SEQ ID NOS 2, 239, 3 and 243-244, respectively, in order of appearance.

FIG. 19 shows a schematic of KRAS G12D parental and altered peptide ligand-Electrostatic potential measurements. Electrostatic potential measurements of KRAS G12D parental and altered peptide ligands-HLA complexes are shown. Surface electrostatic potential values are mapped. Figure discloses SEQ ID NOS 2, 239, 3 and 243-244, respectively, in order of appearance.

FIG. 20 shows a table of the structural feature measurements of KRAS G12D 10-mer parental epitope and altered peptide ligands modeled in the context of HLA-A*11:01. Figure discloses SEQ ID NOS 1-2, 239, 3 and 243-244, respectively, in order of appearance.

FIG. 21A-FIG. 21F show a series of plots and schematics demonstrating that engineered peptides display enhanced contact points and solvent accessible surface area, and reduced rigidity in a murine PDAC model. FIG. 21A shows that mice were vaccinated twice 7 days apart with PBS, 50 μg APL, or 5 μg parental peptide. Seven days after the last vaccine dose, splenocytes were restimulated with APL or parental peptides, overnight, and IFNγ production was measured by ELISpot. Two-way ANOVA followed by Tukey's multiple comparisons test was performed.

FIG. 21B shows in silico structural modeling of APL or parental peptide in murine H2-Kb (murine MHC class I molecule H2-Kb, minimal epitope of peptide 44, parental or engineered amino acid residue). Structural features analyzed from parental or APL MHC models. Number of contacts between MHC binding cleft and peptide (FIG. 21C) or mutated residue (FIG. 21D) are shown.

FIG. 21E shows Ca-RMSD measure of peptide rigidity. FIG. 21F shows solvent accessible surface area (SASA). Unpaired students t test was performed. (ns=P>0.05, *P≤0.05, **P≤0.01, ***P≤0.001, ****P≤0.0001).

FIG. 22A-FIG. 22C demonstrate that KRAS G12D APL1-HLA*11:01 binding and immunogenicity of engineered antigens show superior binding and T Cell Responses. FIG. 22A shows non-linear regression curves and calculated EC50s for binding between HLA-A*11:01 and a parental KRAS G12D 10-mer peptide and 7 engineered antigens. FIG. 22B shows that 1×10⁶ expanded CD8 T cells treated with no peptide, irrelevant peptide, parental peptide, or APL were seeded in an ELISpot capture plate coated with anti-human IFNγ antibody. T cells were stimulated with control or parental antigen overnight and IFNγ capture was read out. Two-way ANOVA followed by uncorrected Fischer's LSD test was performed. (ns=P>0.05, *P≤0.05, **P≤0.01, ***P≤0.001, ****P≤0.0001). FIG. 22C shows in vitro immunogenicity testing of HLA-A*11:01 parental epitope and its engineered counterpart (APL1). Human monocyte-derived DCs (moDCs) differentiated from healthy donor PBMCs were pulsed with 100 μM parental or APL1 plus 02-microglobulin (4 hours) at 37 °C. After peptide pulsing, moDCs were washed with PBS and co-cultured with 6×10⁶ autologous CD8 T cells (plus IL-2, IL-7, IL-15, and IL-21) (7 days). Boosts were performed with peptide-pulsed moDCs on day 7, 14, and 21. T cells were collected and stained with HLA-A*11:01 tetramer loaded with parental peptide or were subject to IFNγ ELISpot.

FIG. 23 shows a series of plots showing that KRAS G12D APL4 demonstrates greater T cell responses compared to parental epitope in a different HLA*11:01 human donor. In vitro immunogenicity testing of HLA-A*11:01 parental epitope and its engineered counterpart (APL4). Human monocyte-derived DCs (moDCs) differentiated from healthy donor PBMCs were pulsed with 100 μM parental or APL4 plus 02-microglobulin (4 hours) at 37 °C. After peptide pulsing, moDCs were washed with PBS and co-cultured with 6×10⁶ autologous CD8 T cells (plus IL-2, IL-7, IL-15, and IL-21) (7 days). Boosts were performed with peptide-pulsed moDCs on day 7, 14, and 21. T cells were collected and stained with HLA-A*11:01 tetramer loaded with parental peptide (Left) or subject to IFNγ ELISPOT (Right).

FIG. 24A_shows structural models of A11-KRAS G12D parental epitope (left), All-KRAS G12D APL1, and All-KRAS G12D APL4 in the context of HLA-A*11:01. Top 10 energetically favorable models of the peptide are shown for each peptide (n=10). FIG. 24B CaRMSD was calculated for the 10-mer peptide in each model compared to all other models for that epitope. Two way ANOVA was performed followed by Tukey's multiple comparisons test.

FIG. 24C shows structural analysis of peptide neoantigen residue (G12D) in contact with HLA cleft residues that are associated with stabilization of peptide-HLA interactions (from left to right, Arg 114, Gln155, Gln70, TRP147, Thr73).

FIG. 25A shows binding affinity for KRAS G12D APLs in the context of HLA-A*03:01. FIG. 25B shows binding affinity for KRAS G12D APLs in the context of HLA-B*07:02

### DETAILED DESCRIPTION

This disclosure relates to the utilization of structural features of neoantigen epitope binding to diverse HLA subtypes to predict optimal anchor modifications that will improve HLA binding and epitope immunogenicity in an HLA-specific manor. The result of this prediction is the optimized heteroclitic peptide vaccine candidate for a patient's specific HLA type. This technology is applicable for shared neoantigens such as mutant KRAS G12D which has been shown to be weakly immunogenic in patients in vaccination settings. In certain aspects, the disclosure provides herein KRAS G12D heteroclitic epitope vaccine candidates designed and modelled for diverse HLA subtypes. Additionally, the software described herein is applicable to private patient neoantigen targets where the algorithm can prioritize immunogenic neoantigen targets based on structural features of neoantigen display as well as identify optimal heteroclitic peptides for the patient's HLA subtypes. In some embodiments, a heteroclitic peptide disclosed herein is selected based on the assessment of one or more of contacts between the peptide and HLA cleft residues, solvent-assessable surface area (SASA), peptide rigidity as a measure of CaRMSD of the top 10 predicted structural models for each peptide-HLA complex, surface hydrophobicity, electrostatic potential and/or induction of the greatest T cell expansion and activation as compared to parental peptides.

### Immune System and Antigen Presentation

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells and are then presented to naive or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines. The human leukocyte antigen (HLA) system is a gene complex encoding the major histocompatibility complex (MHC) proteins in humans.

The present disclosure provides methods for predicting peptides capable of binding to HLA alleles. The embodiments comprise a set of candidate peptide sequences and identifying one or more structural features indicative of occupancy of the candidate peptides on the binding pocket or cleft of HLA alleles. This is done, for example, by identifying positional sequence similarity of peptide epitopes that bind to one or a plurality of HLA haplotypes. The HLA-peptide structure having a sequence identity greater than a control HLA-peptide structure is selected and the machine learning algorithm models the heteroclitic neoepitopes into an HLA cleft by introducing amino acid rotamers with the lowest energy confirmation at each position. These structural features are input into a machine learning algorithm model to simulating occupancy of one or more binding peptides and one or more non-binding peptides on the HLA binding pocket in a crystal structure of the HLA allele or the crystal structure of a similar HLA allele. The structural features can be extracted from the output models generated during the simulations. The structural features can also be identified using a machine learning algorithm model that infers occupancy of the one or more candidate peptides on the HLA binding pocket. The inference can be based on the model being trained with simulated models of peptides verified to bind to the HLA allele and verified peptides that do not bind to the HLA allele.

Vaccine therapies (e.g., cancer and infections) rely on accurate selection of immunizing peptides to potentiate immune responses (e.g., against tumor-specific neoepitopes or viral epitopes). Given the patient's particular complement of HLA alleles, the ability to predict which epitopes will be presented is a fundamental prerequisite for successful vaccine design. Additionally, given the unique accumulation of mutations in different tumors as well as the patient's particular complement of HLA alleles, the ability to predict which epitopes will be presented is a fundamental prerequisite for successful cancer vaccine design.

In certain embodiments, an initial input of a candidate peptide or a set of candidate peptides, e.g. KRAS peptides is provided. In certain embodiments, the candidate peptide or set of candidate peptides may be obtained from a subject or group of subjects in need of an immune response or modified immune response. In certain embodiments, candidate peptides can be identified in a peptide sequence database (e.g., derived from sequencing of subjects having a specific condition where an immunogenic composition would be useful).

In certain embodiments, a peptide sequence database includes HLA allele binding and non-binding peptides. Candidate peptides can be isolated and sequenced for each HLA-allele to identify HLA-binding peptides. In particular embodiments, candidate peptides can be obtained by providing a) a population of cells which expresses a single class I HLA allele or a single pair of class II HLA alleles (one α-chain and one β-chain); b) isolating the respective HLA-peptide complexes from said cells; c) isolating peptides from said HLA-peptide complexes; and d) sequencing the peptides.

The population of cells may express either a single class I HLA allele, a single pair of class II HLA alleles, or a single class I HLA allele and a single pair of class II HLA alleles. Suitable cell populations include, e.g., class I deficient cells lines in which a single HLA class I allele is expressed, class II deficient cell lines in which a single pair of HLA class II alleles are expressed, or class I and class II deficient cell lines in which a single HLA class I and/or single pair of class II alleles are expressed.

The population of cells may be professional antigen presenting cells such as macrophages, B cells and dendritic cells. In certain embodiments, the cells are B cells or dendritic cells. In certain embodiments, the cells are tumor cells or cells from a tumor cell line. In particular embodiments, the cells are cells isolated from a patient.

In some embodiments, the population of cells are further modified, such as by increasing or decreasing the expression and/or activity of at least one gene. In certain embodiments, the gene encodes a member of the immunoproteasome. In some embodiments, the immunoproteasome is involved in the processing of HLA class I binding peptides and includes the LMP2 (βii), MECL-1 (β2i), and LMP7 (β5i) subunits. In some embodiments, the immunoproteasome can also be induced by interferon-gamma. Accordingly, in some embodiments, the population of cells may be contacted with one or more cytokines, growth factors, or other proteins. In some embodiments, the cells are stimulated with inflammatory cytokines such as interferon-gamma, IL-Iβ, IL-6, and/or TNF-α. The population of cells may also be subjected to various environmental conditions, such as stress (heat stress, oxygen deprivation, glucose starvation, DNA damaging agents, etc.). In some embodiments, the cells are contacted with one or more of a chemotherapy drug, radiation, targeted therapies or immunotherapy. The methods disclosed herein can therefore be used to study the effect of various genes or conditions on HLA peptide processing and presentation. In particular embodiments, the conditions used are selected so as to match the condition of the patient for which the population of HLA-peptides is to be identified.

In some embodiments, Any HLA allele may be expressed in the cell population. In some embodiments, it will be of interest to sequentially perform the methods provided herein for different HLA alleles, such that resulting datasets can be used in combination. In certain embodiments, the HLA allele is selected so as to correspond to a genotype of interest. In a certain embodiment, the HLA allele is a mutated HLA allele, which may be non-naturally occurring allele or a naturally occurring allele in an afflicted patient. In some embodiments, the methods disclosed herein have the further advantage of identifying HLA binding peptides for HLA alleles associated with various disorders as well as alleles which are present at low frequency. Accordingly, in one method the HLA allele is present at a frequency of less than 10%, 9%, 8%, 7%, 6%, 5%, 4%, 3%, 2%, 1% within a population. In some embodiments, the HLA allele is present at a frequency of less than 1% within a population.

In some embodiments, the methods further comprise isolating peptides from HLA-peptide complexes and sequencing the peptides to identify input candidate peptides. In some embodiments, the peptides are isolated from the complex by any method known to one of skill in the art, such as acid elution. In some embodiments, any suitable sequencing method may be used, including methods employing mass spectrometry, such as liquid chromatography-mass spectrometry (LC-MS or LC-MS/MS, or alternatively HPLC-MS or HPLC-MS/MS).

In some embodiments, an HLA-allele specific binding peptide sequence database comprises at least 1000 different binding peptide sequences. The methods disclosed herein may also be used to generate a database comprising the HLA-allele specific binding peptide sequences for more than one HLA-allele, for at least two different HLA-alleles, for at least five, for at least ten, fifteen, twenty, thirty or more different alleles.

In one aspect, the present disclosure provides a plurality of HLA-allele specific binding peptides, or the sequences thereof, which peptides correspond to the peptides which are presented by one specific HLA allele. More particularly, in some embodiments, an HLA-allele specific binding peptide sequence database provided is obtained by carrying out the method described herein.

### Structural Features of Peptides

In one aspect, the present disclosure provides methods for identifying HLA-allele specific binding peptides, which method comprises analyzing structural features indicative of occupancy of peptides on the binding pocket of HLA alleles. In certain embodiments, the structural features are amino acid residues capable of fitting a model of peptide occupancy on the binding pocket of HLA alleles (e.g., enrichment in hydrophobicity, exposed hydrophobic surface and charges determined by the peptide's conformation within the binding pocket, as well as the size and position of the various amino acid side chains). In certain embodiments, the structural features are energetic features that are encoded not by peptide sequence, but by modeled three-dimensional structures of peptide occupancy on the binding pocket of an HLA allele (e.g., energies of attraction, repulsion, and solvation; energies of side chain and backbone hydrogen bonds; and energies and probabilities of side chain and backbone conformations) (see, e.g., Alford R F, Leaver-Fay A, Jeliazkov J R, O'Meara M J, DiMaio F P, Park H, et al. The Rosetta all-atom energy function for macromolecular modeling and design. J Chem Theory Comput. (2017) 13:3031-48; and Riley et al., Structure Based Prediction of Neoantigen Immunogenicity. Front Immunol. 2019 Aug. 28; 10:2047).

In certain embodiments, the binding pocket is determined by a structural analysis. Non-limiting structural analysis methods include X-ray crystallography and nuclear magnetic resonance (NMR) spectroscopy (Alberts B, Johnson A, Lewis J, et al. Molecular Biology of the Cell. 4th edition. New York: Garland Science; 2002. Analyzing Protein Structure and Function). In certain embodiments, a crystal structure is used to determine the binding pocket. Any HLA-allele crystal structure may be used to simulate occupancy of peptides in the binding pocket. In certain embodiments, a structure is obtained by generating a crystal structure of an HLA-allele. In certain embodiments, the crystal structures for HLA molecules are obtained from a database (e.g., the Protein Data Bank (PDB, resb.org)). In certain embodiments, structures of similar HLA alleles are used if a structure for the HLA-allele of interest is unavailable or cannot be generated. In certain embodiments, similar HLA-alleles include HLA alleles having the highest similarity between the amino acid sequences of the binding pocket. In some embodiments, the similarity of the binding pocket can be computed as the sum of pair-wise residue similarities according to a 20×20 amino acid similarity matrix. In certain embodiments, similar HLA-alleles include HLA alleles having similarity between the binding motifs (i.e. two alleles are considered similar if they bind similar peptides). In certain embodiments, the HLA alleles bind to two or more of the same peptides. In certain embodiments, similar HLA-alleles include HLA alleles from the same class (e.g., HLA-A, HLA-B, HLA-C, HLA-E, HLA-F, HLA-G, HLA-K and HLA-L). In certain embodiments, similar HLA-alleles include HLA alleles having the highest amino acid sequence identity to the HLA-allele of interest. In certain embodiments, similar HLA-alleles include HLA alleles having at least 85%, 90% 95%, 96%, 97%, 98%, or 99% amino acid sequence identity to the HLA-allele of interest.

In certain embodiments, structural features described herein are identified by simulating models of how the candidate peptides occupy the HLA binding pocket. In certain embodiments, the method comprises analyzing simulations of the occupancy of peptides on the binding pocket of HLA alleles.

In certain embodiments, how peptides occupy the HLA binding pocket is computationally simulated. For example, using a Multiple Sequence Comparison by Log-Expectation (MUSCLE) peptide sequence alignment, which takes into consideration positional sequence similarity, of the candidate peptide epitopes, e.g. KRAS peptides, with all 8-8-mer, 9-mer, 11-mer, 12-mer, 13-mer, 14-mer, 15-mer, 16-mer, 17-mer, 18-mer, 19-mer, 20-mer, 21-mer, 22-mer, 23-mer, 24-mer, or 25-mer epitopes that bind each HLA and have a crystal structure available.

In certain embodiments, the immunogenic peptides comprise from about five amino acids to about 50 amino acids. In certain embodiments, the immunogenic peptides comprise from about seven amino acids to about 45 amino acids. In certain embodiments, the immunogenic peptides comprise from about eight amino acids to about 40 amino acids. In certain embodiments, the immunogenic peptides comprise from about nine amino acids to about 35 amino acids. In certain embodiments, the immunogenic peptides comprise from about nine amino acids to about 30 amino acids. In certain embodiments, the immunogenic peptides comprise from about nine amino acids to about 25 amino acids.

In certain embodiments, the immunogenic peptides comprise from about five amino acids to about 50 amino acids include an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-838. In some embodiments, an immunogenic peptide disclosed herein comprises the amino acid sequence of any one of SEQ ID NOs: 1-838. In some embodiments, an immunogenic peptide disclosed herein comprises the amino acid sequence of any one of SEQ ID NOs: 1-838 having one or more conserved amino acid substitutions. In certain embodiments, the immunogenic peptides comprise an amino acid sequence having at least 95% sequence identity to any one or more of SEQ ID NOs: 1-838 having one or more conservative substitutions. In certain embodiments, the immunogenic peptide comprise SEQ ID NO: 2. In some embodiments, the immunogenic peptide comprises SEQ ID NO: 3. In some embodiments, the immunogenic peptide comprises SEQ ID NO. 833. In some embodiments, the immunogenic peptide comprises SEQ ID NO. 834

In certain embodiments, the immunogenic peptides comprise five consecutive amino acids of any one or more of SEQ ID NOs: 1-838. In certain embodiments, the immunogenic peptides comprise five consecutive amino acids of any one or more of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. In certain embodiments, the immunogenic peptides comprise six consecutive amino acids of any one or more of SEQ ID NOs: 1-838. In certain embodiments, the immunogenic peptides comprise six consecutive amino acids of any one or more of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. In certain embodiments, the immunogenic peptides comprise seven consecutive amino acids of any one or more of SEQ ID NOs: 1-838. In certain embodiments, the immunogenic peptides comprise seven consecutive amino acids of any one or more of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. In certain embodiments, the immunogenic peptides comprise eight consecutive amino acids of any one or more of SEQ ID NOs: 1-838. In certain embodiments, the immunogenic peptides comprise eight consecutive amino acids of any one or more of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. In certain embodiments, the immunogenic peptides comprise nine consecutive amino acids of any one or more of SEQ ID NOs: 1-838. In certain embodiments, the immunogenic peptides comprise nine consecutive amino acids of any one or more of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834.

In certain embodiments, the immunogenic peptides comprise from about seven amino acids to about 45 amino acids include an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-838. In certain embodiments, the immunogenic peptides comprise from about eight amino acids to about 40 amino acids.

In certain embodiments, the immunogenic peptides comprise from about nine amino acids to about 35 amino acids include an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-838.

In certain embodiments, the immunogenic peptides comprise from about nine amino acids to about 30 amino acids include an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-838.

In certain embodiments, the immunogenic peptides comprise from about nine amino acids to about 25 amino acids include an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-838.

In certain embodiments, mutant KRAS epitopes comprise G12V, G12D, G12C, G12R, G12A, G13D or combinations thereof.

In certain embodiments, KRAS variants of the present disclosure comprise an amino acid sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 1-838. In some embodiments, a KRAS variant disclosed herein comprises the amino acid sequence of any one of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. In certain embodiments, the KRAS variant comprise an amino acid sequence having at least 95% sequence identity to any one or more of SEQ ID NOs:. In some embodiments, a KRAS variant disclosed herein comprises the amino acid sequence of any one of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834. having one or more conservative substitutions. In certain embodiments, the KRAS variant comprise SEQ ID NO: 2. In some embodiments, the KRAS variant comprises SEQ ID NO: 3. In some embodiments, the KRAS variant comprises SEQ ID NO. 833. In some embodiments, the KRAS variant comprises SEQ ID NO. 834.

In certain embodiments, the KRAS and/or mKRAS peptides comprise one or more modified amino acids, unnatural amino acids, substituted amino acids or combinations thereof. Accordingly, the KRAS and/or mKRAS peptides comprising any one of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834 further comprise one or more modified amino acids, unnatural amino acids, substituted amino acids or combinations thereof. Non-limiting examples of non-natural amino acids include selenocysteine, pyrrolysine, homocysteine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof, a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline.

### Candidate Therapeutic Peptides

In certain embodiments, the methods described herein are used to determine an effective neoantigen vaccine. In this context, it is of interest to determine which neoantigen peptides are likely to bind to a subject's HLA so as to effectively function as immunogenic peptides.

In some embodiments, subject specific HLA alleles or HLA genotype of a subject may be determined by any method known in the art.

One of the barriers to developing curative and tumor-specific immunotherapy is the identification and selection of highly specific and restricted tumor antigens to avoid autoimmunity. Tumor neoantigens, which arise as a result of genetic change (e.g., inversions, translocations, deletions, missense mutations, splice site mutations, etc.) within malignant cells, represent the most tumor-specific class of antigens. Neoantigens have rarely been used in cancer vaccine or immunogenic compositions due to technical difficulties in identifying them, selecting optimized neoantigens, and producing neoantigens for use in a vaccine or immunogenic composition. These problems may be addressed by: identifying mutations in neoplasias/tumors which are present at the DNA level in tumor but not in matched germline samples from a high proportion of subjects having cancer; analyzing the identified mutations by the methods embodied herein to generate a plurality of neoantigen epitopes that are expressed within the neoplasia/tumor and that bind to a high proportion of patient HLA alleles; and synthesizing the plurality of the neoantigenic peptides for use in a cancer vaccine or immunogenic composition suitable for treating a high proportion of subjects having cancer.

In some embodiments, a therapeutic vaccine disclosed herein may include: (1) identification of mutated peptides that can bind to HLA molecules of a high proportion of individuals and (2) formulating the drug as a multi-epitope vaccine of long peptides. In some embodiments, targeting as many mutated epitopes as possible can take advantage of the enormous capacity of the immune system, prevents the opportunity for immunological escape by down-modulation of a particular immune targeted gene product, and compensates for the known inaccuracy of epitope prediction approaches. In some embodiments, a synthetic peptide described herein provide a particularly useful means to prepare multiple immunogens efficiently and to rapidly translate identification of mutant epitopes to an effective vaccine. In some embodiments, peptides can be readily synthesized chemically and easily purified utilizing reagents free of contaminating bacteria or animal substances. The small size allows a clear focus on the mutated region of the protein and also reduces irrelevant antigenic competition from other components (unmutated protein or viral vector antigens). In some embodiments, a vaccine disclosed herein can be used in (3) combination with a strong vaccine adjuvant. In some embodiments, an effective vaccine can require a strong adjuvant to initiate an immune response. In some embodiments, Poly-ICLC, an agonist of TLR3 and the RNA helicase-domains of MIDA5 and RIG3, has shown several desirable properties for a vaccine adjuvant. These properties include the induction of local and systemic activation of immune cells in vivo, production of stimulatory chemokines and cytokines, and stimulation of antigen-presentation by DCs. Furthermore, poly-ICLC can induce durable CD4⁺ and CD8⁺ responses in humans.

In certain embodiments, methods of identifying the most suitable peptides for preparing an immunogenic composition for a subject, comprises selecting from set given set of peptides the plurality of peptides capable of binding an HLA protein of the subject. In some embodiments, provided herein are methods of identifying a plurality of subject-specific peptides for preparing a subject-specific immunogenic composition, wherein the subject has a tumor and the subject-specific peptides are specific to the subject and the subject's tumor. In certain embodiments, the subject-specific peptides, each comprise a different tumor neo-epitope that is an epitope specific to the tumor of the subject and each binds an HLA protein of the subject, as provided by the methods for identifying HLA binding described herein.

In certain embodiments, the cell used in the method for determining HLA binding as described herein is an antigen-presenting cell.

### Neoantigens

In certain embodiments, the tumor antigens that bind to HLA alleles are identified by the methods described herein. In certain embodiments, the tumor antigens are neoantigens. In a further aspect, the disclosure provides methods for identifying tumor neoantigen-comprising peptides, wherein the methods comprise identifying for a given HLA allele, the peptides binding an HLA allele in a tumor cell from a tumor of a patient.

In some embodiments, the tumor antigen comprises KRAS, BRAF, FBWX7, FGFR3, IDH1, MUC4, NRAS, PIK3CA, PPP2R1A, PTEN, or TP53. In some embodiments, the tumor antigen comprises one or more mutations. In some embodiments, the one or more mutations comprises KRAS G12D, KRAS G12V, KRAS G12R, KRAS G12C, BRAF V600E, BRAF V600M, FBXW7 R465C, FBXW7 R465Q, FGFR3 S249Q, IDH1 R132C, MUC4 D3157N, NRAS Q61K, NRAS G545R, PIK3CA G545K, PIK3CA H1047R, PIK3CA R88Q, PPP2R1A P179R, PTEN R130G, PTEN R130Q, TP53 R175H, TP53 R248Q, TP53 R273H, TP53 R248Q, TP53 R273H, TP53 R282W, or TP53 R241Y.

In some embodiments, mutated epitopes are effective in inducing an immune response. In some embodiments, spontaneous tumor regression or long term survival correlate with CD8⁺ T-cell responses to mutated epitopes (Buckwalter and Srivastava P K. “It is the antigen(s), stupid” and other lessons from over a decade of vaccine therapy of human cancer. Seminars in immunology 20:296-300 (2008); Karanikas et al., High frequency of cytolytic T lymphocytes directed against a tumor-specific mutated antigen detectable with HLA tetramers in the blood of a lung carcinoma patient with long survival. Cancer Res. 61:3718-3724 (2001); Lennerz et al., The response of autologous T cells to a human melanoma is dominated by mutated neoantigens. Proc Natl Acad Sci USA. 102: 16013 (2005)) and that “immunoediting” can be tracked to alterations in expression of dominant mutated antigens in mice and man (Matsushita et al., Cancer exome analysis reveals a T-cell-dependent mechanism of cancer immunoediting Nature 482:400 (2012); DuPage et al., Expression of tumor-specific antigens underlies cancer immunoediting. Nature 482:405 (2012); and Sampson et al., Immunologic escape after prolonged progression-free survival with epidermal growth factor receptor variant III peptide vaccination in patients with newly diagnosed glioblastoma. J Clin Oncol. 28:4722-4729 (2010)).

In some embodiments, each tumor contains multiple, patient-specific mutations that alter the protein coding content of a gene. Such mutations create altered proteins, ranging from single amino acid changes (caused by missense mutations) to addition of long regions of novel amino acid sequence due to frame shifts, read-through of termination codons or translation of intron regions (novel open reading frame mutations; neoORFs). In some embodiments, these mutated proteins are valuable targets for the host's immune response to the tumor as, unlike native proteins, they are not subject to the immune-dampening effects of self-tolerance. In some embodiments, mutated proteins are more likely to be immunogenic and are also more specific for the tumor cells compared to normal cells of the patient. The mutated proteins can be referred to as neoantigens. The term “neoantigen” or “neoantigenic” means a class of tumor antigens that arises from a tumor-specific mutation(s) which alters the amino acid sequence of genome encoded proteins.

Embodiments disclosed herein provide a method of identifying peptides, e.g., neoantigens, including, but not limited to novel unannotated open reading frames (nuORFs), that are capable of eliciting a cancer specific T-cell response. Genomic aberrations in cancer cells give rise to mutant peptides (neoantigens) displayed on the human leukocyte antigen (HLA) molecules and recognized by T cells, thus triggering an immune response against cancer cells. In some embodiments, patients vaccinated with neoantigen-based peptides can display expanded neoantigen-specific T cells, and is a promising avenue for cancer treatment (Ott et al., An immunogenic personal neoantigen vaccine or patients with melanoma, Nature 2017 Jul. 13; 547(7662):217-221; Sahin et al., 2017 Personalized RNA mutanome vaccines mobilize poly-specific therapeutic immunity against cancer”, Nature, vol. 547, 2017, pages 222-226). In some embodiments, neoantigens can be predicted based on mutations detected by whole exome sequencing (WES). In some embodiments, their expression levels are estimated using mRNA sequencing (RNA-seq). Ribosome profiling (Ribo-seq) allows to monitor mRNA translation and has been used to predict a plethora of translated novel unannotated ORFs (nuORFs) (Fields et al., 2015. A Regression-Based Analysis of Ribosome-Profiling Data Reveals a Conserved Complexity to Mammalian Translation, Mol Cell, vol. 60, pages 816-827; Ji et al., 2015. Many IncRNAs, 5′UTRs, and pseudogenes are translated and some are likely to express functional proteins, ELIFE, vol. 4). Ribo-seq analysis of human fibroblasts infected with HSV-1 and HCMV has identified nuORFs that contribute peptides presented on major histocompatibility complex class I (MHC I) (Erhard et al., 2018. Improved Ribo-seq enables identification of cryptic translation events, Nat. Methods, vol. 15, no. 5, pages 363-366).

The methods disclosed herein can be used to select subject specific peptides that are presented by a tumor for any neoplasia. By “neoplasia” is meant any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphom′ (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mes′thelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal′carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma).

### Vaccines and Immunological Compositions

In certain embodiments, the peptides identified according to the present disclosure are used in a vaccine or immunological composition to treat any disease or condition described herein (e.g., tumor, autoimmunity, infection, transplant). The term “vaccine” or “immunological composition” are used interchangeably and are meant to refer in the present context to a pooled sample of one or more antigenic peptides, for example at least one, at least two, at least three, at least four, at least five, or more antigenic peptides. A “vaccine” is to be understood as including a protective vaccine, which is a composition for generating immunity for the prophylaxis and/or treatment of diseases (e.g., neoplasia/tumor). A “vaccine” is also to be understood as including a tolerizing vaccine, which is a composition for reducing immunity for the prophylaxis and/or treatment of diseases (e.g., autoimmune disease). A tolerizing vaccine may be formulated with antigenic epitopes specific for an allergen or for an autoimmunity antigen identified according to the present disclosure. In some embodiments, a protective vaccine may be formulated with antigenic epitopes specific for a pathogen or for a cancer cell. Accordingly, In some embodiments, vaccines can be medicaments which comprise antigens and are intended to be used in humans or animals for generating specific defense and protective substance by vaccination. A “vaccine composition” can include a pharmaceutically acceptable excipient, carrier or diluent.

In some embodiments, the vaccine may include one or more peptides identified according to the present disclosure. For example, 1 to 10 peptides. Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

In certain embodiments, a protective vaccine is used to treat cancer. Additional examples of cancers and cancer conditions that can be treated with the therapy of this document include, but are not limited to a patient in need thereof that has been diagnosed as having cancer, or at risk of developing cancer. The subject may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system′ (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites. Thus the population of subjects described herein may be suffering from one of the above cancer types. In other embodiments, the population of subjects may be all subjects suffering from solid tumors, or all subjects suffering from liquid tumors.

In some embodiments, cancers that can be treated using the therapy described herein may include among others cases which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are can be cancers that cannot be treated satisfactorily with other chemotherapeutics. In some embodiments, refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

In some embodiments, the therapy described herein is also applicable to the treatment of patients in need thereof who have not been previously treated.

In some embodiments, the therapy described herein is also applicable where the subject has no detectable neoplasia but is at high risk for disease recurrence.

In some embodiments, the therapy described herein is also applicable where the subject has undergone Autologous Hematopoietic Stem Cell Transplant (AHSCT), and in particular patients who demonstrate residual disease after undergoing AHSCT. The post-AHSCT setting is characterized by a low volume of residual disease, the infusion of immune cells to a situation of homeostatic expansion, and the absence of any standard relapse-delaying therapy. These features provide a unique opportunity to use the neoplastic vaccine or immunogenic composition compositions to delay disease relapse.

### In Vitro Peptide/Polypeptide Synthesis

Proteins or peptides disclosed herein may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. In some embodiments, the nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

In some embodiments, peptides can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963). In certain embodiments, neoantigenic peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Alternatively, a nucleic acid (e.g., a polynucleotide) encoding a neoantigenic peptide of the disclosure may be used to produce the neoantigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In some embodiments, in vitro translation is used to produce the peptide. Many exemplary systems exist that one skilled in the art could utilize (e.g., Retic Lysate IVT Kit, Life Technologies, Waltham, Mass.).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The neoantigenic peptides may be provided in the form of RNA or cDNA molecules encoding the desired neoantigenic peptides. One or more neoantigenic peptides of the disclosure may be encoded by a single expression vector.

In certain embodiments, the polynucleotides may comprise the coding sequence for the tumor specific neoantigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In certain embodiments, isolated nucleic acid molecules having a nucleotide sequence at least 60% identical, at least 65% identical, at least 70% identical, at least 75% identical, at least 80%) identical, at least 85% identical, at least 90% identical, at least 95% identical, or at least 96%), 97%, 98% or 99% identical to a polynucleotide encoding a tumor specific neoantigenic peptide of the present disclosure, can be provided. By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present disclosure, the parameters are set such that the percentage of identity is calculated over the full length of the reference nucleotide sequence and that gaps in homology of up to 5%>of the total number of nucleotides in the reference sequence are allowed.

Recombinant expression vectors may be used to amplify and express DNA encoding the tumor specific neoantigenic peptides. Recombinant expression vectors are replicable DNA constructs which have synthetic or cDNA-derived DNA fragments encoding a tumor specific neoantigenic peptide or a bioequivalent analog operatively linked to suitable transcriptional or translational regulatory elements derived from mammalian, microbial, viral or insect genes. In some embodiments, a transcriptional unit comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) can be operatively linked to DNA for a polypeptide if it is expressed as a precursor which participates in the secretion of the polypeptide; a promoter can be operatively linked to a coding sequence if it controls the transcription of the sequence; or a ribosome binding site can be operatively linked to a coding sequence if it is positioned so as to permit translation. Generally, operatively linked means contiguous, and in the case of secretory leaders, means contiguous and in reading frame. Structural elements intended for use in yeast expression systems can include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as MI 3 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23: 175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine (SEQ ID NO: 839), maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

### In Vivo Peptide/Polypeptide Synthesis

The present disclosure also contemplates the use of nucleic acid molecules as vehicles for delivering neoantigenic peptides/polypeptides to the subject in need thereof, in vivo, in the form of, e.g., DNA/RNA vaccines.

In certain embodiments, antigens may be administered to a patient in need thereof by use of a plasmid. In some embodiments, these can be plasmids which usually consist of a strong viral promoter to drive the in vivo transcription and translation of the gene (or complementary DNA) of interest (Mor, et al., (1995), Journal Immunol. 155 (4): 2039-2046). Intron A may sometimes be included to improve mRNA stability and hence increase protein expression (Leitner et al. (1997), Journal Immunol. 159 (12): 6112-6119). Plasmids also include a strong polyadenylation/transcriptional termination signal, such as bovine growth hormone or rabbit beta-globulin polyadenylation sequences Multi cistronic vectors are sometimes constructed to express more than one immunogen, or to express an immunogen and an immunostimulatory protein.

In some embodiments, a way of enhancing protein expression is by optimizing the codon usage of pathogenic mRNAs for eukaryotic cells. Another consideration is the choice of promoter. Such promoters may be the SV40 promoter or Rous Sarcoma Virus (RSV). Plasmids may be introduced into animal tissues by a number of different methods. The two most used approaches are injection of DNA in saline, using a standard hypodermic needle, and gene gun delivery. In some embodiments, injection in saline can be conducted intramuscularly (EVI) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose. Immune responses to this method of delivery can be affected by many factors, including needle type, needle alignment, speed of injection, volume of injection, muscle type, and age, sex and physiological condition of the animal being injected. In some embodiments, DNA or RNA that encodes a peptide disclosed herein can be administered to a subject.

Alternative delivery methods may include aerosol instillation of naked DNA on mucosal surfaces, such as the nasal and lung mucosa, and topical administration of pDNA to the eye and vaginal mucosa. Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex vivo Cytosolic Delivery of Functional Macromolecules to Immune Cells, Sharei et al., PLOS ONE DOI: 10.1371/journal.pone.O1 18803 Apr. 13, 2015).

In certain embodiments, a neoplasia vaccine or immunogenic composition may include separate DNA plasmids encoding, for example, one or more neoantigenic peptides/polypeptides as identified in according to the disclosure. As discussed herein, the exact choice of expression vectors can depend upon the peptide/polypeptides to be expressed and is well within the skill of the ordinary artisan. The expected persistence of the DNA constructs (e.g., in an episomal, non-replicating, non-integrated form in the muscle cells) is expected to provide an increased duration of protection.

One or more antigenic peptides of the disclosure may be encoded and expressed in vivo using a viral based system (e.g., an adenovirus system, an adeno associated virus (AAV) vector, a poxvirus, or a lentivirus). In one embodiment, the neoplasia vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7). Plasmids that can be used for adeno associated virus, adenovirus, and lentivirus delivery have been described previously. The peptides and polypeptides of the disclosure can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. In some embodiments, this approach can involve the use of a vector to express nucleotide sequences that encode the peptide of the disclosure. Upon introduction into an acutely or chronically infected host or into a noninfected host, the vector can express the immunogenic peptide, and thereby elicits a host CTL response.

Among vectors that may be used in the practice of the disclosure, integration in the host genome of a cell is possible with retrovirus gene transfer methods, often resulting in long term expression of the inserted transgene. In certain embodiments, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. In some embodiments, cell type specific promoters can be used to target expression in specific cell types. Lentiviral vectors are retroviral vectors (and hence both lentiviral and retroviral vectors may be used in the practice of the disclosure). Moreover, In some embodiments, lentiviral vectors can be preferred as they are able to transduce or infect non-dividing cells and may produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the disclosure include those based upon murine leukemia virus (MuLV), gibbon ape leukemia virus (GaLV), Simian Immunodeficiency virus (SIV), human immunodeficiency virus (HIV), and combinations thereof.

Also useful is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the disclosure contemplates amongst vector(s) useful in the practice of the disclosure: viral vectors, including retroviral vectors and lentiviral vectors.

One of skill in the art can determine suitable dosage. Suitable dosages for a virus can be determined empirically. Also useful in the practice of the disclosure is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848).

In some embodiments, an adenovirus vector used can be selected from the group consisting of the Ad5, Ad35, Adl 1, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285). Ad35 vectors are described in U.S. Pat. Nos. 6,974,695, 6,913,922, and 6,869,794. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present disclosure. Furthermore, “gutless” adenovirus vectors, in which all viral genes are deleted, can also be used in accordance with the present disclosure. In some embodiments, such vectors require a helper virus for their replication and require a special human 293 cell line expressing both Ela and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present disclosure and can even be used for co-delivery of a large number of heterologous inserts/genes.

In one embodiment, the viral vector is an adenovirus vector, an adeno-associated viral vector (AAV), or derivatives thereof. The adeno-associated viral vector comprises AAV serotypes 1, 2, 3, 4, 5, 6, 7, 8, 9, 10, 11, 12, 13, 14, 15, 16, DJ, DJ/8 or pseudotypes thereof. In one embodiment, the AAV can be AAV1, AAV2, AAV5 or any combination thereof. In some embodiments, one can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. In some embodiments, AAV8 can be useful for delivery to the liver.

In another embodiment effectively activating a cellular immune response for a neoplasia vaccine or immunogenic composition can be achieved by expressing the relevant antigens in a vaccine or immunogenic composition in a non-pathogenic microorganism. Well-known examples of such microorganisms include but are not limited to Mycobacterium bovis BCG, Salmonella and Pseudomona (See, U.S. Pat. No. 6,991,797). In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (see e.g., Verardi et al., Hum Vaccin Immunother. 2012 July; 8(7):961-70; and Moss, Vaccine. 2013; 31(39): 4220-4222).

In some embodiments, poxviruses that may be used in the practice of the disclosure, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC. 1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature.

In one embodiment recombinant viral particles of the vaccine or immunogenic composition are administered to patients in need thereof. Dosages of expressed neoantigen can range from a few to a few hundred micrograms, e.g., 5 to 500 g. The vaccine or immunogenic composition can be administered in any suitable amount to achieve expression at these dosage levels. The viral particles can be administered to a patient in need thereof or transfected into cells in an amount of about at least 10³ pfu; thus, the viral particles are preferably administered to a patient in need thereof or infected or transfected into cells in at least about 10⁴ pfu to about 10⁶ pfu; however, a patient in need thereof can be administered at least about 10⁸ pfu or at least about 10⁷ pfu to about 10⁹ pfu. Doses as to NYVAC are applicable as to ALVAC, MVA, MVA-BN, and avipoxes, such as canarypox and fowlpox.

### Pharmaceutical Compositions/Methods of Delivery

In certain embodiments, a pharmaceutical composition comprises an effective amount of one or more antigenic peptides as described herein (including a pharmaceutically acceptable salt, thereof), optionally in combination with a pharmaceutically acceptable carrier, excipient or additive.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neoantigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

When administered as a combination, the therapeutic agents, for example, the neoantigenic peptides, can be formulated as separate compositions that are given at the same time or different times, or the therapeutic agents can be given as a single composition.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

### Combination Therapies

The disclosure also contemplates the combination of the composition of the present disclosure with other drugs and/or in addition to other treatment regimens or modalities such as surgery. When the composition of the present disclosure is used in combination with known therapeutic agents the combination may be administered either in sequence (either continuously or broken up by periods of no treatment) or concurrently or as an admixture. For example, in the case of cancer chemotherapeutic agents may be administered as part of the combination therapy.

In certain embodiments, the KRAS and/or mKRAS peptides are administered in conjunction with a cancer therapy. In some embodiments, the compositions disclosed herein are administered in conjunction with a cancer therapy. In some embodiments, an immunogenic peptide comprising at least 90%, 95%, 98%, or 99% sequence identity to the amino acid sequence of any one of SEQ ID NOs: 1-838 can be administered in conjunction with a cancer therapy. In some embodiments, one or more immunogenic peptides comprise at least a 75% sequence identity to any one or more of SEQ ID NOs: 1-838 can be administered with a cancer therapy. In certain embodiments, the immunogenic peptides comprise any one or more of SEQ ID NOs: 1-838 can be administered with a cancer therapy. In certain embodiments, the immunogenic peptides comprises an amino acid sequence having at least 95% sequence identity to any one or more of SEQ ID NOs: 1-838 having one or more conservative substitutions can be administered with a cancer therapy. In some embodiments, the immunogenic peptides comprising the amino acid sequence of any one of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834 can be administered in conjunction with a cancer therapy. In some embodiments, the immunogenic peptides comprise the amino acid sequence of SEQ ID NO: 2, SEQ ID NO: 3, SEQ ID NO: 833, or SEQ ID NO: 834 and can be administered with a cancer therapy. As used herein, the term “cancer therapy” refers to a therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN™), anti-CD20 antibodies, an epidermal growth factor receptor (EGFR) antagonist (e.g., a tyrosine kinase inhibitor), HER1/EGFR inhibitor (e.g., erlotinib (TARCEVA™)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include Erlotinib (TARCEVA™, Genentech/OSI Pharm.), Bortezomib (VELCADE™, Millennium Pharm.), Fulvestrant (FASLODEX™, Astrazeneca), Sutent (SU11248, Pfizer), Letrozole (FEMARA™, Novartis), Imatinib mesylate (GLEEVEC™ Novartis), PTK787/ZK 222584 (Novartis), Oxaliplatin (Eloxatin™, Sanofi), 5-FU (5-fluorouracil), Leucovorin, Rapamycin (Sirolimus, RAPAMUNE™, Wyeth), Lapatinib (GSK572016, GlaxoSmithKline), Lonafarnib (SCH 66336), Sorafenib (BAY43-9006, Bayer Labs.), and Gefitinib (IRESSA™, Astrazeneca), AG1478, AG1571 (SU 5271; Sugen), alkylating agents such as Thiotepa and CYTOXAN™ cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin omega 1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK™ polysaccharide complex (JHS Natural Products, Eugene, Oreg.); razoxane; rhizoxin; sizofuran; spirogermanium; tenuazonic acid; tr iaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamidc; thiotcpa; taxoids, e.g., TAXOL™ paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulene Rorer, Antony, France); chloranbucil; GEMZAR™ gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE™ vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX™ (tamoxifen)), raloxifene, droloxifene, 4-hydroxytamoxifen, trioxifene, keoxifene, LY117018, onapristone, and FARESTON™ (toremifene); (ii) aromatase inhibitors that inhibit the enzyme aromatase, which regulates estrogen production in the adrenal glands, such as, for example, 4(5)-imidazoles, aminoglutethimide, MEGASE™ (megestrol acetate), AROMASIN™ (exemestane), formestanie, fadrozole, RIVISOR™ (vorozole), FEMARA™ (letrozole), and ARIMIDEX™ (anastrozole); (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in aberrant cell proliferation, such as, for example, PKC-alpha, Ralf and H-Ras; (viii) ribozymes such as a VEGF expression inhibitor (e.g., ANGIOZYME™ (ribozyme)) and a HER2 expression inhibitor; (ix) vaccines such as gene therapy vaccines, for example, ALLOVECTIN™ vaccine, LEUVECTIN™ vaccine, and VAXID™ vaccine; PROLEUKIN™ rIL-2; LURTOTECAN™ topoisomerase 1 inhibitor; ABARELIX™ rmRH; (x) anti-angiogenic agents such as bevacizumab (AVASTIN™, Genentech); and (xi) pharmaceutically acceptable salts, acids or derivatives of any of the above.

In various embodiments, the cancer therapeutic is an immunotherapy selected from the group comprising oncolytic virus, bacteria, oncolytic bacteria or other bacterial compositions, Bacillus Calmette-Guerin (BCG), a microbiome modulator, and/or a toll-like receptor (TLR) agonist. In various embodiments, the TLR agonist is a TLR3, TLR4, TLR5, TLR7, TLR8, TLR9, TLR10, TLR11, TLR12, and/or TLR13 agonist. In various embodiments, the TLR agonist is derived from virus, plants, bacteria and/or made synthetically. In various embodiments, the immunotherapy is a is a stimulator of interferon genes (STING) pathway modulator.

It will be appreciated by those skilled in the art of cancer immunotherapy that other complementary immune therapies may be added to the regimens described above to further enhance their efficacy including but not limited to GM-CSF to increase the number of myeloid derived innate immune system cells, low dose cyclophosphamide or PI3K inhibitors (e.g., PI3K delta inhibitors) to eliminate T regulatory cells that inhibit innate and adaptive immunity and 5FU (e.g., capecitabine), PI3K inhibitors or histone deacetylase inhibitors to remove inhibitory myeloid derived suppressor cells. For example, PI3K inhibitors include, but are not limited to, LY294002, Perifosine, BKM120, Duvelisib, PX-866, BAY 80-6946, BEZ235, SF1126, GDC-0941, XL147, XL765, Palomid 529, GSK1059615, PWT33597, IC87114, TG100-15, CAL263, PI-103, GNE-477, CUDC-907, and AEZS-136. In some aspects, the PI3K inhibitor is a PI3K delta inhibitor such as, but not limited to, Idelalisib, RP6530, TGR1202, and RP6503. The immunotherapy may also comprise the administration of an interleukin such as IL-2, or an interferon such as INFα.

In certain embodiments, the KRAS and/or mKRAS peptides are administered with one or more immune checkpoint modulators. In some embodiments, the composition disclosed herein can be administered with an immune checkpoint modulator. In some embodiments, an immunogenic peptide comprising at least 90%, 95%, 98%, 99% or 100% sequence identity to the amino acid sequence of any one of SEQ ID NOs: 1-838 can be administered in conjunction with a checkpoint modulator. In some embodiments, one or more immunogenic peptides comprise at least a 75% sequence identity to any one or more of SEQ ID NOs: 1-838 can be administer with an immune checkpoint modulator. In certain embodiments, the immunogenic peptides comprise any one or more of SEQ ID NOs: 1-838 can be administered with an immune checkpoint modulator. In some embodiments, an immunogenic peptide comprising the amino acid sequence of any one of SEQ ID NOs: 2, 3, 5-12, 14-21, 23-30, 32-39, 41-48, 50-57, 59-66, 68-75, 77-84, 86-93, 85-99, 101-105, 107-111, 113-117, 119-123, 125-129, 131-135, 137-141, 143-146, 148-152, 154-162, 164-168, 170-179, 181-185, 187-196, 198-202, 204-213, 215-219, 221-230, 232-236, 238-244, 246-252, 254-260, 262-268, 270-276, 278-284, 286-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 532-536, 538-542, 544-548, 550-554, 556-560, 562-566, 568-573, 585-580, 582-587, 589-594, 596-601, 603-607, 609-613, 615-619, 621-625, 627-631, 633-638, 340-644, 646-651, 653-657, 659-664, 666-670, 672-677, 679-683, 685-690, 692-696, 698-703, 705-711, 713-718, 720-726, 728-733, 735-741, 743-748, 750-756, 758-763, 765-771, 773-777, 779-783, 785-789, 791-795, 797-801, 803-807, 809-813, 815-819, 821-825, 827-831, 833, or 834 can be administered in conjunction with a checkpoint modulator. In certain embodiments, the immunogenic peptides comprises an amino acid sequence having at least 95% sequence identity to any one or more of SEQ ID NOs: 1-838 having one or more conservative substitutions can be administered with an immune checkpoint modulator. In some embodiments, the immunogenic peptides comprise the amino acid sequence of SEQ ID NO: 2, SEQ ID NO: 3, SEQ ID NO: 833, or SEQ ID NO: 834 can be administered with an immune checkpoint modulator. Immune checkpoints refer to inhibitory pathways of the immune system that are responsible for maintaining self-tolerance and modulating the duration and amplitude of physiological immune responses. Examples of checkpoint inhibitor include, without limitation an inhibitor of: PD-1, PD-L1, PD-L2, CTLA4, TIM-3, LAG-3, CEACAM-1, CEACAM-5, VISTA, BTLA, TIGIT, LAIR1, CD 160, 2B4 or TGFR-β.

The term “checkpoint inhibitor” means a group of molecules on the cell surface of CD4+ and/or CD8⁺ T cells that fine-tune immune responses by down-modulating or inhibiting an anti-tumor immune response. Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPα (CD47), CD48, 2B4 (CD244), B7.1, B7.2, ILT-2, ILT-4, TIGIT, and A2aR (see, for example, WO 2012/177624). “Anti-immune checkpoint inhibitor therapy” refers to the use of agents that inhibit immune checkpoint inhibitors. Inhibition of one or more immune checkpoint inhibitors can block or otherwise neutralize inhibitory signaling to thereby upregulate an immune response in order to more efficaciously treat cancer. Exemplary agents useful for inhibiting immune checkpoint inhibitors include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint inhibitor nucleic acids, or fragments thereof. Exemplary agents for upregulating an immune response include antibodies against one or more immune checkpoint inhibitor proteins block the interaction between the proteins and its natural receptor(s); a non-activating form of one or more immune checkpoint inhibitor proteins (e.g., a dominant negative polypeptide); small molecules or peptides that block the interaction between one or more immune checkpoint inhibitor proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint inhibitor nucleic acid transcription or translation; and the like. Such agents can directly block the interaction between the one or more immune checkpoint inhibitors and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or used in combination.

In some embodiments, such therapy involves blockade of programmed cell death 1 (PD-1). In some embodiments, such therapy involves treatment with an agent that interferes with an interaction involving PD-1 (e.g., with PD-L1). In some embodiments, such therapy involves administration of an antibody agent that specifically interacts with PD-1 or with PD-L1. In some embodiments, such therapy involves administration of one or more of nivolumab (BMS-936558, MDX-1106, ONO-4538, a fully human Immunoglobulin G4 (IgG4) monoclonal PD-1 antibody), pembrolizumab (MK-3475, a humanized monoclonal IgG4 anti-PD-1 antibody), BMS-936559 (a fully human IgG4 PD-L1 antibody), MPDL3280A (a humanized engineered IgG1 monoclonal PD-L1 antibody) and/or MEDI4736 (a humanized engineered IgG1 monoclonal PD-L1 antibody).

Each embodiment disclosed herein is contemplated as being applicable to each of the other disclosed embodiments. Thus, all combinations of the various elements described herein are within the scope of the disclosure.

This disclosure is further illustrated by the following examples which should not be construed as limiting. The contents of all references, patents, and published patent applications cited throughout this application, as well as the figures and the sequence listing, are hereby incorporated by reference.

### EXAMPLES

### Example 1: Heteroclitic Neoepitope Vaccines Derived from Computational Structural Modeling

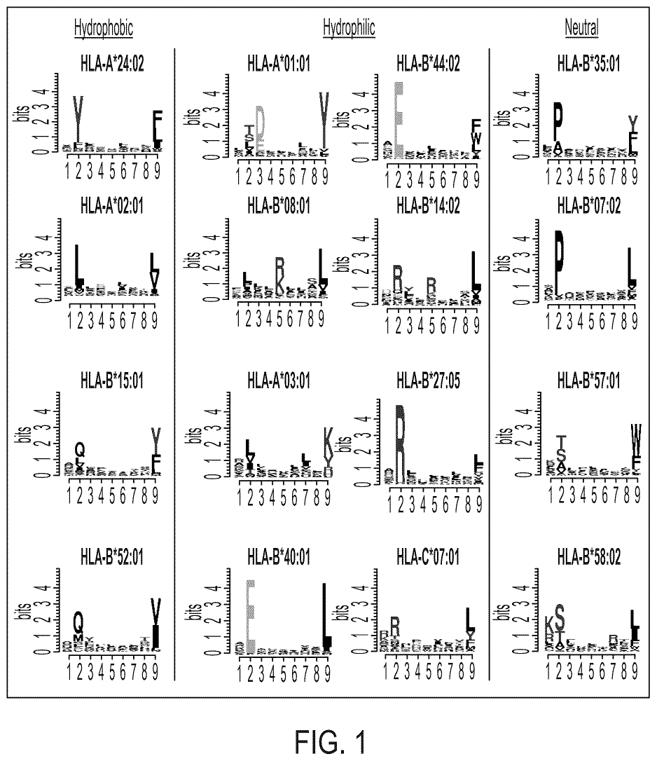

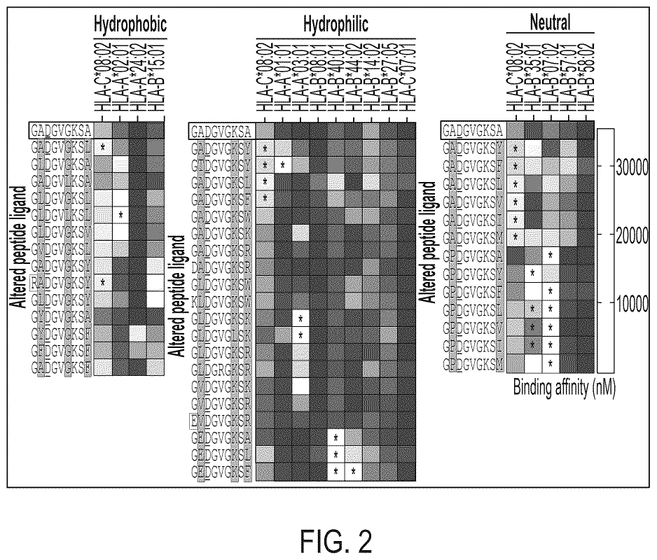

A library of KRAS G12D heteroclitic epitopes was generated for structural analysis in a panel of 18 HLAs representing >90% of the global population. The consensus binding motif for epitopes of each HLA showed distinct anchor residue binding motifs subdivided into hydrophobic, hydrophilic, or neutral binding anchors (FIG. 1). Major anchor residues were predominately at position 2, 3, 5, and 9. Some alleles had additional minor anchor residues at positions 1, 4, 6, and 7. Using the KRAS G12D 9-mer epitopes GADGVGKSA (SEQ ID NO: 639) and VVGADGVGK (SEQ ID NO: 214), a library of amino acid modified peptides was generated with every combination of anchor residue amino acid for each HLA subgroup. As proof of concept, NetMHC prediction of heteroclitic epitope binding affinity within each HLA subgroup was performed in silico and indicated an increase in binding affinity in some epitopes for all HLA types (FIG. 2, GADGVGKSA (SEQ ID NO: 639) shown only). Consistent with previous reports, parental GADGVGKSA (SEQ ID NO: 639) was not predicted to bind HLA-C*08:02, emphasizing the limitation of using predicted binding affinity to identify neoantigen targets.

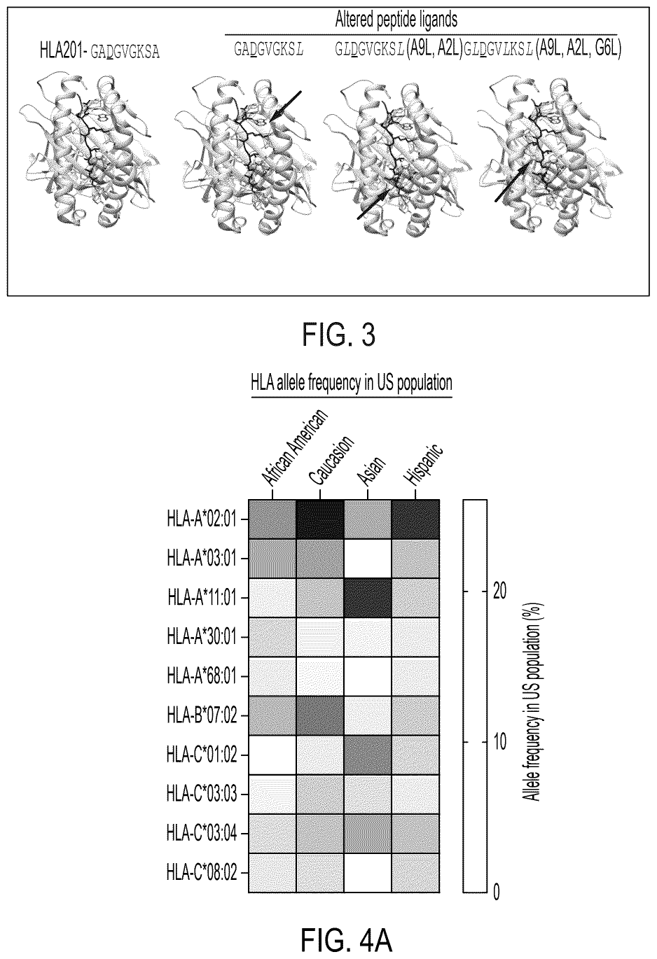

To demonstrate the structural modelling approach herein, each peptide was first modelled onto HLA-A*02:01 to investigate peptide-HLA interactions (FIG. 3). A Multiple Sequence Comparison by Log-Expectation (MUSCLE) peptide sequence alignment was performed, which takes into consideration positional sequence similarity, of the KRAS G12D epitopes with all epitopes that bind each HLA and have a crystal structure available. Using the HLA-peptide structure with the greatest sequence similarity, the KRAS neoepitopes were modeled into the HLA cleft by introducing amino acid rotamers with the lowest energy confirmation at each position. The resulting HLA-KRAS neoepitope complexes were structurally validated using the root-mean-square deviation analysis (Ca-RMSD) to ensure no major destabilizing structural changes have occurred to overall HLA cleft. High-resolution, Monte Carlo with minimization docking of the peptide to the HLA cleft was performed to identify the lowest full-atom energy conformation. The energetic-optimized structures are used to introduce each anchor residue modification found in the heteroclitic epitope library (FIG. 2 and FIG. 3). Using this approach, an increase in the number of contacts between peptides and HLA molecules was found after each amino acid anchor residue substitution at position 2, 6, and 9, demonstrating the ability to detect structural interaction differences in HLA-heteroclitic peptide interactions (FIG. 3).

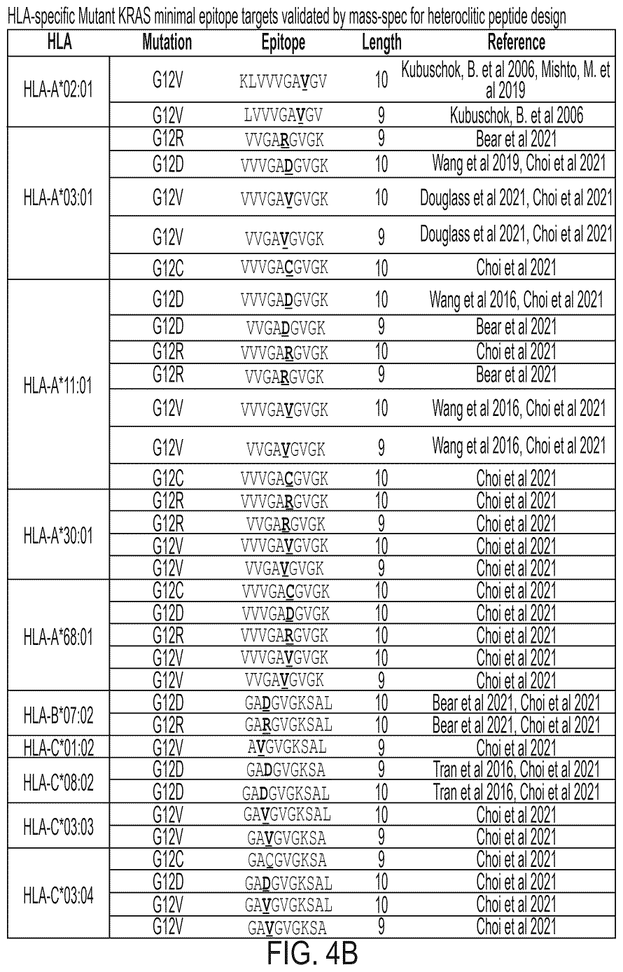

HLA-specific mutant KRAS minimal epitope targets were validated by mass-spectrometry for heterolytic peptide design (FIG. 4A-FIG. 4B).

These neoepitope-HLA structural models were used to analyze the stereochemical changes between the parental peptide and the modified peptides. Structural parameters such as contacts between peptide and HLA cleft residues, solvent-assessable surface area (SASA), peptide rigidity as a measure of CaRMSD of the top 10 predicted structural models for each peptide-HLA complex, surface hydrophobicity, and electrostatic potential. These measurements are compared to modelled parental neoepitopes in each HLA molecule.

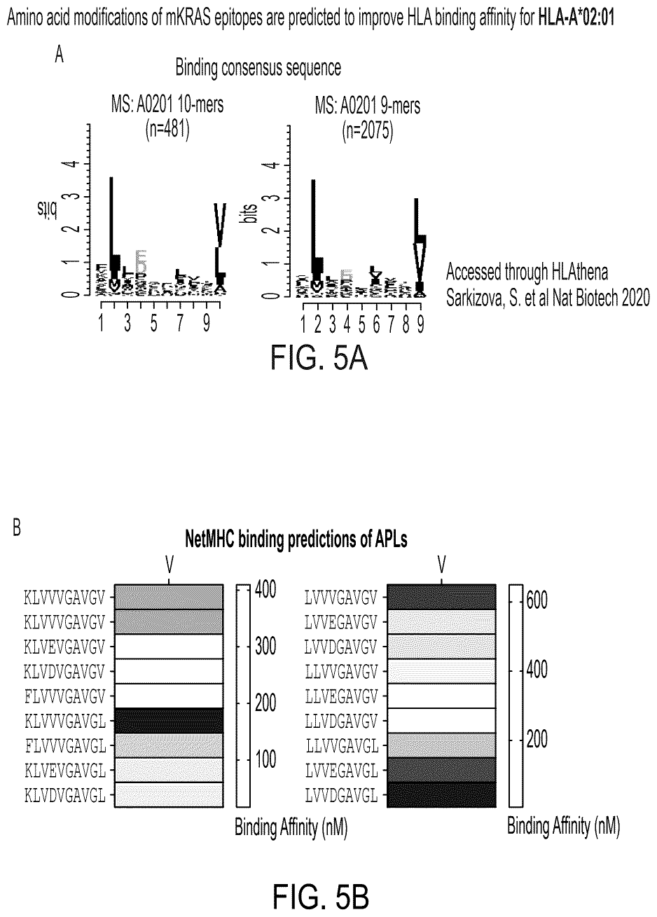

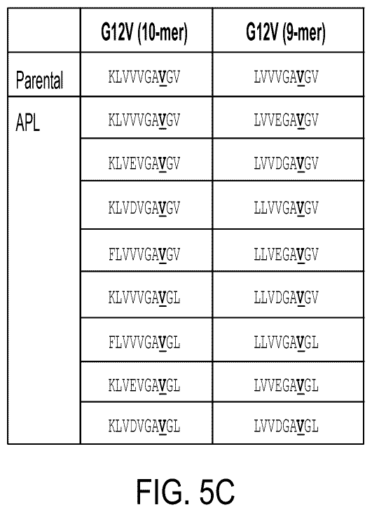

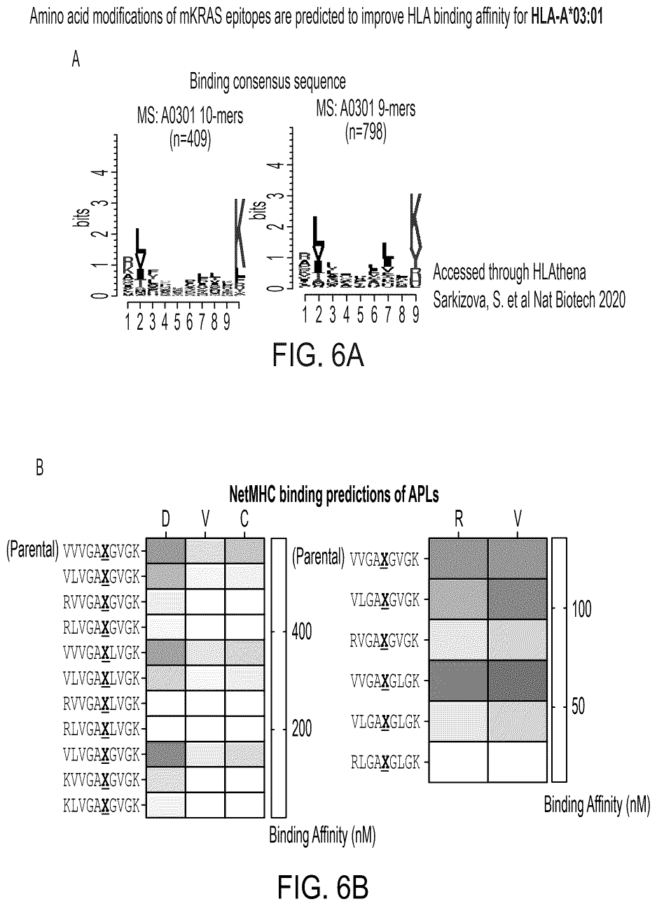

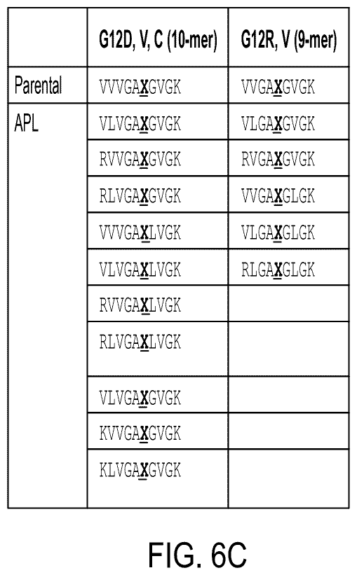

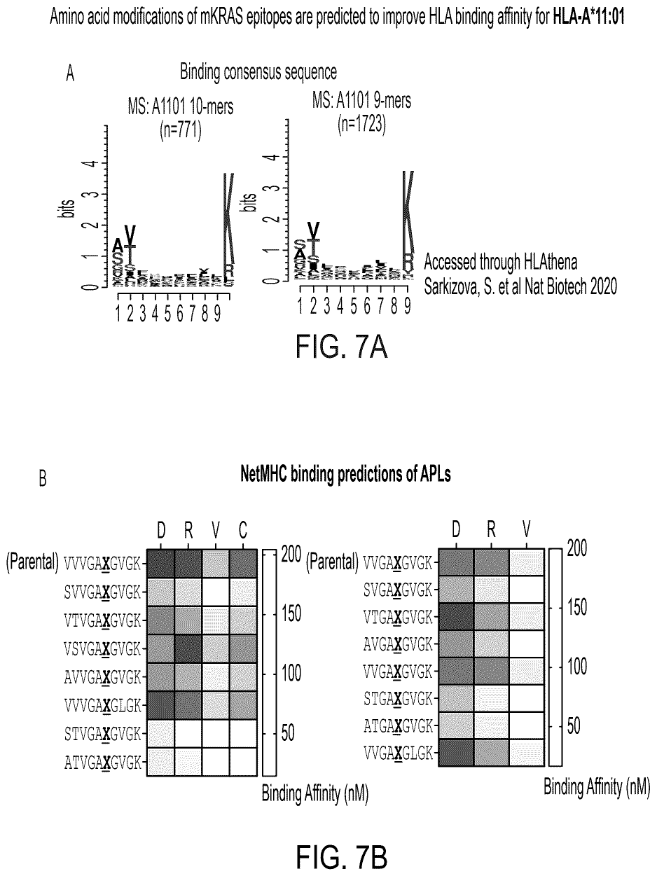

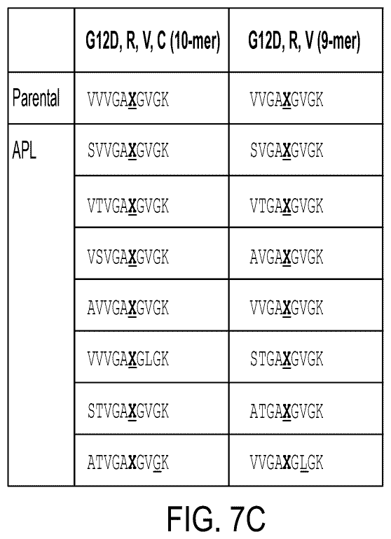

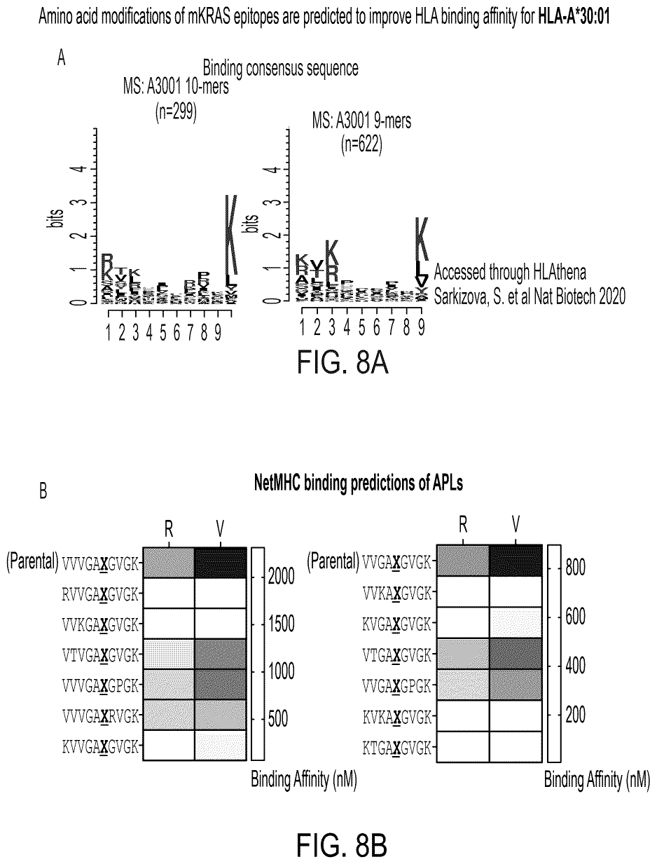

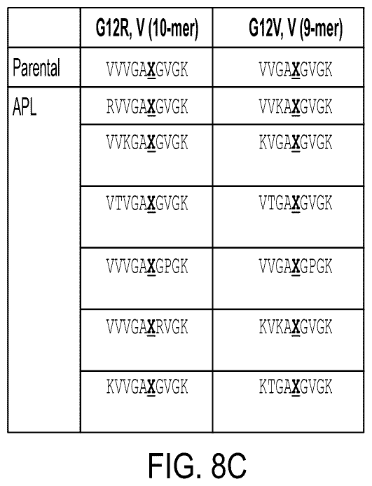

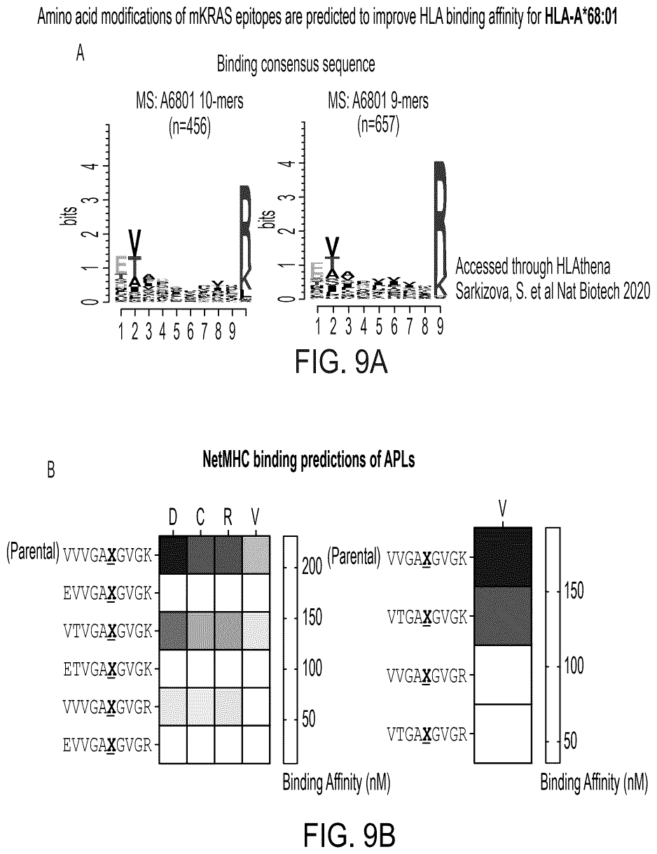

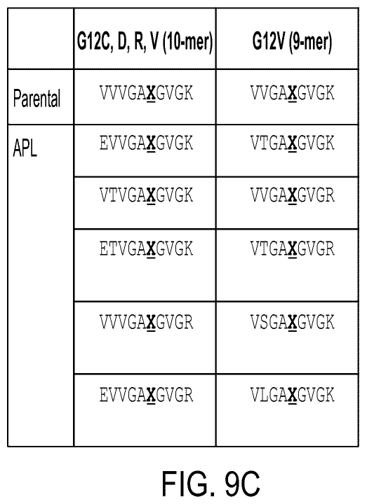

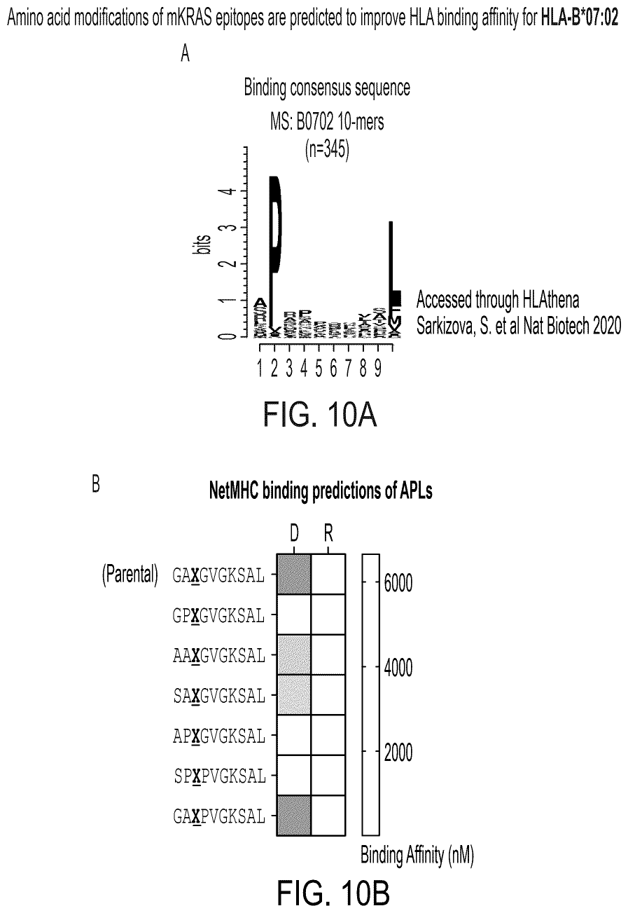

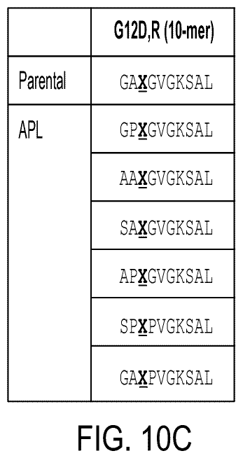

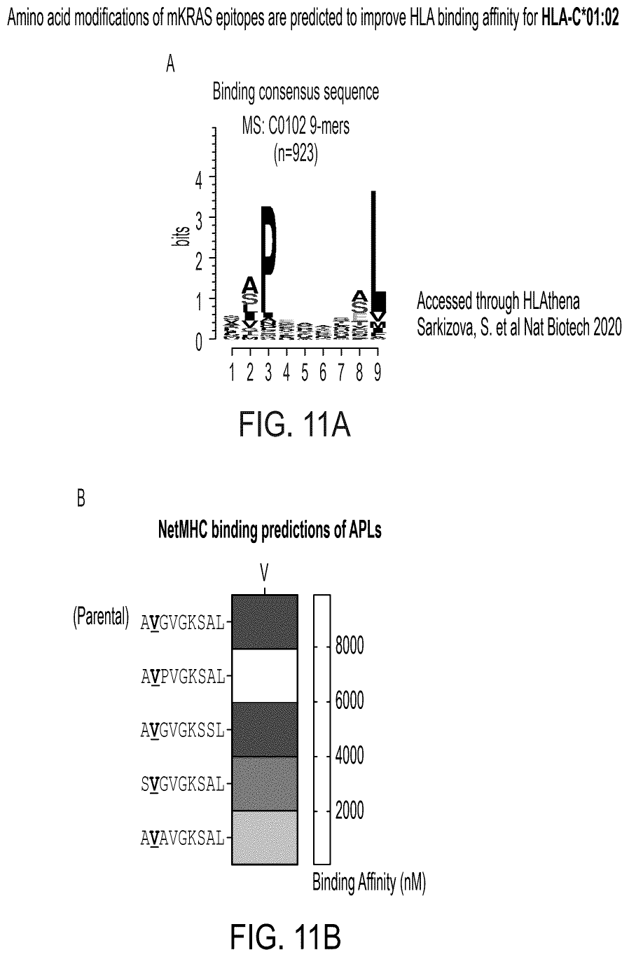

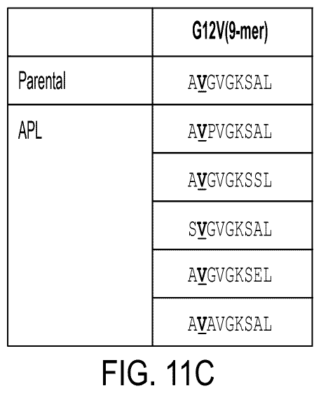

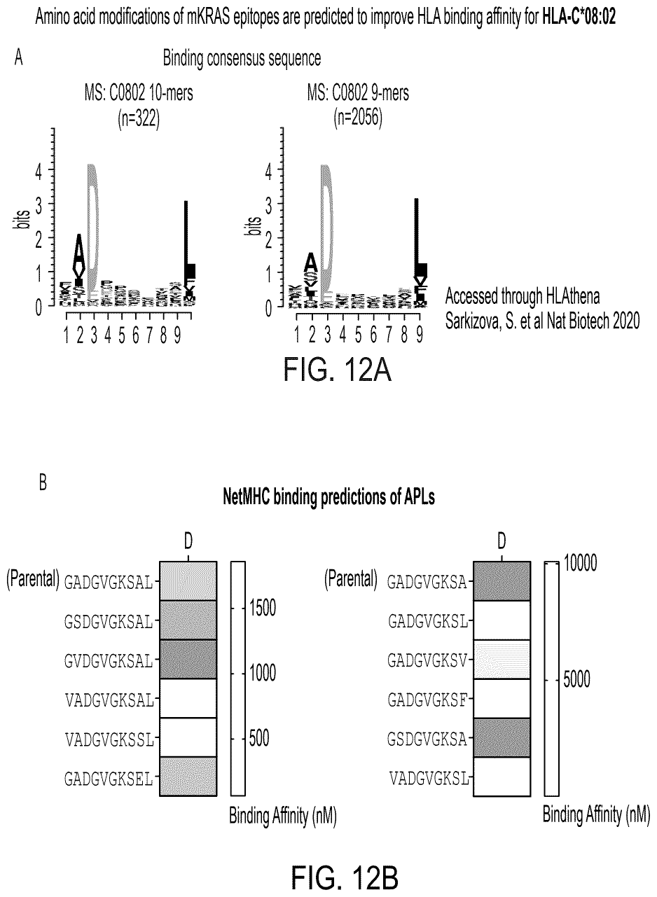

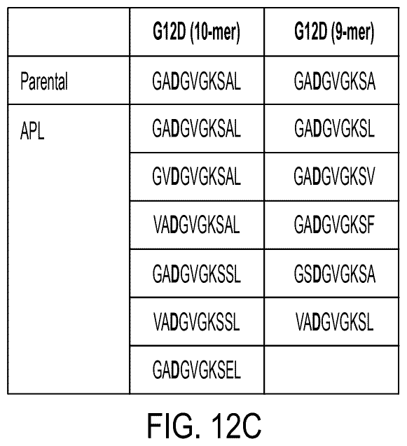

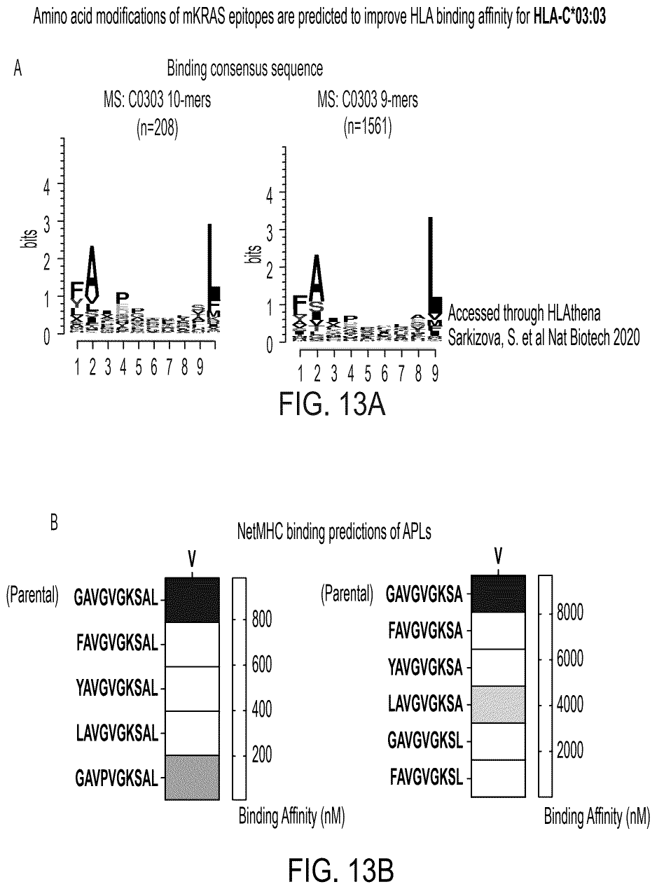

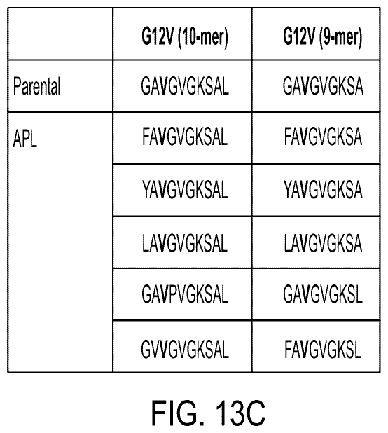

Using the neoepitope-HLA structural models, amino acid modifications of mKRAS epitopes with improved HLA binding affinity were predicted for HLA-A*02:01 (FIG. 5A-FIG. 5C), HLA-A*03:01 (FIG. 6A-FIG. 6C), HLA-A*aa:01 (FIG. 7A-FIG. 7C), HLA-A*30:01 (FIG. 8A-FIG. 8C), HLA-A*68:01 (FIG. 9A-FIG. 9C), HLA-B*07:02 (FIG. 10A-FIG. 10C), HLA-C*01:02 (FIG. 11A-FIG. 11C), HLA-C*08:02 (FIG. 12A-FIG. 12C), and HLA-C*03:03 (FIG. 13A-FIG. 13C).

Predicted binding affinity (nM) of altered peptide ligands (APL) of parental G12V 10-mer and 9-mer and APLs to HLA-A*02:01 was generated (FIG. 5B).

Predicted binding affinity (nM) of altered peptide ligands (APL) of parental 10-mer (KRAS G12D, G12V, G12C) and 9-mer (KRAS G12R, G12V) and APLs to HLA-A*03:01 was generated (FIG. 6B).

Predicted binding affinity (nM) of altered peptide ligands (APL) of parental 10-mer (KRAS G12D, G12R, G12V, G12C) and 9-mer (KRAS G12D, G12R, G12V) and APLs to HLA-A*11:01 was generated (FIG. 7B).

Predicted binding affinity (nM) of altered peptide ligands (APL) of parental 10-mer (KRAS G12R, G12V) and 9-mer (KRAS G12R, G12V) and APLs to HLA-A*30:01 was generated (FIG. 8B).

Predicted binding affinity (nM) of altered peptide ligands (APL) of parental 10-mer (KRAS G12D G12C, G12R, G12V) and 9-mer (KRAS G12V) and APLs to HLA-A*68:01 was generated (FIG. 9B).

Predicted binding affinity (nM) of altered peptide ligands (APL) of parental 10-mer (KRAS G12D and G12R) and APLs to HLA-B*07:02 was generated (FIG. 10B).

Predicted binding affinity (nM) of altered peptide ligands (APL) of parental 9-mer (KRAS G12V) and APLs to HLA-C*01:02 was generated (FIG. 11B).

Predicted binding affinity (nM) of altered peptide ligands (APL) of parental 10-mer (KRAS G12D) and 9-mer (KRAS G12D) and APLs to HLA-C*08:01 was generated (FIG. 12B).

Predicted binding affinity (nM) of altered peptide ligands (APL) of parental 10-mer (KRAS G12V) and 9-mer (KRAS G12V) and APLs to HLA-C*03:03 was generated (FIG. 13B).

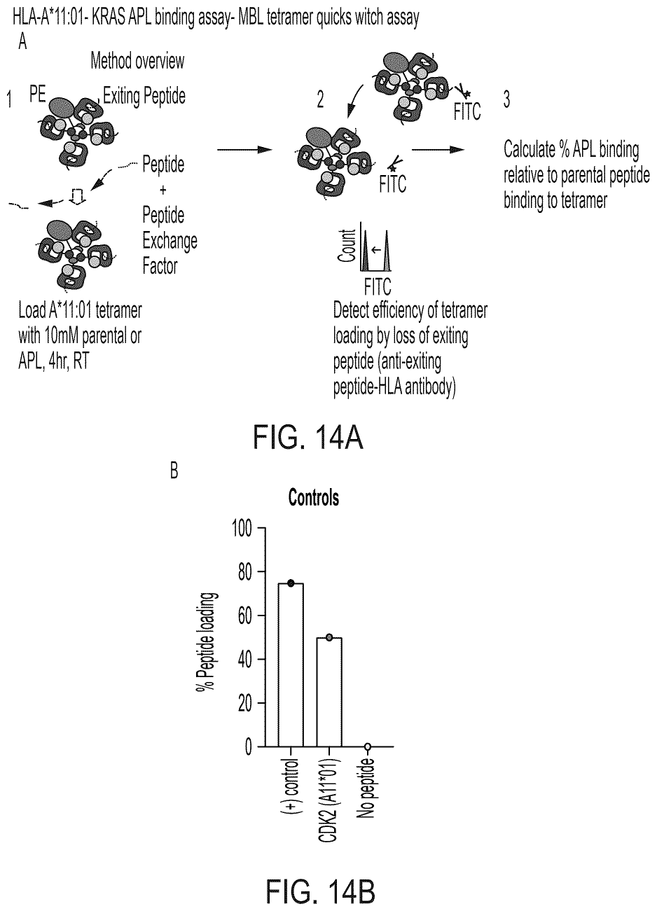

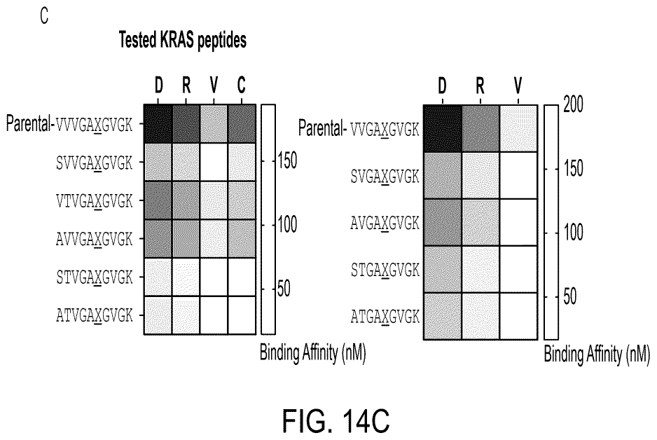

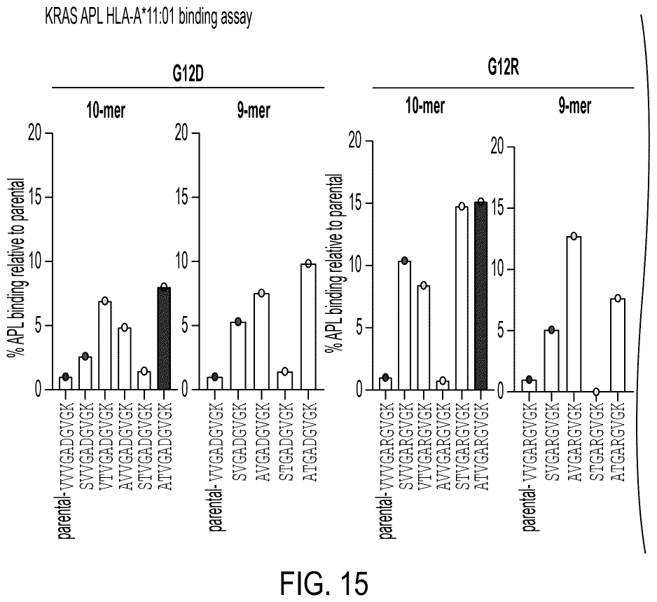

HLA-A*11:01-KRAS APL binding was tested using MBL tetramer quick switch assay. HLA-A*11:01 tetramers were incubated with 10 mM KRAS parental epitope or APL at room temperature for 4 hours. Peptide loading was then quantified by flow cytometry analysis with anti-exiting peptide antibody. % APL binding relative to parental peptide binding to tetramer was calculated (FIG. 14A). NetMHC prediction was used to predict binding affinities for HLA-A*11:01-specific KRAS peptides and APLs tested with this assay.

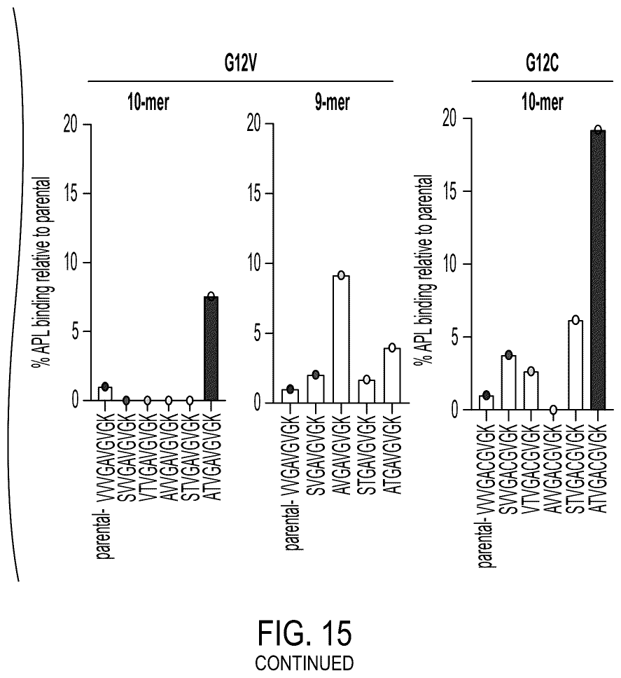

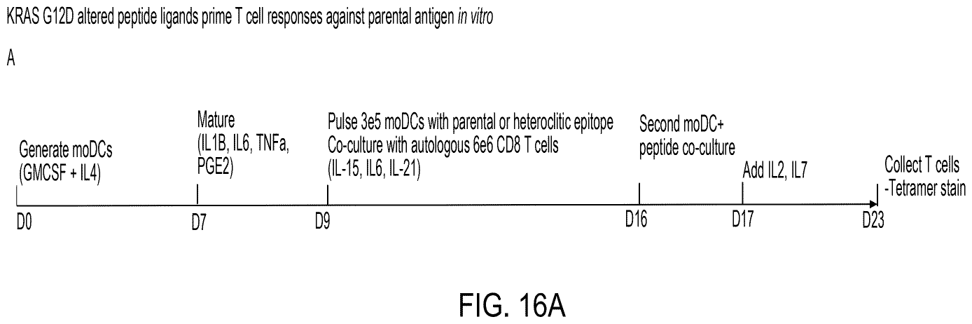

Percent binding of KRAS G12D, G12R, G12V, and G12C 10- and 9-mer parental epitopes and altered peptide ligands is shown in FIG. 15. APLs with greater binding percentage relative to parental are shaded in gray.

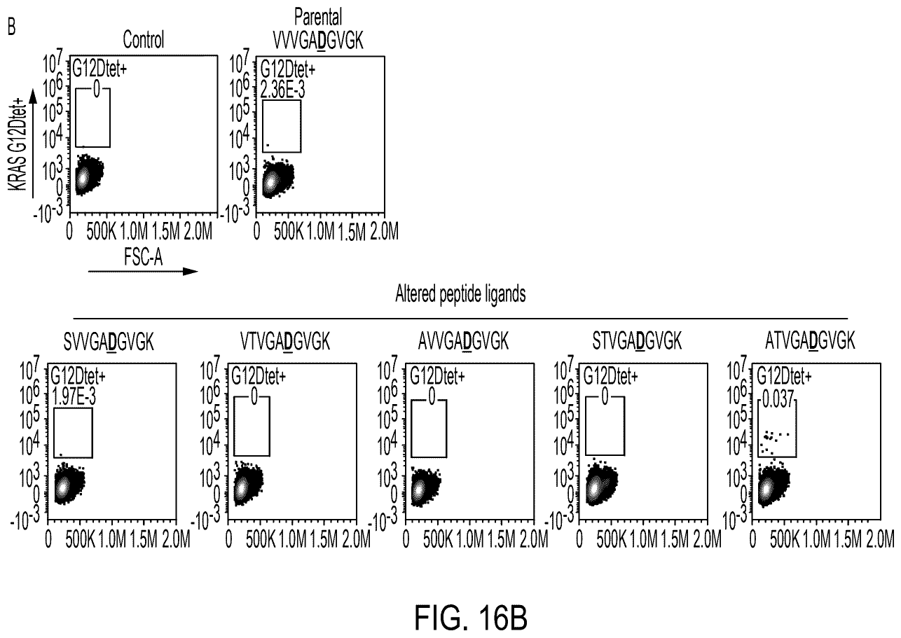

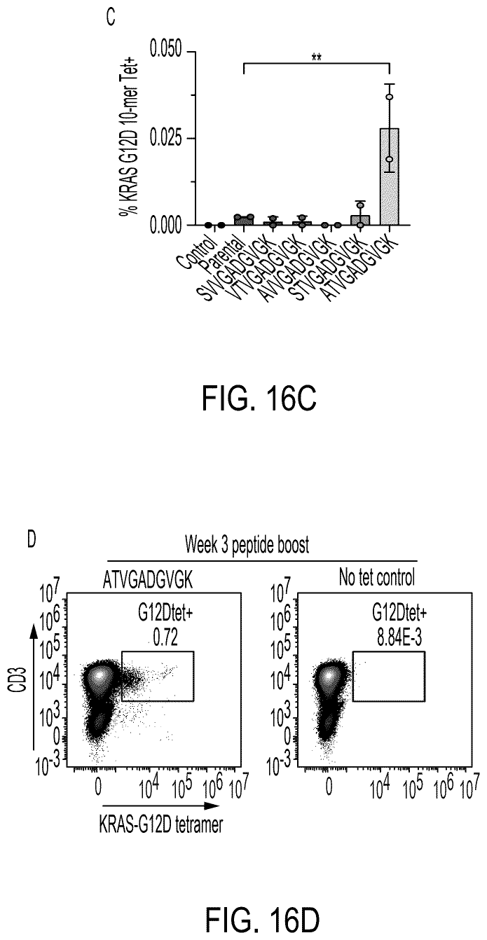

The ability of KRAS G12D APLs to prime T cell responses against parental antigen was tested in vitro. An in vitro immunogenicity assay was conducted for KRAS G12D altered peptide ligands in an HLA-A*11:01 healthy donor. Healthy donor dendritic cells were generated by adherence of monocytes to flasks and differentiated in 800 U/mL hGMCSF and 400 IU/mL hIL-4 for 7 days. Dendritic cells were then matured in hIL-1B, IL-6, TNFα, and PGE2 for 2 days. Mature dendritic cells were then collected and pulsed with 40 μg/mL parental or altered peptide ligand for 2 hrs. Pulsed dendritic cells were washed twice with PBS and then co-cultured with autologous CD8⁺ T cells in the presence of hIL-15, hIL-6, and hIL-21. 7 days later, more peptide-pulsed dendritic cells were added to each co-culture. The following day, fresh hIL2 and IL7 were added. 7 days post the final dendritic cell culture, T cells were collected and stained with an HLA-A*11:01 tetramer containing the parental epitope sequence (FIG. 16A). Flow cytometry was used to determine T cell expansion (FIG. 16B). KRAS G12D-HLA-A*11:01 tetramer staining of ATVGADGVGK (SEQ ID NO: 244) expanded T cells 7 days after 3^{rd} peptide boost showed increased population of CD3+ KRAS G12D tetramer+ T cells, indicating increased binding affinity. No tetramer control used as negative control.

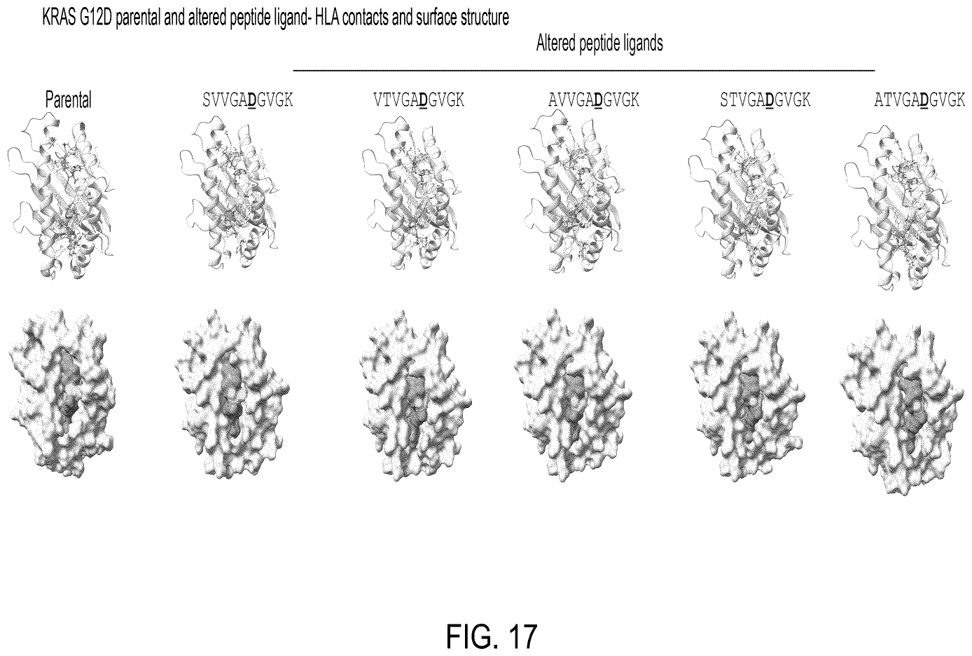

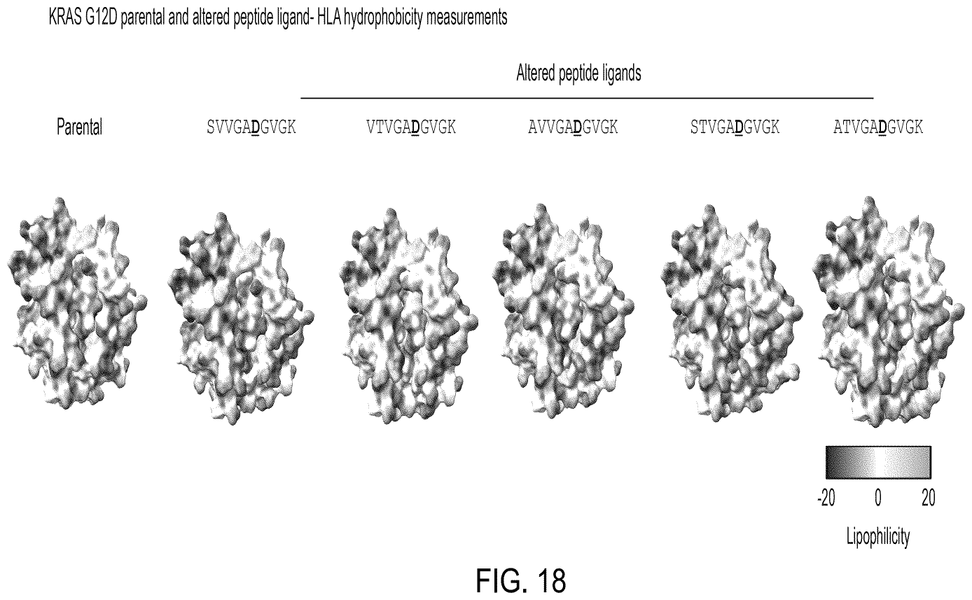

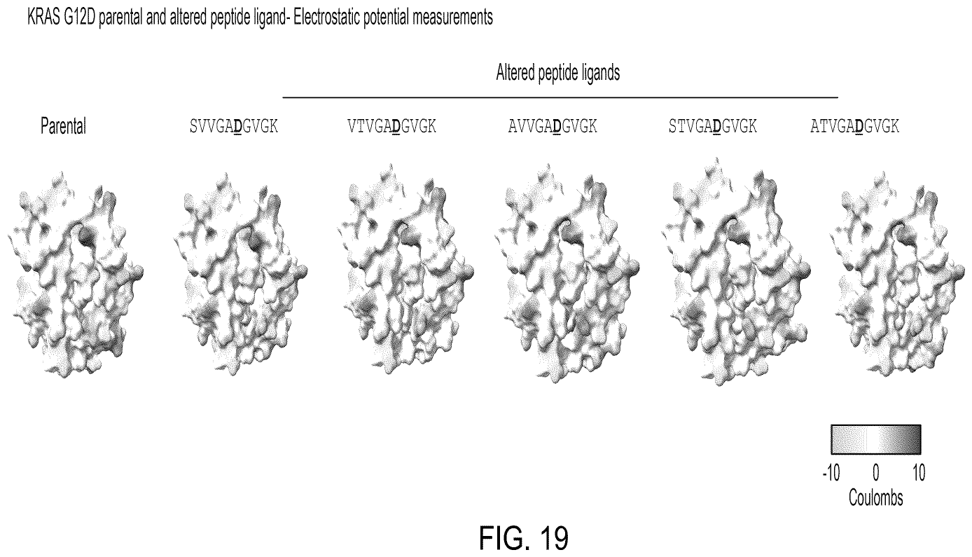

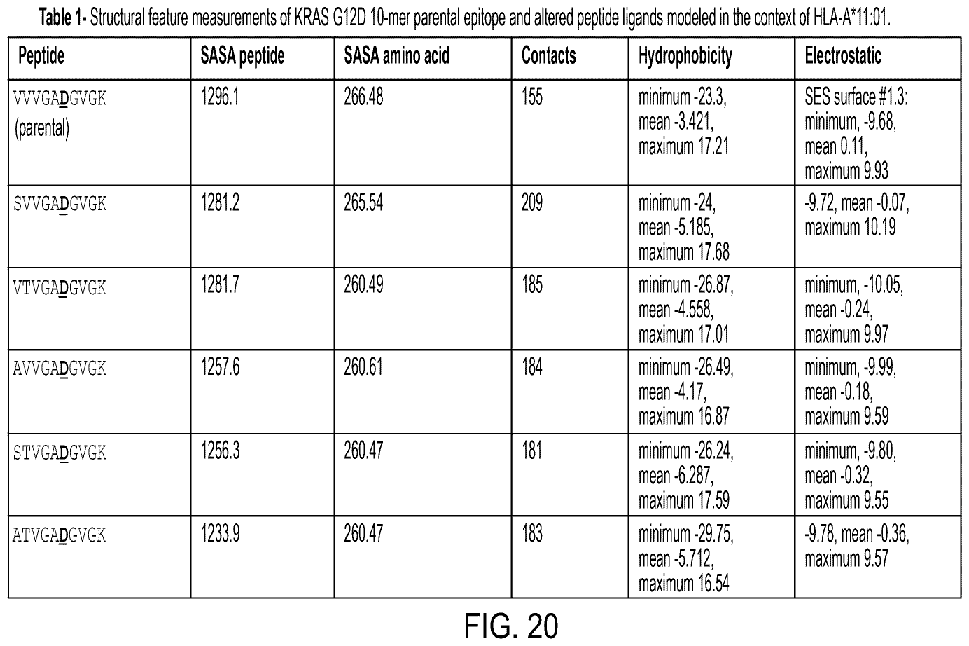

KRAS G12D parental and altered peptide ligand-HLA contacts and surface structure was determined (FIG. 17). Hydrophobicity of KRAS G12D parental and altered peptide ligand-HLA was measured (FIG. 18). Electrostatic potential of KRAS G12D parental and altered peptide ligand was measured. Structural feature measurements of KRAS G12D 10-mer parental epitope and altered peptide ligands modeled in the context of HLA-A*11:01 are summarized in FIG. 20.

### Altered Peptide Ligands (APL)

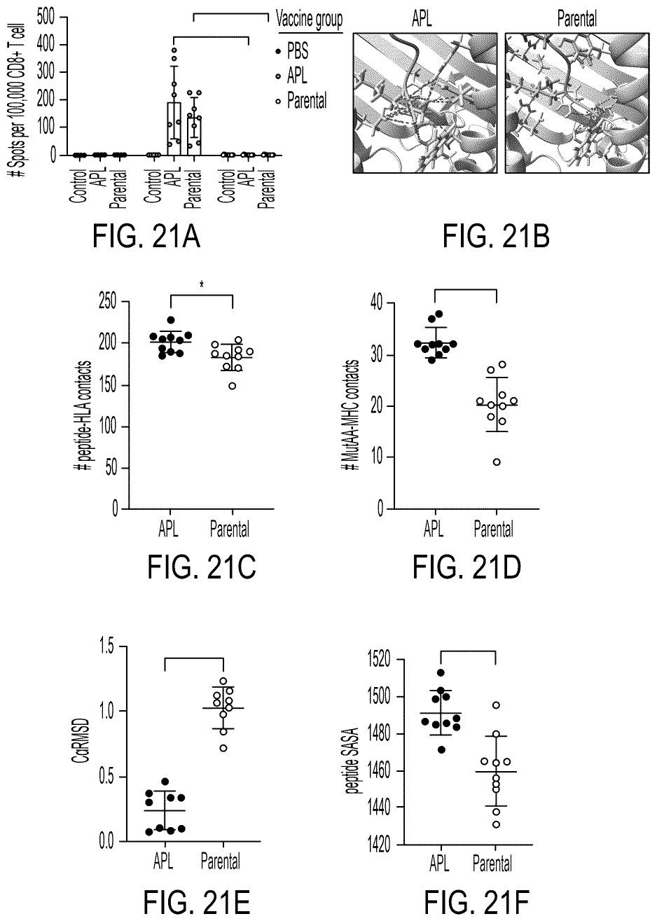

Using a Panc02 murine model, altered peptide ligands (APL) were engineered or included in a murine neoantigen-targeted vaccine (H. L. Kinkead et al., JCI Insight. 2018; 3(20):e122857. Doi.org/10.1172/jci.insight.122857). FIG. 21A-FIG. 21F demonstrate that engineered peptides display enhanced contact points and solvent accessible surface area, and reduced rigidity in a murine PDAC model. Mice were vaccinated twice 7 days apart with PBS, 50 μg APL, or 5 μg parental peptide. Seven days after the last vaccine dose, splenocytes were restimulated with APL or parental peptides, overnight, and IFNγ production was measured by ELISpot. Two-way ANOVA followed by Tukey's multiple comparisons test was performed (FIG. 21A). In silico structural modeling of APL or parental peptide in murine H2-Kb (tan, murine MHC class I molecule H2-Kb, blue, minimal epitope of peptide 44, orange, parental or engineered amino acid residue). The number of contacts between MHC binding cleft and peptide (FIG. 21C) or mutated residue (FIG. 21D) are shown. FIG. 21E shows Ca-RMSD, measure of peptide rigidity. FIG. 21F shows the solvent accessible surface area (SASA). Unpaired students t test was performed. (ns=P>0.05, *P≤0.05, **P≤0.01, ***P≤0.001, ****P≤0.0001).

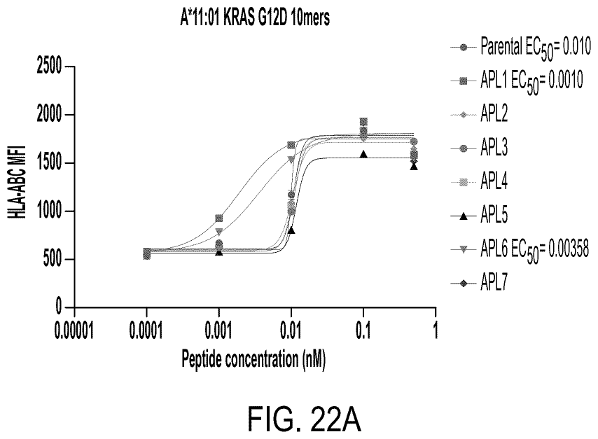

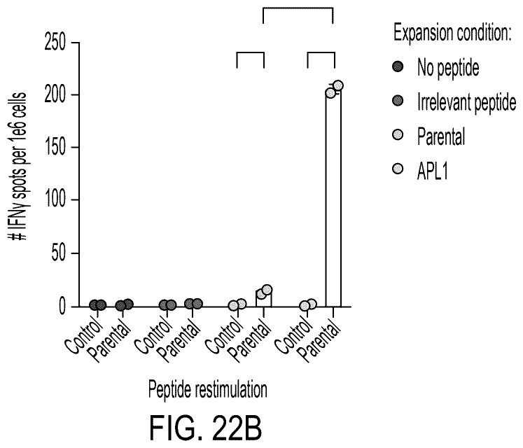

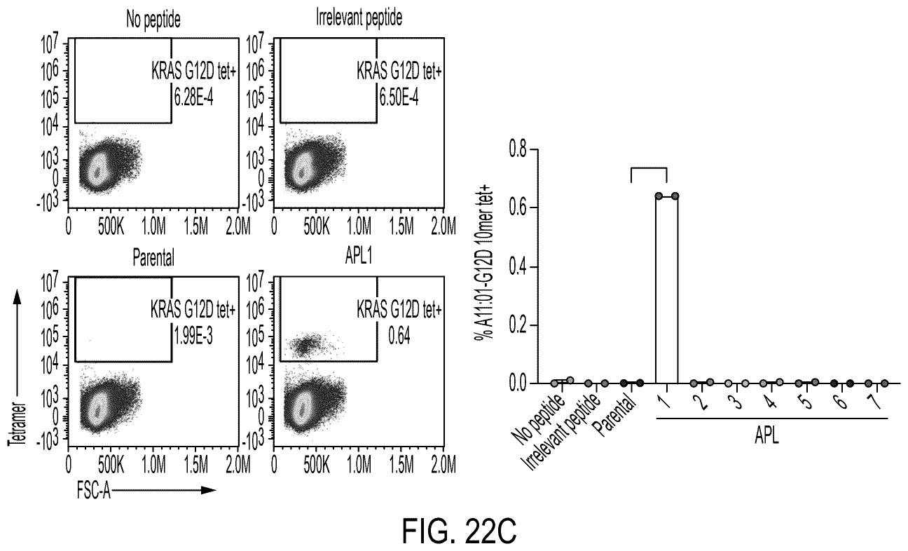

Results shown in FIGS. 22A-FIG. 22C demonstrate that KRAS G12D APL1-HLA*11:01 binding and immunogenicity of engineered antigens show superior binding and T Cell Responses. FIG. 22A shows non-linear regression curves and calculated EC50s for binding between HLA-A*11:01 and a parental KRAS G12D 10-mer peptide and 7 engineered antigens. FIG. 21B: 1×10⁶ expanded CD8 T cells from no peptide, irrelevant peptide, parental peptide, or APL1 were seeded in an ELISpot capture plate coated with anti-human IFNγ antibody. T cells were stimulated with control or parental antigen overnight and IFNγ capture was read out. Two-way ANOVA followed by uncorrected Fischer's LSD test was performed. (ns=P>0.05, *P≤0.05, **P≤0.01, ***P≤0.001, ****P≤0.0001). CD8 T cells expanded with APL 1 showed increased IFNγ spots as compared to the parental control, indicating superior T cell response. FIG. 21C shows in vitro immunogenicity testing of HLA-A*11:01 parental epitope and its engineered counterpart (APL1). Human monocyte-derived DCs (moDCs) differentiated from healthy donor PBMCs were pulsed with 100 μM parental or APL1 plus 02-microglobulin (4 hours) at 37 °C. After peptide pulsing, moDCs were washed with PBS and co-cultured with 6×10⁶ autologous CD8 T cells (plus IL-2, IL-7, IL-15, and IL-21) (7 days). Boosts were performed with peptide-pulsed moDCs on day 7, 14, and 21. T cells were collected and stained with HLA-A*11:01 tetramer loaded with parental peptide or were subject to IFNγ ELISpot.

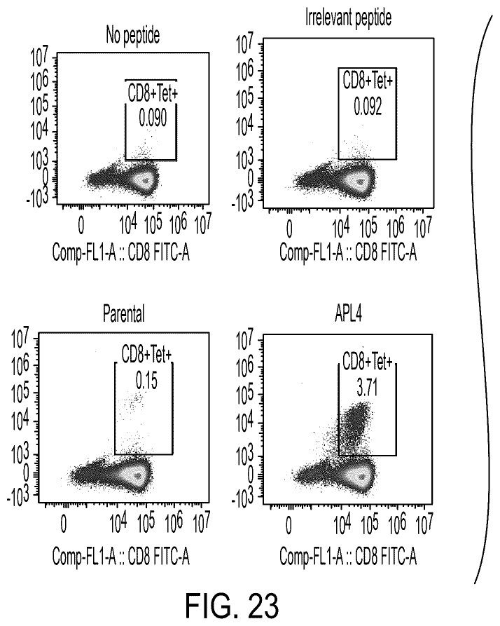

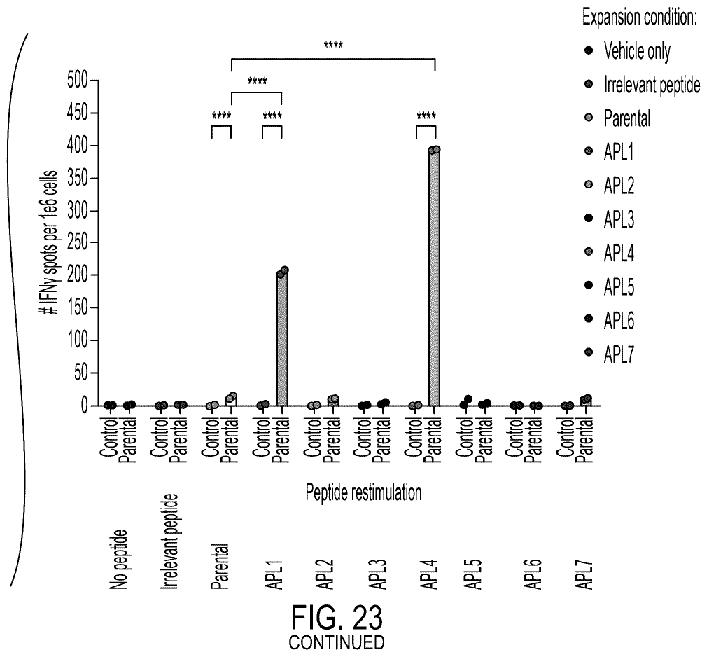

KRAS G12D APL4 demonstrates greater T cell responses compared to parental epitope in a different HLA*11:01 human donor (FIG. 23). In vitro immunogenicity testing of HLA-A*11:01 parental epitope and its engineered counterpart (APL4). Human monocyte-derived DCs (moDCs) differentiated from healthy donor PBMCs were pulsed with 100 μM parental or APL4 plus 02-microglobulin (4 hours) at 37 °C. After peptide pulsing, moDCs were washed with PBS and co-cultured with 6×10⁶ autologous CD8 T cells (plus IL-2, IL-7, IL-15, and IL-21) (7 days). Boosts were performed with peptide-pulsed moDCs on day 7, 14, and 21. T cells were collected and stained with HLA-A*11:01 tetramer loaded with parental peptide (Left) or subject to IFNγ ELISPOT (Right). APL1 and APL4-pulsed T cells showed increased IFNγ spots as compared to the parental control, indicating superior T cell response. The in vitro results validated that the structural modeling method described herein was able to predict the KRAS APLs with superior binding affinity to HLA-A*11:01.

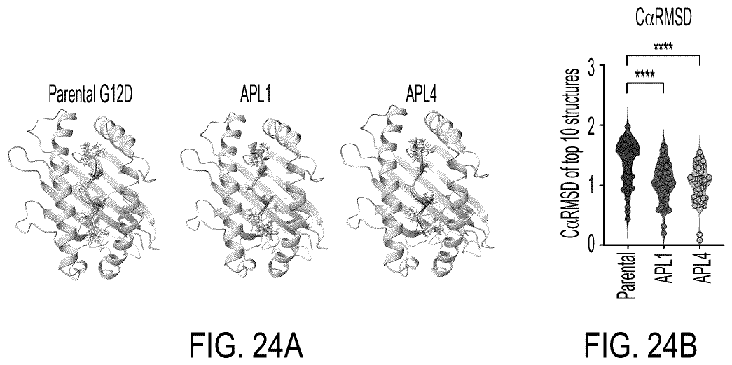

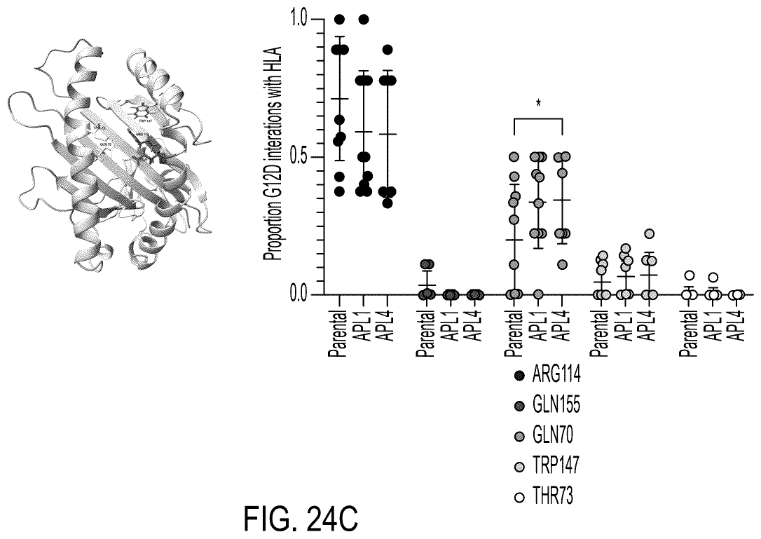

Structural analysis of KRAS G12D HLA-A*11:01 APL1 and APL4 identify peptide rigidity and stabilizing HLA cleft interactions as features associated with enhanced peptide immunogenicity (FIG. 24A-FIG. 24C). FIG. 24A_shows structural models of A11-KRAS G12D parental epitope (left), All-KRAS G12D APL1, and All-KRAS G12D APL4 in the context of HLA-A*11:01. Top 10 energetically favorable models of the peptide are shown for each peptide (n=10). FIG. 24B CaRMSD was calculated for the 10-mer peptide in each model compared to all other models for that epitope. Two way ANOVA was performed followed by Tukey's multiple comparisons test. FIG. 24C shows structural analysis of peptide neoantigen residue (G12D) in contact with HLA cleft residues that are associated with stabilization of peptide-HLA interactions (from left to right, Arg114, Gln155, Gln70, TRP147, Thr73). Quantification of G12D residue interactions with each of the HLA-cleft residues across the top 10 structural models for parental G12D, APL1, or APL4 demonstrates APL4 has an altered pattern of HLA cleft binding with a significantly higher proportion of interactions with the Gln70 residue suggested more diverse stabilizing interactions within the HLA cleft.

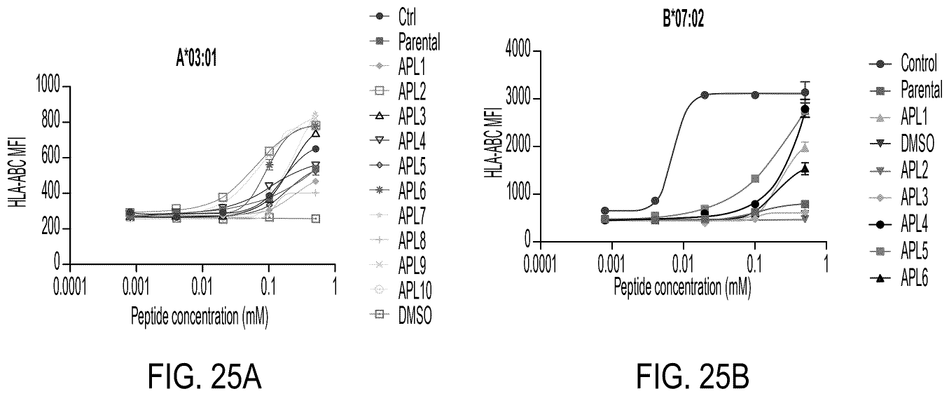

Binding affinity changes for KRAS G12D APLs in the context of HLA-A*03:01 and HLA-B*07:02 (FIG. 25A and FIG. 25B). Optimized APLs for HLA-A*03:01 and B*07:02 were design and tested for enhanced binding affinity relative to parental neoantigen in K562-TAP1_{KO}-HLA expressing cell lines. Cells were incubated with increasing concentration of peptide overnight at 37 C. Cells were then stained for HLA-ABC expression on the surface of cells. Median florescent intensity of HLA expression was quantified for each peptide at each peptide concentration in duplicates. Control peptide corresponds to a positive control peptide specific to each HLA. Non-linear regression curves were fit to each graph. The peptide sequences that correspond to APL1 and parental are summarized in the tables below. The altered residue is indicated in italics.

TABLE 1
Peptide sequences that correspond to APLI,
APL4 and parental sequence G12D (10-mer)

| | G12D (10-mer) | G12D-APL long peptide |
|---|---|---|
| Parental | VVVGADGVGK | YKLVVVGADGVGKSALTIQLI |
| | (SEQ ID NO: 1) | (SEQ ID NO: 832) |
| APL 1 | SVVGADGVGK | YKLSVVGADGVGKSALTIQLI |
| | (SEQ ID NO: 2) | (SEQ ID NO: 833) |
| APL 4 | AVVGADGVGK | YKLAVVGADGVGKSALTIQLI |
| | (SEQ ID NO: 3) | (SEQ ID NO: 834) |

TABLE 2
Peptide sequences that correspond to APL and parental sequence (HLA-A*02:01)

| | HLA-A*02:01 | | | | | | | | | |
|---|---|---|---|---|---|---|---|---|---|---|
| | G12V | G12V | G12R | G12R | G12D(10 | G12D(9 | G12A | G12A | G12C | G12C |
| | (10-mer) | (9-mer) | (10-mer) | (9-mer) | -mer) | -mer) | (10-mer) | (9-mer) | (10-mer) | (9-mer) |
| Pa- | KLVVV | LVVVG | KLVVV | LVVVG | KLVVV | LVVVG | KLVVV | LVVVG | KLVVV | LVVVG |
| ren- | GAVGV | AVGV | GARGV | ARGV | GADGV | ADGV | GAAGV | AAGV | GACGV | ACGV |
| tal | (SEQ ID | (SEQ ID | (SEQ ID | (SEQ ID | (SEQ ID | (SEQ ID | (SEQ ID | (SEQ ID | (SEQ ID | (SEQ |
| | NO: 4) | NO: 13) | NO: 22) | NO: 31) | NO: 40) | NO: 49) | NO: 58) | NO: 67) | NO: 76) | ID NO: |
| | | | | | | | | | | 85) |
| APL | KLVVV | LVVEG | KLVVV | LVVEG | KLVVV | LVVEG | KLVVV | LVVEG | KLVVV | LVVEG |
| | GAVGV | AVGV | GARGV | ARGV | GADGV | ADGV | GAAGV | AAGV | GACGV | ACGV |
| | (SEQ ID | (SEQ ID | (SEQ ID | (SEQ ID | (SEQ ID | (SEQ ID | (SEQ ID | (SEQ ID | (SEQ ID | (SEQ |
| | NO: 5) | NO: 14) | NO: 23) | NO: 32) | NO: 41) | NO: 50) | NO: 59) | NO: 68) | NO: 77) | ID NO: |
| | | | | | | | | | | 86) |
| | KLVEV | LVVDG | KLVEV | LVVDG | KLVEV | LVVDG | KLVEV | LVVDG | KLVEV | LVVDG |
| | GAVGV | AVGV | GARGV | ARGV | GADGV | ADGV | GAAGV | AAGV | GACGV | ACGV |
| | (SEQ ID | (SEQ ID | (SEQ ID | (SEQ ID | (SEQ ID | (SEQ ID | (SEQ ID | (SEQ ID | (SEQ ID | (SEQ |
| | NO: 6) | NO: 15) | NO: 24) | NO: 33) | NO: 42) | NO: 51) | NO: 60) | NO: 69) | NO: 78) | ID NO: |
| | | | | | | | | | | 87) |
| | KLVDV | LLVVG | KLVDV | LLVVG | KLVDV | LLVVG | KLVDV | LLVVG | KLVDV | LLVVG |
| | GAVGV | AVGV | GARGV | ARGV | GADGV | ADGV | GAAGV | AAGV | GACGV | ACGV |
| | (SEQ ID | (SEQ ID | (SEQ ID | (SEQ ID | (SEQ ID | (SEQ ID | (SEQ ID | (SEQ ID | (SEQ ID | (SEQ |
| | NO: 7) | NO: 16) | NO: 25) | NO: 34) | NO: 43) | NO: 52) | NO: 61) | NO: 70) | NO: 79) | ID NO: |
| | | | | | | | | | | 88) |
| | FLVVV | LLVEG | FLVVV | LLVEG | FLVVV | LLVEG | FLVVV | LLVEG | FLVVV | LLVEG |
| | GAVGV | AVGV | GARGV | ARGV | GADGV | ADGV | GAAGV | AAGV | GACGV | ACGV |
| | (SEQ ID | (SEQ ID | (SEQ ID | (SEQ ID | (SEQ ID | (SEQ ID | (SEQ ID | (SEQ ID | (SEQ ID | (SEQ |
| | NO: 8) | NO: 17) | NO: 26) | NO: 35) | NO: 44) | NO: 53) | NO: 62) | NO: 71) | NO: 80) | ID NO: |
| | | | | | | | | | | 89) |
| | KLVVV | LLVDG | KLVVV | LLVDG | KLVVV | LLVDG | KLVVV | LLVDG | KLVVV | LLVDG |
| | GAVGL | AVGV | GARGL | ARGV | GADGL | ADGV | GAAGL | AAGV | GACGL | ACGV |
| | (SEQ ID | (SEQ ID | (SEQ ID | (SEQ ID | (SEQ ID | (SEQ ID | (SEQ ID | (SEQ ID | (SEQ ID | (SEQ |
| | NO: 9) | NO: 18) | NO: 27) | NO: 36) | NO: 45) | NO: 54) | NO: 63) | NO: 72) | NO: 81) | ID NO: |
| | | | | | | | | | | 90) |
| | FLVVV | LLVVG | FLVVV | LLVVG | FLVVV | LLVVG | FLVVV | LLVVG | FLVVV | LLVVG |
| | GAVGL | AVGL | GARGL | ARGL | GADGL | ADGL | GAAGL | AAGL | GACGL | ACGL |
| | (SEQ ID | (SEQ ID | (SEQ ID | (SEQ ID | (SEQ ID | (SEQ ID | (SEQ ID | (SEQ ID | (SEQ ID | (SEQ |
| | NO: 10) | NO: 19) | NO: 28) | NO: 37) | NO: 46) | NO: 55) | NO: 64) | NO: 73) | NO: 82) | ID NO: |
| | | | | | | | | | | 91) |
| | KLVEV | LVVEG | KLVEV | LVVEG | KLVEV | LVVEG | KLVEV | LVVEG | KLVEV | LVVEG |
| | GAVGL | AVGL | GARGL | ARGL | GADGL | ADGL | GAAGL | AAGL | GACGL | ACGL |
| | (SEQ ID | (SEQ ID | (SEQ ID | (SEQ ID | (SEQ ID | (SEQ ID | (SEQ ID | (SEQ ID | (SEQ ID | (SEQ ID |
| | NO: 11) | NO: 20) | NO: 29) | NO: 38) | NO: 47) | NO: 56) | NO: 65) | NO: 74) | NO: 83) | NO: |
| | | | | | | | | | | 92) |
| | KLVDV | LVVDG | KLVDV | LVVDG | KLVDV | LVVDG | KLVDV | LVVDG | KLVDV | LVVDG |
| | GAVGL | AVGL | GARGL | ARGL | GADGL | ADGL | GAAGL | AAGL | GACGL | ACGL |
| | (SEQ ID | (SEQ ID | (SEQ ID | (SEQ ID | (SEQ ID | (SEQ ID | (SEQ ID | (SEQ ID | (SEQ ID | (SEQ ID |
| | NO: 12) | NO: 21) | NO: 30) | NO: 39) | NO: 48) | NO: 57) | NO: 66) | NO: 75) | NO: 84) | NO: |
| | | | | | | | | | | 93) |
| | G12D | G12D | G12R | G12R | G12V | G12V | G12A | G12A | G12C | G12C |
| | (10-mer) | (9-mer) | (10-mer) | (9-mer) | (10-mer) | (9-mer) | (10-mer) | (9-mer) | (10-mer) | (9-mer) |
| Pa- | VGADG | VGAD | VGARG | VGAR | VGAVG | VGAV | VGAAG | VGAA | VGACG | VGAC |
| ren- | VGKSA | GVGKS | VGKSA | GVGKS | VGKSA | GVGKS | VGKSA | GVGKS | VGKSA | GVGKS |
| tal | (SEQ ID | (SEQ ID | (SEQ ID | SEQ ID | (SEQ ID | (SEQ ID | (SEQ ID | (SEQ ID | (SEQ ID | (SEQ |
| | NO: 94) | NO: | NO: 106) | NO: | NO: 118) | NO: | NO: 130) | NO: | NO: 142) | ID NO: |
| | | 100) | | 112) | | 124) | | 136) | | 147) |
| APL | VLADG | VLADG | VLARG | VLARG | VLAVG | VLAVG | VLAAG | VLAAG | VLACG | VLACG |
| | VGKSA | VGKS | VGKSA | VGKS | VGKSA | VGKS | VGKSA | VGKS | VGKSA | VGKS |
| | (SEQ ID | (SEQ ID | (SEQ ID | (SEQ ID | (SEQ ID | (SEQ ID | (SEQ ID | (SEQ ID | (SEQ ID | (SEQ |
| | NO: 95) | NO: | NO: 107) | NO: | NO: 119) | NO: | NO: 131) | NO: | NO: 143) | ID NO: |
| | | 101) | | 113) | | 125) | | 137) | | 148) |
| | VGADG | VGAD | VGARG | VGAR | VGAVG | VGAV | VGAAG | VGAA | VGACG | VGAC |
| | VGKSL | GVGKL | VGKSL | GVGKL | VGKSL | GVGKL | VGKSL | GVGKL | VGKSL | GVGKL |
| | (SEQ ID | (SEQ ID | (SEQ ID | (SEQ ID | (SEQ ID | (SEQ ID | (SEQ ID | (SEQ ID | (SEQ ID | (SEQ |
| | NO: 96) | NO: | NO: 108) | NO: | NO: 120) | NO: | NO: 132) | NO: | NO: 434) | ID NO: |
| | | 102) | | 114) | | 126) | | 138) | | 149) |
| | VGADG | VGAD | VGARG | VGAR | VGAVG | VGAV | VGAAG | VGAA | VGACG | VGAC |
| | VGKSV | GVGK | VGKSV | GVGK | VGKSV | GVGK | VGKSV | GVGK | VGKSV | GVGK |
| | (SEQ ID | V | (SEQ ID | V | (SEQ ID | V | (SEQ ID | V | (SEQ ID | V |
| | NO: 97) | (SEQ ID | NO: 109) | (SEQ ID | NO: 121) | (SEQ ID | NO: 133) | (SEQ ID | NO: 144) | (SEQ |
| | | NO: | | NO: | | NO: | | NO: | | ID NO: |
| | | 103) | | 115) | | 127) | | 139) | | 150) |
| | VLADG | VLADG | VLARG | VLARG | VLAVG | VLAVG | VLAAG | VLAAG | VLACG | VLACG |
| | VGKSL | VGKL | VGKSL | VGKL | VGKSL | VGKL | VGKSL | VGKL | VGKSL | VGKL |
| | (SEQ ID | (SEQ ID | (SEQ ID | (SEQ ID | (SEQ ID | (SEQ ID | (SEQ ID | SEQ ID | (SEQ ID | (SEQ |
| | NO: 98) | NO: | NO: 110) | NO: | NO: 122) | NO: | NO: 134) | NO: | NO: 145) | ID NO: |
| | | 104) | | 116) | | 128) | | 140) | | 151) |
| | VLADG | VLADG | VLARG | VLARG | VLAVG | VLAVG | VLAAG | VLAAG | VLACG | VLACG |
| | VGKSV | VGKL | VGKSV | VGKL | VGKSV | VGKL | VGKSV | VGKL | VGKSV | VGKL |
| | (SEQ ID | (SEQ ID | (SEQ ID | (SEQ ID | (SEQ ID | (SEQ ID | (SEQ ID | (SEQ ID | (SEQ ID | (SEQ |
| | NO: 99) | NO: | NO: 111) | NO: | NO: 123) | NO: | NO: 135) | NO: | NO: 146) | ID NO: |
| | | 105) | | 117) | | 129) | | 141) | | 152) |

TABLE 3
Peptide sequences that correspond to APL and parental sequence (HLA-A*03:01)

| | HLA-A*03:01 | | | | | | | | | |
|---|---|---|---|---|---|---|---|---|---|---|
| | G12D | G12R | G12V | G12V | G12C | G12C | G12D | G12D | G12A | G12A |
| | (10-mer) | (9-mer) | (10-mer) | (9-mer) | (10-mer) | (9-mer) | (10-mer) | (9-mer) | (10-mer) | (9-mer) |
| Pa- | VVVGA | VVGA | VVVGA | VVGA | VVVGA | VVGA | VVVGA | VVGA | VVVGA | VVGA |
| ren- | DGVGK | RGVG | VGVGK | VGVG | CGVGK | CGVG | RGVGK | DGVG | AGVGK | AGVG |
| tal | (SEQ ID | K | (SEQ ID | K | (SEQ ID | K | (SEQ ID | K | (SEQ ID | K |
| | NO: 153) | (SEQ | NO: 169) | (SEQ | NO: 186) | (SEQ | NO: 203) | (SEQ | NO: 220) | (SEQ |
| | | ID NO: | | ID NO: | | ID NO: | | ID NO: | | ID NO: |
| | | 163) | | 180) | | 197) | | 214) | | 231) |
| APL | VLVGA | VLGAR | VLVGA | VLGAV | VLVGA | VLGAC | VLVGA | VLGAD | VLVGA | VLGAA |
| | DGVGK | GVGK | VGVGK | GVGK | CGVGK | GVGK | RGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 154) | ID NO: | NO: 170) | ID NO: | NO: 187) | ID NO: | NO: 204) | ID NO: | NO: 221) | ID NO: |
| | | 164) | | 181) | | 198) | | 215) | | 232) |
| | RVVGA | RVGAR | RVVGA | RVGAV | RVVGA | RVGAC | RVVGA | RVGAD | RVVGA | RVGAA |
| | DGVGK | GVGK | VGVGK | GVGK | CGVGK | GVGK | RGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 155) | ID NO: | NO: 171) | ID NO: | NO: 188) | ID NO: | NO: 205) | ID NO: | NO: 222) | ID NO: |
| | | 165) | | 182) | | 199) | | 216) | | 233) |
| | RLVGA | VVGA | RLVGA | VVGA | RLVGA | VVGA | RLVGA | VVGA | RLVGA | VVGA |
| | DGVGK | RGLGK | VGVGK | VGLGK | CGVGK | CGLGK | RGVGK | DGLGK | AGVGK | AGLGK |
| | (SEQ ID | (SEQ | (SEQ ID | (SEQ | (SEQ ID | (SEQ | (SEQ ID | (SEQ | (SEQ ID | (SEQ |
| | NO: 156) | ID NO: | NO: 172) | ID NO: | NO: 189) | ID NO: | NO: 206) | ID NO: | NO: 223) | ID NO: |
| | | 166) | | 183) | | 200) | | 217) | | 234) |
| | VVVGA | VLGAR | VVVGA | VLGAV | VVVGA | VLGAC | VVVGA | VLGAD | VVVGA | VLGAA |
| | DLVGK | GLGK | VLVGK | GLGK | CLVGK | GLGK | RLVGK | GLGK | ALVGK | GLGK |
| | (SEQ ID | (SEQ | (SEQ ID | (SEQ | (SEQ ID | (SEQ | (SEQ ID | (SEQ | (SEQ ID | (SEQ |
| | NO: 157) | ID NO: | NO: 173) | ID NO: | NO: 190) | ID NO: | NO: 207) | ID NO: | NO: 224) | ID NO: |
| | | 167) | | 184) | | 201) | | 218) | | 235) |
| | VLVGA | RLGAR | VLVGA | RLGAV | VLVGA | RLGAC | VLVGA | RLGAD | VLVGA | RLGAA |
| | DLVGK | GLGK | VLVGK | GLGK | CLVGK | GLGK | RLVGK | GLGK | ALVGK | GLGK |
| | (SEQ ID | (SEQ | (SEQ ID | (SEQ | (SEQ ID | (SEQ | (SEQ ID | (SEQ | (SEQ ID | (SEQ |
| | NO: 158) | ID NO: | NO: 174) | ID NO: | NO: 191) | ID NO: | NO: 208) | ID NO: | NO: 225) | ID NO: |
| | | 168) | | 185) | | 202) | | 219) | | 236) |
| | RVVGA | | RVVGA | | RVVGA | | RVVGA | | RVVGA | |
| | DLVGK | | VLVGK | | CLVGK | | RLVGK | | ALVGK | |
| | (SEQ ID | | (SEQ ID | | (SEQ ID | | (SEQ ID | | (SEQ ID | |
| | NO: 159) | | NO: 175) | | NO: 192) | | NO: 209) | | NO: 226) | |
| | RLVGA | | RLVGA | | RLVGA | | RLVGA | | RLVGA | |
| | DLVGK | | VLVGK | | CLVGK | | RLVGK | | ALVGK | |
| | (SEQ ID | | (SEQ ID | | (SEQ ID | | (SEQ ID | | (SEQ ID | |
| | NO: 160) | | NO: 176) | | NO: 193) | | NO: 210) | | NO: 227) | |
| | VLVGA | | VLVGA | | VLVGA | | VLVGA | | VLVGA | |
| | DGVGK | | VGVGK | | CGVGK | | RGVGK | | AGVGK | |
| | (SEQ ID | | (SEQ ID | | (SEQ ID | | (SEQ ID | | (SEQ ID | |
| | NO: 161) | | NO: 177) | | NO: 194) | | NO: 211) | | NO: 228) | |
| | KVVGA | | KVVGA | | KVVGA | | KVVGA | | KVVGA | |
| | DGVGK | | VGVGK | | CGVGK | | RGVGK | | AGVGK | |
| | (SEQ ID | | (SEQ ID | | (SEQ ID | | (SEQ ID | | (SEQ ID | |
| | NO: 471) | | NO: 178) | | NO: 195) | | NO: 212) | | NO: 229) | |
| | KLVGA | | KLVGA | | KLVGA | | KLVGA | | KLVGA | |
| | DGVGK | | VGVGK | | CGVGK | | RGVGK | | AGVGK | |
| | (SEQ ID | | (SEQ ID | | (SEQ ID | | (SEQ ID | | (SEQ ID | |
| | NO: 162) | | NO: 179) | | NO: 196) | | NO: 213) | | NO: 230) | |

TABLE 4
Peptide sequences that correspond to APL and parental sequence (HLA-A*11:01)

| | HLA-A*11:01 | | | | | | | | | |
|---|---|---|---|---|---|---|---|---|---|---|
| | G12D | G12D | G12R | G12R | G12V | G12V | G12C | G12C | G12A | G12A |
| | (10-mer) | (9-mer) | (10-mer) | (9-mer) | (10-mer) | (9-mer) | (10-mer) | (9-mer) | (10-mer) | (9-mer) |
| Pa- | VVVGA | VVGA | VVVGA | VVGA | VVVGA | VVGA | VVVGA | VVGA | VVVGA | VVGA |
| ren- | DGVGK | DGVG | RGVGK | RGVG | VGVGK | VGVG | CGVGK | CGVG | AGVGK | AGVG |
| tal | (SEQ ID | K | (SEQ ID | K | (SEQ ID | K | (SEQ ID | K | (SEQ ID | K |
| | NO: 237) | (SEQ | NO: 253) | (SEQ | NO: 269) | (SEQ | NO: 285) | (SEQ | NO: 301) | (SEQ |
| | | ID NO: | | ID NO: | | ID NO: | | ID NO: | | ID NO: |
| | | 245) | | 261) | | 277) | | 293) | | 309) |
| APL | SVVGA | SVGAD | SVVGA | SVGAR | SVVGA | SVGAV | SVVGA | SVGAC | SVVGA | SVGAA |
| | DGVGK | GVGK | RGVGK | GVGK | VGVGK | GVGK | CGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 238) | ID NO: | NO: 254) | ID NO: | NO: 270) | ID NO: | NO: 286) | ID NO: | NO: 302) | ID NO: |
| | | 246) | | 262) | | 278) | | 294) | | 310) |
| | VTVGA | VTGAD | VTVGA | VTGAR | VTVGA | VTGAV | VTVGA | VTGAC | VTVGA | VTGAA |
| | DGVGK | GVGK | RGVGK | GVGK | VGVGK | GVGK | CGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 239) | ID NO: | NO: 255) | ID NO: | NO: 271) | ID NO: | NO: 287) | ID NO: | NO: 303) | ID NO: |
| | | 247) | | 263) | | 279) | | 295) | | 311) |
| | VSVGA | AVGAD | VSVGA | AVGAR | VSVGA | AVGAV | VSVGA | AVGAC | VSVGA | AVGAA |
| | DGVGK | GVGK | RGVGK | GVGK | VGVGK | GVGK | CGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 240) | ID NO: | NO: 256) | ID NO: | NO: 272) | ID NO: | NO: 288) | ID NO: | NO: 304) | ID NO: |
| | | 248) | | 264) | | 280) | | 296) | | 312) |
| | AVVGA | VVGA | AVVGA | VVGA | AVVGA | VVGA | AVVGA | VVGA | AVVGA | VVGA |
| | DGVGK | DGVG | RGVGK | RGVG | VGVGK | VGVG | CGVGK | CGVG | AGVGK | AGVG |
| | (SEQ ID | K | (SEQ ID | K | (SEQ ID | K | (SEQ ID | K | (SEQ ID | K |
| | NO: 241) | (SEQ | NO: 257) | (SEQ | NO: 273) | (SEQ | NO: 289) | (SEQ | NO: 305) | (SEQ |
| | | ID NO: | | ID NO: | | ID NO: | | ID NO: | | ID NO: |
| | | 249) | | 265) | | 281) | | 297) | | 313) |
| | VVVGA | STGAD | VVVGA | STGAR | VVVGA | STGAV | VVVGA | STGAC | VVVGA | STGAA |
| | DGLGK | GVGK | RGLGK | GVGK | VGLGK | GVGK | CGLGK | GVGK | AGLGK | GVGK |
| | (SEQ ID | (SEQ | (SEQ ID | (SEQ | (SEQ ID | (SEQ | (SEQ ID | (SEQ | (SEQ ID | (SEQ |
| | NO: 242) | ID NO: | NO: 258) | ID NO: | NO: 274) | ID NO: | NO: 290) | ID NO: | NO: 306) | ID NO: |
| | | 250) | | 266) | | 282) | | 298) | | 314) |
| | STVGA | ATGAD | STVGA | ATGAR | STVGA | ATGAV | STVGA | ATGAC | STVGA | ATGAA |
| | DGVGK | GVGK | RGVGK | GVGK | VGVGK | GVGK | CGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 243) | ID NO: | NO: 259) | ID NO: | NO: 275) | ID NO: | NO: 291) | ID NO: | NO: 307) | ID NO: |
| | | 251) | | 267) | | 283) | | 299) | | 315) |
| | ATVGA | VVGA | ATVGA | VVGA | ATVGA | VVGA | ATVGA | VVGA | ATVGA | VVGA |
| | DGVGK | DGLGK | RGVGK | RGLGK | VGVGK | VGLGK | CGVGK | CGLGK | AGVGK | AGLGK |
| | (SEQ ID | (SEQ | (SEQ ID | (SEQ | (SEQ ID | (SEQ | (SEQ ID | (SEQ | (SEQ ID | (SEQ |
| | NO: 244) | ID NO: | NO: 260) | ID NO: | NO: 276) | ID NO: | NO: 292) | ID NO: | NO: 308) | ID NO: |
| | | 252) | | 268) | | 284) | | 300) | | 316) |

TABLE 5
Peptide sequences that correspond to APL and parental sequence (HLA-A*24:02)

| | HLA-A*24:02 | | | | | | | | | |
|---|---|---|---|---|---|---|---|---|---|---|
| | G12D | G12D | G12R | G12R | G12V | G12V | G12C | G12C | G12A | G12A |
| | (10-mer) | (9-mer) | (10-mer) | (9-mer) | (10-mer) | (9-mer) | (10-mer) | (9-mer) | (10-mer) | (9-mer) |
| Pa- | VVVGA | VVGA | VVVGA | VVGA | VVVGA | VVGA | VVVGA | VVGA | VVVGA | VVGA |
| ren- | DGVGK | DGVG | RGVGK | RGVG | VGVGK | VGVG | CGVGK | CGVG | AGVGK | AGVG |
| tal | (SEQ ID | K | (SEQ ID | K | (SEQ ID | K | (SEQ ID | K | (SEQ ID | K |
| | NO: 317) | (SEQ | NO: 333) | (SEQ | NO: 349) | (SEQ | NO: 365) | (SEQ | NO: 381) | (SEQ |
| | | ID NO: | | ID NO: | | ID NO: | | ID NO: | | ID NO: |
| | | 325) | | 341) | | 357) | | 373) | | 389) |
| APL | VYVGA | VYGA | VYVGA | VYGA | VYVGA | VYGA | VYVGA | VYGA | VYVGA | VYGA |
| | DGVGK | DGVG | RGVGK | RGVG | VGVGK | VGVG | CGVGK | CGVG | AGVGK | AGVG |
| | (SEQ ID | K | (SEQ ID | K | (SEQ ID | K | (SEQ ID | K | (SEQ ID | K |
| | NO: 318) | (SEQ | NO: 334) | (SEQ | NO: 350) | (SEQ | NO: 366) | (SEQ | NO: 382) | (SEQ |
| | | ID NO: | | ID NO: | | ID NO: | | ID NO: | | ID NO: |
| | | 326) | | 342) | | 358) | | 374) | | 390) |
| | VVVGA | VVGA | VVVGA | VVGA | VVVGA | VVGA | VVVGA | VVGA | VVVGA | VVGA |
| | DGVGF | DGVGF | RGVGF | RGVGF | VGVGF | VGVGF | CGVGF | CGVGF | AGVGF | AGVGF |
| | (SEQ ID | (SEQ | (SEQ ID | (SEQ | (SEQ ID | (SEQ | (SEQ ID | (SEQ | (SEQ ID | (SEQ |
| | NO: 319) | ID NO: | NO: 335) | ID NO: | NO: 351) | ID NO: | NO: 367) | ID NO: | NO: 383) | ID NO: |
| | | 327) | | 343) | | 359) | | 375) | | 391) |
| | VVLGA | VVLAD | VVLGA | VVLAR | VVLGA | VVLAV | VVLGA | VVLAC | VVLGA | VVLAA |
| | DGVGK | GVGK | RGVGK | GVGK | VGVGK | GVGK | CGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 320) | ID NO: | NO: 336) | ID NO: | NO: 352) | ID NO: | NO: 368) | ID NO: | NO: 384) | ID NO: |
| | | 328) | | 344) | | 360) | | 376) | | 392) |
| | VFVGA | VFGAD | VFVGA | VFGAR | VFVGA | VFGAV | VFVGA | VFGAC | VFVGA | VFGAA |
| | DGVGK | GVGK | RGVGK | GVGK | VGVGK | GVGK | CGVGK | GVGK | AGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 321) | ID NO: | NO: 337) | ID NO: | NO: 353) | ID NO: | NO: 369) | ID NO: | NO: 385) | ID NO: |
| | | 329) | | 345) | | 361) | | 377) | | 393) |
| | VVVGA | VVGA | VVVGA | VVGA | VVVGA | VVGA | VVVGA | VVGA | VVVGA | VVGA |
| | DGVGL | DGVGL | RGVGL | RGVGL | VGVGL | VGVGL | CGVGL | CGVGL | AGVGL | AGVGL |
| | (SEQ ID | (SEQ | (SEQ ID | (SEQ | (SEQ ID | (SEQ | (SEQ ID | (SEQ | (SEQ ID | (SEQ |
| | NO: 322) | ID NO: | NO: 338) | ID NO: | NO: 354) | ID NO: | NO: 370) | ID NO: | NO: 386) | ID NO: |
| | | 330) | | 346) | | 362) | | 378) | | 394) |
| | VYVGA | VYGA | VYVGA | VYGA | VYVGA | VYGA | VYVGA | VYGA | VYVGA | VYGA |
| | DGVGF | DGVGF | RGVGF | RGVGF | VGVGF | VGVGF | CGVGF | CGVGF | AGVGF | AGVGF |
| | (SEQ ID | (SEQ | (SEQ ID | (SEQ | (SEQ ID | (SEQ | (SEQ ID | (SEQ | (SEQ ID | (SEQ |
| | NO: 323) | ID NO: | NO: 339) | ID NO: | NO: 355) | ID NO: | NO: 371) | ID NO: | NO: 387) | ID NO: |
| | | 331) | | 347) | | 363) | | 379) | | 395) |
| | VFVGA | VFGAD | VFVGA | VFGAR | VFVGA | VFGAV | VFVGA | VFGAC | VFVGA | VFGAA |
| | DGVGL | GVGL | RGVGL | GVGL | VGVGL | GVGL | CGVGL | GVGL | AGVGL | GVGL |
| | (SEQ ID | (SEQ | (SEQ ID | (SEQ | (SEQ ID | (SEQ | (SEQ ID | (SEQ | (SEQ ID | (SEQ |
| | NO: 324) | ID NO: | NO: 340) | ID NO: | NO: 356) | ID NO: | NO: 372) | ID NO: | NO: 388) | ID NO: |
| | | 332) | | 348) | | 364) | | 380) | | 396) |

TABLE 6
Peptide sequences that correspond to APL and parental sequence (HLA-A*24:02)

| | HLA-A*24:02 | | | | |
|---|---|---|---|---|---|
| | G12D (10-mer) | G12V (10-mer) | G12R (10-mer) | G12A (10-mer) | G12C (10-mer) |
| Paren- | VGADGVGKSA | VGAVGVGKSA | VGARGVGKSA | VGAAGVGKSA | VGACGVGKSA |
| tal | (SEQ ID NO: 397) | (SEQ ID NO: 405) | (SEQ ID NO: 413) | (SEQ ID NO: 421) | (SEQ ID NO: 429) |
| APL | VYADGVGKSA | VYAVGVGKSA | VYARGVGKSA | VYAAGVGKSA | VYACGVGKSA |
| | (SEQ ID NO: 398) | (SEQ ID NO: 406) | (SEQ ID NO: 414) | (SEQ ID NO: 422) | (SEQ ID NO: 430) |
| | VGADGVGKSF | VGAVGVGKSF | VGARGVGKSF | VGAAGVGKSF | VGACGVGKSF |
| | (SEQ ID NO: 399) | (SEQ ID NO: 407) | (SEQ ID NO: 415) | (SEQ ID NO: 423) | (SEQ ID NO: 431) |
| | VYADGVGKSF | VYAVGVGKSF | VYARGVGKSF | VYAAGVGKSF | VYACGVGKSF |
| | (SEQ ID NO: 400) | (SEQ ID NO: 408) | (SEQ ID NO: 416) | (SEQ ID NO: 424) | (SEQ ID NO: 432) |
| | VFADGVGKSA | VFAVGVGKSA | VFARGVGKSA | VFAAGVGKSA | VFACGVGKSA |
| | (SEQ ID NO: 401) | (SEQ ID NO: 409) | (SEQ ID NO: 417) | (SEQ ID NO: 425) | (SEQ ID NO: 433) |
| | VGADGVGKSL | VGAVGVGKSL | VGARGVGKSL | VGAAGVGKSL | VGACGVGKSL |
| | (SEQ ID NO: 402) | (SEQ ID NO: 410) | (SEQ ID NO: 418) | (SEQ ID NO: 426) | (SEQ ID NO: 434) |
| | VFADGVGKSL | VFAVGVGKSL | VFARGVGKSL | VFAAGVGKSL | VFACGVGKSL |
| | (SEQ ID NO: 403) | (SEQ ID NO: 411) | (SEQ ID NO: 419) | (SEQ ID NO: 427) | (SEQ ID NO: 435) |
| | VYADGVGKSL | VYAVGVGKSL | VYARGVGKSL | VYAAGVGKSL | VYACGVGKSL |
| | (SEQ ID NO: 404) | (SEQ ID NO: 412) | (SEQ ID NO: 420) | (SEQ ID NO: 428) | (SEQ ID NO: 436) |

TABLE 7
Peptide sequences that correspond to APL and parental sequence (HLA-A*30:01)

| | HLA-A*30:01 | | | | | | | | | |
|---|---|---|---|---|---|---|---|---|---|---|
| | G12R | G12R | G12V | G12V | G12D | G12D | G12A | G12A | G12C | G12C |
| | (10-mer) | (9-mer) | (10-mer) | (9-mer) | (10-mer) | (9-mer) | (10-mer) | (9-mer) | (10-mer) | (9-mer) |
| Pa- | VVVGA | VVGA | VVVGA | VVGA | VVVGA | VVGA | VVVGA | VVGA | VVVGA | VVGA |
| ren- | RGVGK | RGVG | VGVGK | VGVG | DGVGK | DGVG | AGVGK | AGVG | CGVGK | CGVG |
| tal | (SEQ ID | K | (SEQ ID | K | (SEQ ID | K | (SEQ ID | K | (SEQ ID | K |
| | NO: 437) | (SEQ | NO: 451) | (SEQ | NO: 465) | (SEQ | NO: 479) | (SEQ | NO: 493) | (SEQ |
| | | ID NO: | | ID NO: | | ID NO: | | ID NO: | | ID NO: |
| | | 444) | | 458) | | 472) | | 486) | | 500) |
| APL | RVVGA | VVKA | RVVGA | VVKA | RVVGA | VVKA | RVVGA | VVKA | RVVGA | VVKA |
| | RGVGK | RGVG | VGVGK | VGVG | DGVGK | DGVG | AGVGK | AGVG | CGVGK | CGVG |
| | (SEQ ID | K | (SEQ ID | K | (SEQ ID | K | (SEQ ID | K | (SEQ ID | K |
| | NO: 438) | (SEQ | NO: 452) | (SEQ | NO: 466) | (SEQ | NO: 480) | (SEQ | NO: 494) | (SEQ |
| | | ID NO: | | ID NO: | | ID NO: | | ID NO: | | ID NO: |
| | | 445) | | 459) | | 473) | | 487) | | 501) |
| | VVKGA | KVGA | VVKGA | KVGA | VVKGA | KVGA | VVKGA | KVGA | VVKGA | KVGA |
| | RGVGK | RGVG | VGVGK | VGVG | DGVGK | DGVG | AGVGK | AGVG | CGVGK | CGVG |
| | (SEQ ID | K | (SEQ ID | K | (SEQ ID | K | (SEQ ID | K | (SEQ ID | K |
| | NO: 439) | (SEQ | NO: 453) | (SEQ | NO: 467) | (SEQ | NO: 481) | (SEQ | NO: 495) | (SEQ |
| | | ID NO: | | ID NO: | | ID NO: | | ID NO: | | ID NO: |
| | | 446) | | 460) | | 474) | | 488) | | 502) |
| | VTVGA | VTGAR | VTVGA | VTGAV | VTVGA | VTGAD | VTVGA | VTGAA | VTVGA | VTGAC |
| | RGVGK | GVGK | VGVGK | GVGK | DGVGK | GVGK | AGVGK | GVGK | CGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 440) | ID NO: | NO: 454) | ID NO: | NO: 468) | ID NO: | NO: 482) | ID NO: | NO: 496) | ID NO: |
| | | 447) | | 461) | | 475) | | 489) | | 503) |
| | VVVGA | VVGA | VVVGA | VVGA | VVVGA | VVGA | VVVGA | VVGA | VVVGA | VVGA |
| | RGPGK | RGPGK | VGPGK | VGPGK | DGPGK | DGPGK | AGPGK | AGPGK | CGPGK | CGPGK |
| | (SEQ ID | (SEQ | (SEQ ID | (SEQ | (SEQ ID | (SEQ | (SEQ ID | (SEQ | (SEQ ID | (SEQ |
| | NO: 441) | ID NO: | NO: 455) | ID NO: | NO: 469) | ID NO: | NO: 483) | ID NO: | NO: 497) | ID NO: |
| | | 448) | | 462) | | 476) | | 490) | | 504) |
| | VVVGA | KVKA | VVVGA | KVKA | VVVGA | KVKA | VVVGA | KVKA | VVVGA | KVKA |
| | RRVGK | RGVG | VRVGK | VGVG | DRVGK | DGVG | ARVGK | AGVG | CRVGK | CGVG |
| | (SEQ ID | K | (SEQ ID | K | (SEQ ID | K | (SEQ ID | K | (SEQ ID | K |
| | NO: 442) | (SEQ | NO: 456) | (SEQ | NO: 470) | (SEQ | NO: 484) | (SEQ | NO: 498) | (SEQ |
| | | ID NO: | | ID NO: | | ID NO: | | ID NO: | | ID NO: |
| | | 449) | | 463) | | 477) | | 491) | | 505) |
| | KVVGA | KTGAR | KVVGA | KTGAV | KVVGA | KTGAD | KVVGA | KTGAA | KVVGA | KTGAC |
| | RGVGK | GVGK | VGVGK | GVGK | DGVGK | GVGK | AGVGK | GVGK | CGVGK | GVGK |
| | (SEQ ID | (SEQ | (SEQ ID | (SEQ | (SEQ ID | (SEQ | (SEQ ID | (SEQ | (SEQ ID | (SEQ |
| | NO: 443) | ID NO: | NO: 457) | ID NO: | NO: 471) | ID NO: | NO: 485) | ID NO: | NO: 499) | ID NO: |
| | | 450) | | 464) | | 478) | | 492) | | 506) |

TABLE 8
Peptide sequences that correspond to APL and parental sequence (HLA-A*68:01)

| | HLA-A*68:01 | | | | | | | | | |
|---|---|---|---|---|---|---|---|---|---|---|
| | G12C | G12V | G12D | G12R | G12V | G12A | G12R | G12D | G12A | G12C |
| | (10-mer) | (9-mer) | (10-mer) | (10-mer) | (10-mer) | (10-mer) | (9-mer) | (9-mer) | (9-mer) | (9-mer) |
| Pa- | VVVGA | VVGA | VVVGA | VVVGA | VVVGA | VVVGA | VVGA | VVGA | VVGA | VVGA |
| ren- | CGVGK | VGVG | DGVGK | RGVGK | VGVGK | AGVGK | RGVG | DGVG | AGVG | CGVG |
| tal | (SEQ ID | K | (SEQ ID | (SEQ ID | (SEQ ID | (SEQ ID | K | K | K | K |
| | NO: 507) | (SEQ | NO: 519) | NO: 525) | NO: 531) | NO: 537) | (SEQ | (SEQ | (SEQ | SEQ ID |
| | | ID NO: | | | | | ID NO: | ID NO: | ID NO: | NO: |
| | | 513) | | | | | 543) | 549) | 555) | 561) |
| APL | EVVGA | VTGAV | EVVGA | EVVGA | EVVGA | EVVGA | VTGAR | VTGAD | VTGAA | VTGAC |
| | CGVGK | GVGK | DGVGK | RGVGK | VGVGK | AGVGK | GVGK | GVGK | GVGK | GVGK |
| | (SEQ ID | (SEQ | (SEQ ID | (SEQ ID | (SEQ ID | (SEQ ID | (SEQ | (SEQ | (SEQ | SEQ ID |
| | NO: 508) | ID NO: | NO: 520) | NO: 526) | NO: 532) | NO: 538) | ID NO: | ID NO: | ID NO: | NO: |
| | | 514) | | | | | 544) | 550) | 556) | 562) |
| | VTVGA | VVGA | VTVGA | VTVGA | VTVGA | VTVGA | VVGA | VVGA | VVGA | VVGA |
| | CGVGK | VGVG | DGVGK | RGVGK | VGVGK | AGVGK | RGVG | DGVG | AGVG | CGVG |
| | (SEQ ID | R | (SEQ ID | (SEQ ID | (SEQ ID | (SEQ ID | R | R | R | R |
| | NO: 509) | (SEQ | NO: 521) | NO: 527) | NO: 533) | NO: 539) | (SEQ | (SEQ | (SEQ | SEQ ID |
| | | ID NO: | | | | | ID NO: | ID NO: | ID NO: | NO: |
| | | 515) | | | | | 545) | 551) | 557) | 563) |
| | ETVGA | VTGAV | ETVGA | ETVGA | ETVGA | ETVGA | VTGAR | VTGAD | VTGAA | VTGAC |
| | CGVGK | GVGR | DGVGK | RGVGK | VGVGK | AGVGK | GVGR | GVGR | GVGR | G |
| | (SEQ ID | (SEQ | (SEQ ID | (SEQ ID | (SEQ ID | (SEQ ID | (SEQ | (SEQ | (SEQ | VGR |
| | NO: 510) | ID NO: | NO: 522) | NO: 528) | NO: 534) | NO: 540) | ID NO: | ID NO: | ID NO: | SEQ ID |
| | | 516) | | | | | 546) | 552) | 558) | NO: |
| | | | | | | | | | | 564) |
| | VVVGA | VSGAV | VVVGA | VVVGA | VVVGA | VVVGA | VSGAR | VSGAD | VSGAA | VSGAC |
| | CGVGR | GVGK | DGVGR | RGVGR | VGVGR | AGVGR | GVGK | GVGK | GVGK | GVGK |
| | (SEQ ID | (SEQ | (SEQ ID | (SEQ ID | (SEQ ID | (SEQ ID | (SEQ | (SEQ | (SEQ | SEQ ID |
| | NO: 511) | ID NO: | NO: 523) | NO: 529) | NO: 535) | NO: 541) | ID NO: | ID NO: | ID NO: | NO: |
| | | 517) | | | | | 547) | 553) | 559) | 565) |
| | EVVGA | VLGAV | EVVGA | EVVGA | EVVGA | EVVGA | VLGAR | VLGAD | VLGAA | VLGAC |
| | CGVGR | GVGK | DGVGR | RGVGR | VGVGR | AGVGR | GVGK | GVGK | GVGK | GVGK |
| | (SEQ ID | (SEQ | (SEQ ID | (SEQ ID | (SEQ ID | (SEQ ID | (SEQ | (SEQ | (SEQ | SEQ ID |
| | NO: 512) | ID NO: | NO: 524) | NO: 530) | NO: 536) | NO: 542) | ID NO: | ID NO: | ID NO: | NO: |
| | | 518) | | | | | 548) | 554) | 560) | 566) |

TABLE 9
Peptide sequences that correspond to APL and parental sequence (HLA-B*07:02)

| | HLA-B*07:02 | | | | |
|---|---|---|---|---|---|
| | G12D (10-mer) | G12R(10-mer) | G12A (10-mer) | G12C (10-mer) | G12V (10-mer) |
| Pa- | GADGVGKSAL | GARGVGKSAL | GAAGVGKSAL | GACGVGKSAL | GAVGVGKSAL |
| ren- | (SEQ ID NO: 567) | (SEQ ID NO: 574) | (SEQ ID NO: 581) | (SEQ ID NO: 588) | (SEQ ID NO: 595) |
| tal | | | | | |
| APL | GPDGVGKSAL | GPRGVGKSAL | GPAGVGKSAL | GPCGVGKSAL | GPVGVGKSAL |
| | (SEQ ID NO: 568) | (SEQ ID NO: 575) | (SEQ ID NO: 582) | (SEQ ID NO: 589) | (SEQ ID NO: 596) |
| | AADGVGKSAL | AARGVGKSAL | AAAGVGKSAL | AACGVGKSAL | AAVGVGKSAL |
| | (SEQ ID NO: 569) | (SEQ ID NO: 576) | (SEQ ID NO: 583) | (SEQ ID NO: 590) | (SEQ ID NO: 597) |
| | SADGVGKSAL | SARGVGKSAL | SAAGVGKSAL | SACGVGKSAL | SAVGVGKSAL |
| | (SEQ ID NO: 570) | (SEQ ID NO: 577) | (SEQ ID NO: 584) | (SEQ ID NO: 591) | (SEQ ID NO: 598) |
| | APDGVGKSAL | APRGVGKSAL | APAGVGKSAL | APCGVGKSAL | APVGVGKSAL |
| | (SEQ ID NO: 571) | (SEQ ID NO: 578) | (SEQ ID NO: 585) | (SEQ ID NO: 592) | (SEQ ID NO: 599) |
| | SPDPVGKSAL | SPRPVGKSAL | SPAPVGKSAL | SPCPVGKSAL | SPVPVGKSAL |
| | (SEQ ID NO: 572) | (SEQ ID NO: 579) | (SEQ ID NO: 586) | (SEQ ID NO: 593) | (SEQ ID NO: 600) |
| | GADPVGKSAL | GARPVGKSAL | GAAPVGKSAL | GACPVGKSAL | GAVPVGKSAL |
| | (SEQ ID NO: 573) | (SEQ ID NO: 580) | (SEQ ID NO: 587) | (SEQ ID NO: 594) | (SEQ ID NO: 601) |

TABLE 10
Peptide sequences that correspond to APL and parental sequence (HLA-C*01:02)

| | HLA-C*01:02 | | | | |
|---|---|---|---|---|---|
| | G12V (9-mer) | G12R (9-mer) | G12D (9-mer) | G12A (9-mer) | G12C (9-mer) |
| Pa- | AVGVGKSAL | ARGVGKSAL | ADGVGKSAL | AAGVGKSAL | ACGVGKSAL |
| ren- | (SEQ ID NO: 602) | (SEQ ID NO: 608) | (SEQ ID NO: 614) | (SEQ ID NO: 620) | (SEQ ID NO: 626) |
| tal | | | | | |
| APL | AVPVGKSAL | ARPVGKSAL | ADPVGKSAL | AAPVGKSAL | ACPVGKSAL |
| | (SEQ ID NO: 603) | (SEQ ID NO: 609) | (SEQ ID NO: 615) | (SEQ ID NO: 621) | (SEQ ID NO: 627) |
| | AVGVGKSSL | ARGVGKSSL | ADGVGKSSL | AAGVGKSSL | ACGVGKSSL |
| | (SEQ ID NO: 604) | (SEQ ID NO: 610) | (SEQ ID NO: 616) | (SEQ ID NO: 622) | (SEQ ID NO: 628) |
| | SVGVGKSAL | SRGVGKSAL | SDGVGKSAL | SAGVGKSAL | SCGVGKSAL |
| | (SEQ ID NO: 605) | (SEQ ID NO: 611) | (SEQ ID NO: 617) | (SEQ ID NO: 623) | (SEQ ID NO: 629) |
| | AVGVGKSEL | ARGVGKSEL | ADGVGKSEL | AAGVGKSEL | ACGVGKSEL |
| | (SEQ ID NO: 606) | (SEQ ID NO: 612) | (SEQ ID NO: 618) | (SEQ ID NO: 624) | (SEQ ID NO: 630) |
| | AVAVGKSAL | ARAVGKSAL | ADAVGKSAL | AAAVGKSAL | ACAVGKSAL |
| | (SEQ ID NO: 607) | (SEQ ID NO: 613) | (SEQ ID NO: 619) | (SEQ ID NO: 625) | (SEQ ID NO: 631) |

TABLE 11
Peptide sequences that correspond to APL and parental sequence (HLA-B*08:02)

| | HLA-C*08:02 | | | | | | | | | |
|---|---|---|---|---|---|---|---|---|---|---|
| | G12D | G12D | G12V | G12V | G12R | G12R | G12A | G12A | G12C | G12C |
| | (10-mer) | (9-mer) | (10-mer) | (9-mer) | (10-mer) | (9-mer) | (10-mer) | (9-mer) | (10-mer) | (9-mer) |
| Pa- | GADGV | GADG | GAVGV | GAVG | GARGV | GARG | GAAGV | GAAG | GACGV | GACG |
| ren- | GKSAL | VGKSA | GKSAL | VGKSA | GKSAL | VGKSA | GKSAL | VGKSA | GKSAL | VGKSA |
| tal | (SEQ ID | (SEQ | (SEQ ID | (SEQ | (SEQ ID | (SEQ | (SEQ ID | (SEQ | (SEQ ID | (SEQ |
| | NO: 632) | ID NO: | NO: 645) | ID NO: | NO: 658) | ID NO: | NO: 671) | ID NO: | NO: 684) | ID NO: |
| | | 639) | | 652 | | 665) | | 678) | | 691) |
| APL | GADGV | GADG | GAVGV | GAVG | GARGV | GARG | GAAGV | GAAG | GACGV | GACG |
| | GKSAL | VGKSL | GKSAL | VGKSL | GKSAL | VGKSL | GKSAL | VGKSL | GKSAL | VGKSL |
| | (SEQ ID | (SEQ | (SEQ ID | (SEQ | (SEQ ID | (SEQ | (SEQ ID | (SEQ | (SEQ ID | (SEQ |
| | NO: 633) | ID NO: | NO: 646) | ID NO: | NO: 659) | ID NO: | NO: 672) | ID NO: | NO: 685) | ID NO: |
| | | 640) | | 653) | | 666) | | 679) | | 692) |
| | GVDGV | GADG | GVVGV | GAVG | GVRGV | GARG | GVAGV | GAAG | GVCGV | GACG |
| | GKSAL | VGKSV | GKSAL | VGKSV | GKSAL | VGKSV | GKSAL | VGKSV | GKSAL | VGKSV |
| | (SEQ ID | (SEQ | (SEQ ID | (SEQ | (SEQ ID | (SEQ | (SEQ ID | (SEQ | (SEQ ID | (SEQ |
| | NO: 634) | ID NO: | NO: 647) | ID NO: | NO: 660) | ID NO: | NO: 673) | ID NO: | NO: 686) | ID NO: |
| | | 641) | | 654) | | 667) | | 680) | | 693) |
| | VADGV | GADG | VAVGV | GAVG | VARGV | GARG | VAAGV | GAAG | VACGV | GACG |
| | GKSAL | VGKSF | GKSAL | VGKSF | GKSAL | VGKSF | GKSAL | VGKSF | GKSAL | VGKSF |
| | (SEQ ID | (SEQ | (SEQ ID | (SEQ | (SEQ ID | (SEQ | (SEQ ID | (SEQ | (SEQ ID | (SEQ |
| | NO: 635) | ID NO: | NO: 648) | ID NO: | NO: 661) | ID NO: | NO: 674) | ID NO: | NO: 687) | ID NO: |
| | | 642) | | 655) | | 668) | | 681) | | 694) |
| | GADGV | GSDGV | GAVGV | GSVGV | GARGV | GSRGV | GAAGV | GSAGV | GACGV | GSCGV |
| | GKSSL | GKSA | GKSSL | GKSA | GKSSL | GKSA | GKSSL | GKSA | GKSSL | GKSA |
| | (SEQ ID | (SEQ | (SEQ ID | (SEQ | (SEQ ID | (SEQ | (SEQ ID | (SEQ | (SEQ ID | (SEQ |
| | NO: 636) | ID NO: | NO: 649) | ID NO: | NO: 662) | ID NO: | NO: 675) | ID NO: | NO: 688) | ID NO: |
| | | 643) | | 656) | | 669) | | 682) | | 695) |
| | VADGV | VADGV | VAVGV | VAVGV | VARGV | VARGV | VAAGV | VAAGV | VACGV | VACGV |
| | GKSSL | GKSL | GKSSL | GKSL | GKSSL | GKSL | GKSSL | GKSL | GKSSL | GKSL |
| | (SEQ ID | (SEQ | (SEQ ID | (SEQ | (SEQ ID | (SEQ | (SEQ ID | (SEQ | (SEQ ID | (SEQ |
| | NO: 637) | ID NO: | NO: 650) | ID NO: | NO: 663) | ID NO: | NO: 676) | ID NO: | NO: 689) | ID NO: |
| | GADGV | 644) | GAVGV | 657) | GARGV | 670) | GAAGV | 683) | GACGV | 696) |
| | GKSEL | | GKSEL | | GKSEL | | GKSEL | | GKSEL | |
| | (SEQ ID | | (SEQ ID | | (SEQ ID | | (SEQ ID | | (SEQ ID | |
| | NO: 638) | | NO: 651) | | NO: 664) | | NO: 677) | | NO: 690) | |

TABLE 12
Peptide sequences that correspond to APL and parental sequence (HLA-C*04:01)

| | HLA-C*04:01 | | | | | | | | | |
|---|---|---|---|---|---|---|---|---|---|---|
| | G12D | G12D | G12V | G12V | G12R | G12R | G12A | G12A | G12C | G12C |
| | (10-mer) | (9-mer) | (10-mer) | (9-mer) | (10-mer) | (9-mer) | (10-mer) | (9-mer) | (10-mer) | (9-mer) |
| Pa- | GADGV | GADG | GAVGV | GAVG | GARGV | GARG | GAAGV | GAAG | GACGV | GACG |
| ren- | GKSAL | VGKSA | GKSAL | VGKSA | GKSAL | VGKSA | GKSAL | VGKSA | GKSAL | VGKSA |
| tal | (SEQ ID | (SEQ | (SEQ ID | (SEQ | (SEQ ID | (SEQ | (SEQ ID | (SEQ | (SEQ ID | (SEQ |
| | NO: 697) | ID NO: | NO: 712) | ID NO: | NO: 727) | ID NO: | NO: 742) | ID NO: | NO: 757) | ID NO: |
| | | 704) | | 719) | | 734) | | 749) | | 764) |
| APL | GFDGV | GFDGV | GFVGV | GFVGV | GFRGV | GFRGV | GFAGV | GFAGV | GFCGV | GFCGV |
| | GKSAL | GKSA | GKSAL | GKSA | GKSAL | GKSA | GKSAL | GKSA | GKSAL | GKSA |
| | (SEQ ID | (SEQ | (SEQ ID | (SEQ | (SEQ ID | (SEQ | (SEQ ID | (SEQ | (SEQ ID | (SEQ |
| | NO: 698) | ID NO: | NO: 713) | ID NO: | NO: 728) | ID NO: | NO: 743) | ID NO: | NO: 758) | ID NO: |
| | | 705) | | 720) | | 735) | | 750) | | 765) |
| | GADGV | GADG | GAVGV | GAVG | GARGV | GARG | GAAGV | GAAG | GACGV | GACG |
| | GKSAF | VGKSL | GKSAF | VGKSL | GKSAF | VGKSL | GKSAF | VGKSL | GKSAF | VGKSL |
| | (SEQ ID | (SEQ | (SEQ ID | (SEQ | (SEQ ID | (SEQ | (SEQ ID | (SEQ | (SEQ ID | (SEQ |
| | NO: 699) | ID NO: | NO: 714) | ID NO: | NO: 729) | ID NO: | NO: 744) | ID NO: | NO: 759) | ID NO: |
| | | 706) | | 721) | | 736) | | 751) | | 766) |
| | GYDGV | GYDG | GYVGV | GYVG | GYRGV | GYRG | GYAGV | GYAG | GYCGV | GYCG |
| | GKSAL | VGKSA | GKSAL | VGKSA | GKSAL | VGKSA | GKSAL | VGKSA | GKSAL | VGKSA |
| | (SEQ ID | (SEQ | (SEQ ID | (SEQ | (SEQ ID | (SEQ | (SEQ ID | (SEQ | (SEQ ID | (SEQ |
| | NO: 700) | ID NO: | NO: 715) | ID NO: | NO: 730) | ID NO: | NO: 745) | ID NO: | NO: 760) | ID NO: |
| | | 707) | | 722) | | 737) | | 752) | | 767) |
| | GFDGV | GADG | GFVGV | GAVG | GFRGV | GARG | GFAGV | GAAG | GFCGV | GACG |
| | GKSAF | VGKSF | GKSAF | VGKSF | GKSAF | VGKSF | GKSAF | VGKSF | GKSAF | VGKSF |
| | (SEQ ID | (SEQ | (SEQ ID | (SEQ | (SEQ ID | (SEQ | (SEQ ID | (SEQ | (SEQ ID | (SEQ |
| | NO: 701) | ID NO: | NO: 716) | ID NO: | NO: 731) | ID NO: | NO: 746) | ID NO: | NO: 761) | ID NO: |
| | | 708) | | 723) | | 738) | | 753) | | 768) |
| | GYDGV | GYDG | GYVGV | GYVG | GYRGV | GYRG | GYAGV | GYAG | GYCGV | GYCG |
| | GKSAF | VGKSL | GKSAF | VGKSL | GKSAF | VGKSL | GKSAF | VGKSL | GKSAF | VGKSL |
| | (SEQ ID | (SEQ | (SEQ ID | (SEQ | (SEQ ID | (SEQ | (SEQ ID | (SEQ | (SEQ ID | (SEQ |
| | NO: 702) | ID NO: | NO: 717) | ID NO: | NO: 732) | ID NO: | NO: 747) | ID NO: | NO: 762) | ID NO: |
| | | 709) | | 724) | | 739) | | 754) | | 769) |
| | VFDGV | GYDG | VFVGV | GYVG | VFRGV | GYRG | VFAGV | GYAG | VFCGV | GYCG |
| | GKSAL | VGKSF | GKSAL | VGKSF | GKSAL | VGKSF | GKSAL | VGKSF | GKSAL | VGKSF |
| | (SEQ ID | (SEQ | (SEQ ID | (SEQ | (SEQ ID | (SEQ | (SEQ ID | (SEQ | (SEQ ID | (SEQ |
| | NO: 703) | ID NO: | NO: 718) | ID NO: | NO: 733) | ID NO: | NO: 748) | ID NO: | NO: 763) | ID NO: |
| | | 710) | | 725) | | 740) | | 755) | | 770) |
| | | GFDGV | | GFVGV | | GFRGV | | GFAGV | | GFCGV |
| | | GKSL | | GKSL | | GKSL | | GKSL | | GKSL |
| | | (SEQ | | (SEQ | | (SEQ | | (SEQ | | (SEQ |
| | | ID NO: | | ID NO: | | ID NO: | | ID NO: | | ID NO: |
| | | 711) | | 726) | | 741) | | 756) | | 771) |

TABLE 13
Peptide sequences that correspond to APL and parental sequence (HLA-C*03:03)

| | HLA-C*03:03 | | | | | | | | | |
|---|---|---|---|---|---|---|---|---|---|---|
| | G12V | G12V | G12R | G12R | G12D | G12D | G12A | G12A | G12C | G12C |
| | (10-mer) | (9-mer) | (10-mer) | (9-mer) | (10-mer) | (9-mer) | (10-mer) | (9-mer) | (10-mer) | (9-mer) |
| Pa- | GAVGV | GAVG | GARGV | GARG | GADGV | GADG | GAAGV | GAAG | GACGV | GACG |
| ren- | GKSAL | VGKSA | GKSAL | VGKSA | GKSAL | VGKSA | GKSAL | VGKSA | GKSAL | VGKSA |
| tal | (SEQ ID | (SEQ | (SEQ ID | (SEQ | (SEQ ID | (SEQ | (SEQ ID | (SEQ | (SEQ ID | (SEQ |
| | NO: 772) | ID NO: | NO: 784) | ID NO: | NO: 796) | ID NO: | NO: 808) | ID NO: | NO: 820) | ID NO: |
| | | 778) | | 790) | | 802) | | 814) | | 826) |
| APL | FAVGV | FAVGV | FARGV | FARGV | FADGV | FADGV | FAAGV | FAAGV | FACGV | FACGV |
| | GKSAL | GKSA | GKSAL | GKSA | GKSAL | GKSA | GKSAL | GKSA | GKSAL | GKSA |
| | (SEQ ID | (SEQ | (SEQ ID | (SEQ | (SEQ ID | (SEQ | (SEQ ID | (SEQ | (SEQ ID | (SEQ |
| | NO: 773) | ID NO: | NO: 785) | ID NO: | NO: 797) | ID NO: | NO: 809) | ID NO: | NO: 821) | ID NO: |
| | | 779) | | 791) | | 803) | | 815) | | 827) |
| | YAVGV | YAVGV | YARGV | YARGV | YADGV | YADGV | YAAGV | YAAGV | YACGV | YACGV |
| | GKSAL | GKSA | GKSAL | GKSA | GKSAL | GKSA | GKSAL | GKSA | GKSAL | GKSA |
| | (SEQ ID | (SEQ | (SEQ ID | (SEQ | (SEQ ID | (SEQ | (SEQ ID | (SEQ | (SEQ ID | (SEQ |
| | NO: 774) | ID NO: | NO: 786) | ID NO: | NO: 798) | ID NO: | NO: 810) | ID NO: | NO: 822) | ID NO: |
| | | 780) | | 792) | | 804) | | 816) | | 828) |
| | LAVGV | LAVGV | LARGV | LARGV | LADGV | LADGV | LAAGV | LAAGV | LACGV | LACGV |
| | GKSAL | GKSA | GKSAL | GKSA | GKSAL | GKSA | GKSAL | GKSA | GKSAL | GKSA |
| | (SEQ ID | (SEQ | (SEQ ID | (SEQ | (SEQ ID | (SEQ | (SEQ ID | (SEQ | (SEQ ID | (SEQ |
| | NO: 775) | ID NO: | NO: 787) | ID NO: | NO: 799) | ID NO: | NO: 811) | ID NO: | NO: 823) | ID NO: |
| | | 781) | | 793) | | 805) | | 817) | | 829) |
| | GAVPV | GAVG | GARPV | GARG | GADPV | GADG | GAAPV | GAAG | GACPV | GACG |
| | GKSAL | VGKSL | GKSAL | VGKSL | GKSAL | VGKSL | GKSAL | VGKSL | GKSAL | VGKSL |
| | (SEQ ID | (SEQ | (SEQ ID | (SEQ | (SEQ ID | (SEQ | (SEQ ID | (SEQ | (SEQ ID | (SEQ |
| | NO: 776) | ID NO: | NO: 788) | ID NO: | NO: 800) | ID NO: | NO: 812) | ID NO: | NO: 824) | ID NO: |
| | | 782) | | 794) | | 806) | | 818) | | 830) |
| | GVVGV | FAVGV | GVRGV | FARGV | GVDGV | FADGV | GVAGV | FAAGV | GVCGV | FACGV |
| | GKSAL | GKSL | GKSAL | GKSL | GKSAL | GKSL | GKSAL | GKSL | GKSAL | GKSL |
| | (SEQ ID | (SEQ | (SEQ ID | (SEQ | (SEQ ID | (SEQ | (SEQ ID | (SEQ | (SEQ ID | (SEQ |
| | NO: 777) | ID NO: | NO: 789) | ID NO: | NO: 801) | ID NO: | NO: 813) | ID NO: | NO: 825) | ID NO: |
| | | 783) | | 795) | | 807) | | 819) | | 831) |

TABLE 14
Parental G12 sequences

| | |
|---|---|
| G12C | G12 Parental Long Sequence |
| | YKLVVVGACGVGKSALTIQLI (SEQ |
| | ID NO: 835) |
| G12V | YKLVVVGAVGVGKSALTIQLI |
| | (SEQ ID NO: 836) |
| G12A | YKLVVVGAAGVGKSALTIQLI (SEQ |
| | ID NO: 837) |
| G12R | YKLVVVGARGVGKSALTIQLI |
| | (SEQ ID NO: 838) |

### SUMMARY

It was demonstrated that altered peptide ligands (‘APLs’) in the pancreatic mouse model (Panc02) shows that APLs have distinct structural features measured by in silico modeling that enhances their immunogenicity in mice compared with the parental peptide (FIGS. 21A-21F). These data support the use of in silico structural modeling to engineer antigens.

It has been shown that Heteroclitic peptides, for example (10mer) for G12D which have been modeled for HLA 11*01, enhanced T cell response compared to the parental peptide (FIG. 22A-FIG. 22C and FIG. 23).

### OTHER EMBODIMENTS

From the foregoing description, it will be apparent that variations and modifications may be made to the disclosure described herein to adopt it to various usages and conditions. It is understood that certain method steps may be reordered, removed or modified to perform the methods disclosed herein. Such embodiments are also within the scope of the following claims.

All citations to sequences, patents and publications in this specification are herein incorporated by reference to the same extent as if each independent patent and publication was specifically and individually indicated to be incorporated by reference.

1-115. (canceled)

116. An immunogenic peptide or a nucleic acid sequence encoding the immunogenic peptide, wherein the immunogenic peptide comprises the amino acid sequence of any one of SEQ ID NOs: 2, 3, 182, 264, 270, 273, 280, 286, 532, 569, 577, 638, 649, 661, 774, 786, 798, 810, 822, 6-12, 14-21, 23-30, 32-39, 41-48, 50-57, 59-66, 68-75, 77-84, 86-93, 95-99, 101-105, 107-111, 113-117, 119-123, 125-129, 131-135, 137-141, 143-146, 148-152, 154-162, 165-168, 170-179, 183-185, 187-196, 198-202, 204-213, 215-219, 221-230, 232-236, 238-244, 246-252, 254-260, 262, 263, 265-268, 271, 272, 274-276, 278, 281-284, 287-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 533-536, 538-542, 544-548, 550-554, 556-560, 562-566, 570-573, 575-580, 582-587, 589-594, 596-601, 603-607, 609-613, 615-619, 621-625, 627-631, 633-637, 640-644, 646-648, 650, 651, 653-657, 659, 660, 662-664, 666-670, 672-677, 679-683, 685-690, 692-696, 698-703, 705-711, 713-718, 720-726, 728-733, 735-741, 743-748, 750-756, 758-763, 765-771, 773, 775-777, 779-783, 785, 787-789, 791-795, 797, 799-801, 803-807, 809, 811-813, 815-819, 821, 823-825, 827-831, 833, or 834.

117. The immunogenic peptide of claim 116, wherein the immunogenic peptide comprises the amino acid sequence of SEQ ID NOs: 2, 3, 182, 264, 270, 273, 280, 286, 532, 569, 577, 638, 649, 661, 774, 786, 798, 810, or 822.

118. The immunogenic peptide of claim 116, wherein the immunogenic peptide comprises the amino acid sequence of SEQ ID NOs: 2, 3, 182, 264, 280, 286, 577, 649, 774, or 798.

119. The immunogenic peptide of claim 116, wherein the immunogenic peptide comprises the amino acid sequence of SEQ ID NO: 2.

120. The immunogenic peptide of claim 116, wherein the immunogenic peptide comprises the amino acid sequence of SEQ ID NO: 3.

121. The immunogenic peptide of claim 116, wherein the immunogenic peptide comprises the amino acid sequence of SEQ ID NO: 264.

122. The immunogenic peptide of claim 116, wherein the nucleic acid sequence comprise ribonucleic acid.

123. A method of identifying heteroclitic neoepitopes having improved immunogenicity, the method comprising:
(a) modeling one or more immunogenic peptides onto a human leukocyte antigen (HLA) to form an HLA-peptide structure, wherein the modeling comprises identifying positional sequence similarity of peptide epitopes that bind to one or a plurality of HLA haplotypes;
(b) selecting the HLA-peptide structure having a sequence identity similar to a parental epitope being modeled;
(c) modeling heteroclitic neoepitopes into an HLA cleft by introducing amino acid rotamers with the lowest energy confirmation at each position;
(d) generating HLA-heteroclitic neoepitope complexes;
(e) validating the HLA-heteroclitic neoepitope complexes using a root-mean-square deviation analysis (Ca-RMSD); and
(f) performing a high-resolution Monte Carlo simulation with minimization docking of the heteroclitic neoepitope to the HLA cleft to identify the lowest full-atom energy conformation;
thereby identifying the heteroclitic neoepitopes having improved immunogenicity.

124. The method of claim 123, wherein the modeling one or more immunogenic peptides onto the HLA comprises aligning peptide sequences with a consensus binding motif of HLA molecules.

125. The method of claim 123, wherein the heteroclitic neoepitopes having improved immunogenicity comprises an amino acid sequence of any one or more of SEQ ID NOs: 1-838.

126. A method of treating cancer comprising administering to a subject in need thereof an immunogenic peptide or a nucleic acid encoding the immunogenic peptide, wherein the immunogenic peptide comprises the amino acid sequence of any one of SEQ ID NOs: 2, 3, 164, 181, 182, 270, 273, 264, 279, 280, 286, 532, 568, 569, 577, 638, 649, 661, 774, 786, 798, 810, 822, 5-12, 14-21, 23-30, 32-39, 41-48, 50-57, 59-66, 68-75, 77-84, 86-93, 95-99, 101-105, 107-111, 113-117, 119-123, 125-129, 131-135, 137-141, 143-146, 148-152, 154-162, 164-168, 170-179, 183-185, 187-196, 198-202, 204-213, 215-219, 221-230, 232-236, 238-244, 246-252, 254-260, 262, 263, 265268, 271, 272, 274-276, 278, 281-284, 287-292, 294-300, 302-308, 310-316, 318-324, 326-332, 334-340, 342-348, 350-356, 358-364, 366-372, 374-380, 382-388, 390-396, 398-404, 406-412, 414-420, 422-428, 430-436, 438-443, 445-450, 452-457, 459-464, 466-471, 473-478, 480-485, 487-492, 494-499, 501-506, 508-512, 514-518, 520-524, 526-530, 533-536, 538-542, 544-548, 550-554, 556-560, 562-566, 570-573, 575-580, 582-587, 589-594, 596-601, 603-607, 609-613, 615-619, 621-625, 627-631, 633-637, 640-644, 646-648, 650-651, 653-657, 659, 660, 662664, 666-670, 672-677, 679-683, 685-690, 692-696, 698-703, 705-711, 713-718, 720-726, 728-733, 735-741, 743-748, 750-756, 758-763, 765-771, 773, 775-777, 779-783, 785, 787-789, 791-795, 797, 799-801, 803-807, 809, 811-813, 815-819, 821, 823-825, 827-831, 833, or 834.

127. The method of claim 126, wherein the immunogenic peptide comprises the amino acid sequence of any one of SEQ ID NOs: 2, 3, 164, 181, 182, 264, 270, 273, 279, 280, 286, 532, 568, 569, 577, 649, 661, 774, 786, 798, 638, 810, or 822.

128. The method of claim 127, wherein the immunogenic peptide comprises the amino acid sequence of any one of SEQ ID NOs: 2, 3, 182, 264, 270, 273, 280, 286, 532, 569, 577, 649, 661, 774, 786, 798, 638, 810, or 822.

129. The method of claim 128, wherein the immunogenic peptide comprises the amino acid sequence of any one of SEQ ID NOs: 2, 3, 182, 264, 280, 286, 577, 649, 774, or 798.

130. The method of claim 129, wherein the immunogenic peptide comprises the amino acid sequence of SEQ ID NO: 2.

131. The method of claim 129, wherein the immunogenic peptide comprises the amino acid sequence of SEQ ID NO: 3.

132. The method of claim 126, wherein the nucleic acid sequence comprise ribonucleic acid (RNA).

133. The method of claim 132, wherein the RNA encodes two or more immunogenic peptide comprises the amino acid sequence of any one of SEQ ID NOs: 2, 3, 182, 264, 280, 286, 577, 649, 774, or 798.

134. The method of claim 132, wherein the RNA encodes an immunogenic peptide comprising the amino acid sequence of SEQ ID NOs: 2 or 3.

135. The method of claim 132, wherein the administering is via a vector.
